(19) United States
(12) Patent Application Publication
(10) Pub. No.: US 2024/0180972 A1
(43) Pub. Date: Jun. 6, 2024

(54) HUMAN PLURIPOTENT STEM CELL-DERIVED SECRETOME AS A BIOLOGIC FOR PREVENTION AND TREATMENT OF NEURODEGENERATIVE AND APOPTOTIC DISEASES

(71) Applicant: The Regents of the University of California, Oakland, CA (US)

(72) Inventors: Irina M. Conboy, El Sobrante, CA (US); Daehwan Kim, Albany, CA (US); Michael J. Conboy, El Sobrante, CA (US)

(73) Assignee: The Regents of the University of California, Oakland, CA (US)

(21) Appl. No.: 18/553,157

(22) PCT Filed: Apr. 1, 2022
(86) PCT No.: PCT/US2022/023109

§ 371 Date: Sep. 28, 2023

Related U.S. Application Data
(60) Provisional application No. 63/300,584, filed on Jan. 18, 2022.
(60) Provisional application No. 63/169,746, filed on Apr. 1, 2021.

(51) Int. Cl. A61K 35/545; A61K 9/00; A61K 31/727; A61K 38/17; A61P 25/28

(52) U.S. Cl. CPC A61K 35/545; A61K 9/0024; A61K 31/727; A61K 38/1709; A61P 25/28

(57) ABSTRACT

In various embodiments, neuroprotective compositions and uses thereof are provided. In certain embodiments, a method of protecting mammalian neurons from neurotoxins, oxidative stress or protein aggregation, mitochondrial dysfunction, inflammatory gene expression, induced cell death is provided where the method involves contacting the cells, such as neurons, with an effective amount of: a plurality of proteins that are secreted by induced pluripotent stem cells (iPSCs) derived from a healthy mammal and/or from a mammal with a disease, such as neurodegenerative pathology, exemplified by amyotrophic lateral sclerosis (ALS) where said proteins are secreted when said iPSC cells are pluripotent; and/or biologically active fragments of said proteins; and/or biologically active analogs of said proteins.

### CROSS-REFERENCE TO RELATED APPLICATIONS

This application claims priority to and benefit of U.S. Ser. No. 63/300,584, filed on Jan. 18, 2022, and to U.S. Ser. No. 63/169,746, filed on Apr. 1, 2021, both of which are incorporated herein by reference in their entirety for all purposes.

### STATEMENT OF GOVERNMENTAL SUPPORT

This invention was made with government support under AG058819, EB023776, and HL139605 awarded by the National Institutes of Health. The government has certain rights in the invention.

### INCORPORATION BY REFERENCE OF SEQUENCE LISTING PROVIDED AS A TEXT FILE

[Not Applicable]

### BACKGROUND

Neuronal cell death causes a plethora of neuro-degenerative diseases, including age-related loss of memory and dementias (such as Alzheimer's Disease), Parkinson's Disease, traumatic brain injury, and ALS. These diseases have in common an increase in reactive oxygen species (ROS), protein misfolding and aggregation, neuroinflammation, progressive-catastrophic loss of neurons, and so far, the lack of a cure.

ALS is a neurodegenerative disease characterized by a progressive loss of MNs, leading to paralysis, and respiratory failure. Most patients with ALS die within 2 to 5 years of diagnosis. More than 5000 patients in the United States receive a diagnosis of ALS each year, and an estimated 20,000 are living with the disease¹. The exact mechanisms of sporadic and familial ALS remain unknown, although mutations in superoxide dismutase (SOD1), oxidative damage and protein misfolding are involved^{2,3}. Anti-inflammatory and ROS reducing drugs (riluzole and edaravone⁴) slow down progression; later on, patients use respirators, speech facilitating devices, palliative care and ultimately, hospice care.

The first gene that was identified in association with ALS is superoxide dismutase 1 (SOD1) (3); mutations in SOD1: A5V, D90A, and G93A, retain catalytic activity and are implicated in neurotoxicity through SOD1 misfolding and altered redox catalysis (4-6). Interestingly, misfolded SOD1 proteins are characteristic of not only fALS but also in sALS, suggesting a strong link with the development of this pathology (7, 8). Moreover, oxidative stress induces SOD1 misfolding in wild-type cells and tissues (9-13). Thus, in a downward spiral, oxidative damage causes SOD1 misfolding, which exacerbates oxidative damage further through faulty redox catalysis and more oxidative damage.

A key feature of ALS is misfolding and mislocalization of proteins, such as TDP43, which is associated with increased oxidative damage and apoptosis (14-18). Perturbed proteostasis of TDP43 varies between sporadic and familial ALS (19-22), yet, several reports suggest a connection between SOD1 mutations and TDP-43 aggregation (23-27). Moreover, a recent report identified that mutant SOD1 physically binds to and induces the mislocalization of TDP-43, thereby causing degeneration of MNs in fALS (28).

Pluripotent stem cells (PSCs), e.g., embryonic stem cells (ESCs), and induced pluripotent stem cells (iPSCs), are used in various biomedical fields due to their capacity of unlimited self-renewal and the ability to differentiate into multiple cell types (29). However, control of stem cell differentiation remains limited, and concerns of oncogenic side-effects persist (30).

### SUMMARY

As described herein, we examined protective effects of conditioned media (CM) of hESC, hiPSC and ALS patients' derived hiPSCs on cells and animals that are exposed to oxidative damage. Our work demonstrates that secretome of hESCs, iPSCs and moreover, ALS patients' iPSCs, robustly protect neuronal cells from apoptosis, diminish mislocalization of TDP43, and significantly improve the formation and maintenance of neurites of ALS-MNs. Such neuro-protection manifests in the genetic and in an acquired neuro-toxicity models. Importantly, administration of CM form ALS-iPSCs (ALS iPSC-CM) to transgenic mice that model human disease (SOD1^{G93A}) prevented MN degeneration, maintained the innervation (neuro-muscular junctions), delayed onset of symptoms, and prolonged lifespan. Comparative proteomics and fractionation of conditioned medium outline specific proteins and fractions that are responsible for this neuroprotection. Translationally, this work suggests the rapid development of a new therapeutic for ALS and other neurodegenerative conditions.

Various embodiments provided herein may include, but need not be limited to, one or more of the following:

Embodiment 1: A method of protecting mammalian cells from oxidative stress, and/or mitochondrial dysfunction, and/or inflammatory gene expression, and/or protein aggregation, and/or toxin induced cell death, said method comprising:
contacting said cells with an effective amount of one or more of the following:
   a) unfractionated iPS cell derived secretome, where said iPS cells are derived from a mammal with a neurodegenerative pathology or a healthy mammal;
   b) heparin bound and/or unbound fractions of iPS cell derived secretome, where said iPS cells are derived from a mammal with a neurodegenerative pathology or a healthy mammal;
   c) a combination of soluble and exosome fractions of iPS cell derived secretome, where said iPS cells are derived from a mammal with a neurodegenerative pathology or a healthy mammal;
   d) proteins that are secreted by induced pluripotent stem cells (iPSCs) derived from a mammal with a neurodegenerative pathology or a healthy mammal where said proteins are secreted when said iPSC cells are pluripotent or biologically active fragments of said proteins and/or biologically active analogs of said proteins; and/or
   e) combinations of 2 or more proteins shown in Table 9, or biologically active fragments of said proteins and/or biologically active analogs of said proteins.

Embodiment 2: The method of embodiment 1, wherein said method comprises a method of protecting neuron from oxidative stress, and/or protein aggregation, and/or toxin-induced cell death.

Embodiment 3: The method of embodiment 1, wherein said method comprises contacting said cells with an effective amount of unfractionated iPS cell derived secretome, where said iPS cells are derived from a mammal with a neurodegenerative pathology and/or from a healthy mammal.

Embodiment 4: The method of embodiment 1, wherein said method comprises contacting said cells with an effective amount of heparin bound fraction of iPS cell derived secretome, where said iPS cells are derived from a mammal with a neurodegenerative pathology and/or a from a healthy mammal.

Embodiment 5: The method of embodiment 1, wherein said method comprises contacting said neurons with an effective amount of heparin unbound fraction of iPS cell derived secretome, where said iPS cells are derived from a mammal with a neurodegenerative pathology and/or from a healthy mammal.

Embodiment 6: The method of embodiment 1, wherein said method comprises contacting said neurons with an effective amount of a plurality of proteins that are secreted by induced pluripotent stem cells (iPSCs) derived from a mammal with a neurodegenerative pathology and/or from a healthy mammal where said proteins are secreted when said iPSC cells are pluripotent or biologically active fragments of said proteins.

Embodiment 7: The method according to any one of embodiments 1-6, wherein said cells comprise neurons.

Embodiment 8: The method according to any one of embodiments 1-7, wherein said iPSCs are derived from a mammal with a neurodegenerative pathology.

Embodiment 9: The method according to any one of embodiments 1-7, wherein said iPSCs are derived from a healthy mammal.

Embodiment 10: The method according to any one of embodiments 1-9, wherein:
said iPS cell derived secretome; and/or
said heparin bound fraction of iPS cell derived secretome; and/or
said heparin unbound fraction of iPS cell derived secretome; and/or
said proteins secreted by induced pluripotent stem cells (iPS cells); and/or
are from iPS cells derived from a healthy mammal and/or from a mammal having a neuropathology selected from the group consisting of a motor neuron disease, Alzheimer's disease, Parkinson's disease, Huntington's disease, HIV-1 associated neurological degeneration, neurodegeneration associated with an ischemic event, neurodegeneration associated with traumatic brain injury (TBI), neurodegeneration associated with a spinal cord injury, drug-environmental toxin-induced neurodegeneration, and glaucoma.

Embodiment 11: The method of embodiment 10, wherein said neuropathology comprises a pathology selected from the group consisting of a motor neuron disease, Alzheimer's disease, Parkinson's disease, Huntington's disease, HIV-1 associated neurological degeneration, neurodegeneration associated with an ischemic event, neurodegeneration associated with traumatic brain injury (TBI), neurodegeneration associated with a spinal cord injury, drug-environmental toxin-induced neurodegeneration, and glaucoma.

Embodiment 12: The method of embodiment 11, wherein said neuropathology comprises a motor neuron disease selected from the group consisting of Amyotrophic lateral sclerosis (ALS), spinal muscular atrophy (SMA), progressive bulbar palsy (PBP), primary lateral sclerosis (PLS), pseudobulbar palsy, and progressive muscular atrophy (PMA).

Embodiment 13: The method of embodiment 12, wherein said motor neuron disease comprises ALS.

Embodiment 14: The method of embodiment 12, wherein said motor neuron disease comprises SMA.

Embodiment 15: The method according to any one of embodiments 1-14, wherein said proteins comprise or consist of proteins whose production and/or secretion by IPS cells is reduced or absent when said iPSCs differentiate into the embryoid bodies or their derived lineages, such as fibroblasts.

Embodiment 16: The method according to any one of embodiments 1-15, wherein said proteins comprise or consist of proteins whose neuroprotective activities are reduced or absent when said iPSCs differentiate into the embryoid bodies or their derived lineages, such as fibroblasts.

Embodiment 17: The method according to any one of embodiments 1-16, wherein said plurality of proteins comprises a protein selected from the group consisting of angiogenin (ANG), Angiopoietin-1(ANG-1), angiopoietin like 3, apolipoprotein E (ApoE), secreted frizzled related protein 1, serpin family A member 5, thrombospondin 4, vascular endothelial growth factor B, fibroblast growth factor 11 (FGF11), and fibroblast growth factor 19 (FGF19), one or more proteins of Table 9, or biologically active fragments or analogs thereof.

Embodiment 18: The method of embodiment 17, wherein said plurality of proteins comprises angiogenin (ANG), or a biologically active fragment or analog thereof.

Embodiment 19: The method according to any one of embodiments 17-18, wherein said plurality of proteins comprises Angiopoietin-1(ANG-1), or a biologically active fragment or analog thereof.

Embodiment 20: The method according to any one of embodiments 17-19, wherein said plurality of proteins comprises angiopoietin like 3, or a biologically active fragment or analog thereof.

Embodiment 21: The method according to any one of embodiments 17-20, wherein said plurality of proteins comprises apolipoprotein E (ApoE), or a biologically active fragment or analog thereof.

Embodiment 22: The method according to any one of embodiments 17-21, wherein said plurality of proteins comprises secreted frizzled related protein 1, or a biologically active fragment or analog thereof.

Embodiment 23: The method according to any one of embodiments 17-22, wherein said plurality of proteins comprises serpin family A member 5, or a biologically active fragment or analog thereof.

Embodiment 24: The method according to any one of embodiments 17-23, wherein said plurality of proteins comprises thrombospondin 4, or a biologically active fragment or analog thereof.

Embodiment 25: The method according to any one of embodiments 17-24, wherein said plurality of proteins comprises vascular endothelial growth factor B, or a biologically active fragment or analog thereof.

Embodiment 26: The method according to any one of embodiments 17-25, wherein said plurality of proteins comprises fibroblast growth factor 11 (FGF11), or a biologically active fragment or analog thereof.

Embodiment 27: The method according to any one of embodiments 17-26, wherein said plurality of proteins comprises fibroblast growth factor 19 (FGF19), or a biologically active fragment or analog thereof.

Embodiment 28: The method according to any one of embodiments 1-27, wherein said plurality of proteins comprises a protein selected from the group consisting of ANG-1, ANG, IGFBP-2, TIMP-1, SERPINA5, FGF19, and ApoE, or biologically active fragments or analogs thereof.

Embodiment 29: The method of embodiment 28, wherein said plurality of proteins comprises ANG-1, or a biologically active fragment or analog thereof.

Embodiment 30: The method according to any one of embodiments 28-29, wherein said plurality of proteins comprises ANG, or a biologically active fragment or analog thereof.

Embodiment 31: The method according to any one of embodiments 28-30, wherein said plurality of proteins comprises IGFBP-2, or a biologically active fragment or analog thereof.

Embodiment 32: The method according to any one of embodiments 28-31, wherein said plurality of proteins comprises TIMP-1, or a biologically active fragment or analog thereof.

Embodiment 33: The method according to any one of embodiments 28-32, wherein said plurality of proteins comprises SERPINA5, or a biologically active fragment or analog thereof.

Embodiment 34: The method according to any one of embodiments 28-33, wherein said plurality of proteins comprises FGF19, or a biologically active fragment or analog thereof.

Embodiment 35: The method according to any one of embodiments 28-34, wherein said plurality of proteins comprises ApoE, or a biologically active fragment or analog thereof.

Embodiment 36: The method according to any one of embodiments 1-27, wherein said plurality of proteins comprises two or more proteins shown in Table 9 or biologically active fragments or analogs thereof.

Embodiment 37: The method of embodiment 36, wherein said two or more proteins comprise Aldolase C or a biologically active fragment or analog thereof.

Embodiment 38: The method according to any one of embodiments 36-37, wherein said two or more proteins comprise Alpha Lactalbumin or a biologically active fragment or analog thereof.

Embodiment 39: The method according to any one of embodiments 36-38, wherein said two or more proteins comprise ANGPTL3 or a biologically active fragment or analog thereof.

Embodiment 40: The method according to any one of embodiments 36-39, wherein said two or more proteins comprise ApoA2 or a biologically active fragment or analog thereof.

Embodiment 41: The method according to any one of embodiments 36-40, wherein said two or more proteins comprise ApoE or a biologically active fragment or analog thereof.

Embodiment 42: The method according to any one of embodiments 36-41, wherein said two or more proteins comprise BCAM or a biologically active fragment or analog thereof.

Embodiment 43: The method according to any one of embodiments 36-42, wherein said two or more proteins comprise MUC1 or a biologically active fragment or analog thereof.

Embodiment 44: The method according to any one of embodiments 36-43, wherein said two or more proteins comprise MUC16 or a biologically active fragment or analog thereof.

Embodiment 45: The method according to any one of embodiments 36-44, wherein said two or more proteins comprise Caspase-3 or a biologically active fragment or analog thereof.

Embodiment 46: The method according to any one of embodiments 36-45, wherein said two or more proteins comprise Cathepsin B or a biologically active fragment or analog thereof.

Embodiment 47: The method according to any one of embodiments 36-46, wherein said two or more proteins comprise CBP or a biologically active fragment or analog thereof.

Embodiment 48: The method according to any one of embodiments 36-47, wherein said two or more proteins comprise TfR or a biologically active fragment or analog thereof.

Embodiment 49: The method according to any one of embodiments 36-48, wherein said two or more proteins comprise CEA or a biologically active fragment or analog thereof.

Embodiment 50: The method according to any one of embodiments 36-49, wherein said two or more proteins comprise Ceruloplasmin or a biologically active fragment or analog thereof.

Embodiment 51: The method according to any one of embodiments 36-50, wherein said two or more proteins comprise Chemerin or a biologically active fragment or analog thereof.

Embodiment 52: The method according to any one of embodiments 36-51, wherein said two or more proteins comprise CHI3L1 or a biologically active fragment or analog thereof.

Embodiment 53: The method according to any one of embodiments 36-52, wherein said two or more proteins comprise CK-MB or a biologically active fragment or analog thereof.

Embodiment 54: The method according to any one of embodiments 36-53, wherein said two or more proteins comprise Clusterin or a biologically active fragment or analog thereof.

Embodiment 55: The method according to any one of embodiments 36-54, wherein said two or more proteins comprise C2 or a biologically active fragment or analog thereof.

Embodiment 56: The method according to any one of embodiments 36-55, wherein said two or more proteins comprise C5a or a biologically active fragment or analog thereof.

Embodiment 57: The method according to any one of embodiments 36-56, wherein said two or more proteins comprise Corticosteroid-binding globulin or a biologically active fragment or analog thereof.

Embodiment 58: The method according to any one of embodiments 36-57, wherein said two or more proteins comprise C-Peptide or a biologically active fragment or analog thereof.

Embodiment 59: The method according to any one of embodiments 36-58, wherein said two or more proteins comprise Troponin T or a biologically active fragment or analog thereof.

Embodiment 60: The method according to any one of embodiments 36-59, wherein said two or more proteins comprise Cytokeratin 19 or a biologically active fragment or analog thereof.

Embodiment 61: The method according to any one of embodiments 36-60, wherein said two or more proteins comprise BNP or a biologically active fragment or analog thereof.

Embodiment 62: The method according to any one of embodiments 36-61, wherein said two or more proteins comprise ACTH or a biologically active fragment or analog thereof.

Embodiment 63: The method according to any one of embodiments 36-62, wherein said two or more proteins comprise Exostosin-like 2 or a biologically active fragment or analog thereof.

Embodiment 64: The method according to any one of embodiments 36-63, wherein said two or more proteins comprise Ferritin or a biologically active fragment or analog thereof.

Embodiment 65: The method according to any one of embodiments 36-64, wherein said two or more proteins comprise Fibrinopeptide A or a biologically active fragment or analog thereof.

Embodiment 66: The method according to any one of embodiments 36-65, wherein said two or more proteins comprise FSH or a biologically active fragment or analog thereof.

Embodiment 67: The method according to any one of embodiments 36-66, wherein said two or more proteins comprise GLP-1 or a biologically active fragment or analog thereof.

Embodiment 68: The method according to any one of embodiments 36-67, wherein said two or more proteins comprise GMNN or a biologically active fragment or analog thereof.

Embodiment 69: The method according to any one of embodiments 36-68, wherein said two or more proteins comprise Hemopexin or a biologically active fragment or analog thereof.

Embodiment 70: The method according to any one of embodiments 36-69, wherein said two or more proteins comprise HSP27 or a biologically active fragment or analog thereof.

Embodiment 71: The method according to any one of embodiments 36-70, wherein said two or more proteins comprise HSP90 or a biologically active fragment or analog thereof.

Embodiment 72: The method according to any one of embodiments 36-71, wherein said two or more proteins comprise IL-34 or a biologically active fragment or analog thereof.

Embodiment 73: The method according to any one of embodiments 36-72, wherein said two or more proteins comprise Kallikrein 2 or a biologically active fragment or analog thereof.

Embodiment 74: The method according to any one of embodiments 36-73, wherein said two or more proteins comprise Kallikrein 10 or a biologically active fragment or analog thereof.

Embodiment 75: The method according to any one of embodiments 36-74, wherein said two or more proteins comprise Lyn or a biologically active fragment or analog thereof.

Embodiment 76: The method according to any one of embodiments 36-75, wherein said two or more proteins comprise NPTXR or a biologically active fragment or analog thereof.

Embodiment 77: The method according to any one of embodiments 36-76, wherein said two or more proteins comprise P-Cadherin or a biologically active fragment or analog thereof.

Embodiment 78: The method according to any one of embodiments 36-77, wherein said two or more proteins comprise PIM2 or a biologically active fragment or analog thereof.

Embodiment 79: The method according to any one of embodiments 36-78, wherein said two or more proteins comprise PPARg2 or a biologically active fragment or analog thereof.

Embodiment 80: The method according to any one of embodiments 36-79, wherein said two or more proteins comprise PR Isoform B or a biologically active fragment or analog thereof.

Embodiment 81: The method according to any one of embodiments 36-80, wherein said two or more proteins comprise PSA-free or a biologically active fragment or analog thereof.

Embodiment 82: The method according to any one of embodiments 36-81, wherein said two or more proteins comprise PTPRD or a biologically active fragment or analog thereof.

Embodiment 83: The method according to any one of embodiments 36-82, wherein said two or more proteins comprise Ret or a biologically active fragment or analog thereof.

Embodiment 84: The method according to any one of embodiments 36-83, wherein said two or more proteins comprise Serpin A5 or a biologically active fragment or analog thereof.

Embodiment 85: The method according to any one of embodiments 36-84, wherein said two or more proteins comprise SHBG or a biologically active fragment or analog thereof.

Embodiment 86: The method according to any one of embodiments 36-85, wherein said two or more proteins comprise SOX2 or a biologically active fragment or analog thereof.

Embodiment 87: The method according to any one of embodiments 36-86, wherein said two or more proteins comprise Angiogenin or a biologically active fragment or analog thereof.

Embodiment 88: The method according to any one of embodiments 36-87, wherein said two or more proteins comprise Angiopoietin-1 or a biologically active fragment or analog thereof.

Embodiment 89: The method according to any one of embodiments 36-88, wherein said two or more proteins comprise CCR5 or a biologically active fragment or analog thereof.

Embodiment 90: The method according to any one of embodiments 36-89, wherein said two or more proteins comprise CD40 Ligand or a biologically active fragment or analog thereof.

Embodiment 91: The method according to any one of embodiments 36-90, wherein said two or more proteins comprise Chordin-Like-1 or a biologically active fragment or analog thereof.

Embodiment 92: The method according to any one of embodiments 36-91, wherein said two or more proteins comprise Cripto-1 or a biologically active fragment or analog thereof.

Embodiment 93: The method according to any one of embodiments 36-92, wherein said two or more proteins comprise CXCR6 or a biologically active fragment or analog thereof.

Embodiment 94: The method according to any one of embodiments 36-93, wherein said two or more proteins comprise EG-VEGF or a biologically active fragment or analog thereof.

Embodiment 95: The method according to any one of embodiments 36-94, wherein said two or more proteins comprise ErbB3 or a biologically active fragment or analog thereof.

Embodiment 96: The method according to any one of embodiments 36-95, wherein said two or more proteins comprise FGF-11 or a biologically active fragment or analog thereof.

Embodiment 97: The method according to any one of embodiments 36-96, wherein said two or more proteins comprise FGF-19 or a biologically active fragment or analog thereof.

Embodiment 98: The method according to any one of embodiments 36-97, wherein said two or more proteins comprise GDF-9 or a biologically active fragment or analog thereof.

Embodiment 99: The method according to any one of embodiments 36-98, wherein said two or more proteins comprise GDF-11 or a biologically active fragment or analog thereof.

Embodiment 100: The method according to any one of embodiments 36-99, wherein said two or more proteins comprise Granzyme A or a biologically active fragment or analog thereof.

Embodiment 101: The method according to any one of embodiments 36-100, wherein said two or more proteins comprise GRO or a biologically active fragment or analog thereof.

Embodiment 102: The method according to any one of embodiments 36-101, wherein said two or more proteins comprise HCR or a biologically active fragment or analog thereof.

Embodiment 103: The method according to any one of embodiments 36-102, wherein said two or more proteins comprise NRG1 Isoform GGF2 or a biologically active fragment or analog thereof.

Embodiment 104: The method according to any one of embodiments 36-103, wherein said two or more proteins comprise IFN-alpha/beta R2 or a biologically active fragment or analog thereof.

Embodiment 105: The method according to any one of embodiments 36-104, wherein said two or more proteins comprise IGFBP-2 or a biologically active fragment or analog thereof.

Embodiment 106: The method according to any one of embodiments 36-105, wherein said two or more proteins comprise IL-38 or a biologically active fragment or analog thereof.

Embodiment 107: The method according to any one of embodiments 36-106, wherein said two or more proteins comprise IL-7 or a biologically active fragment or analog thereof.

Embodiment 108: The method according to any one of embodiments 36-107, wherein said two or more proteins comprise IL-17 RB or a biologically active fragment or analog thereof.

Embodiment 109: The method according to any one of embodiments 36-108, wherein said two or more proteins comprise IL-17 RD or a biologically active fragment or analog thereof.

Embodiment 110: The method according to any one of embodiments 36-109, wherein said two or more proteins comprise IL-21 or a biologically active fragment or analog thereof.

Embodiment 111: The method according to any one of embodiments 36-110, wherein said two or more proteins comprise IL-23 R or a biologically active fragment or analog thereof.

Embodiment 112: The method according to any one of embodiments 36-111, wherein said two or more proteins comprise IL-26 or a biologically active fragment or analog thereof.

Embodiment 113: The method according to any one of embodiments 36-112, wherein said two or more proteins comprise IL-29 or a biologically active fragment or analog thereof.

Embodiment 114: The method according to any one of embodiments 36-113, wherein said two or more proteins comprise Insulysin or a biologically active fragment or analog thereof.

Embodiment 115: The method according to any one of embodiments 36-114, wherein said two or more proteins comprise Kremen-2 or a biologically active fragment or analog thereof.

Embodiment 116: The method according to any one of embodiments 36-115, wherein said two or more proteins comprise MFRP or a biologically active fragment or analog thereof.

Embodiment 117: The method according to any one of embodiments 36-116, wherein said two or more proteins comprise MIF or a biologically active fragment or analog thereof.

Embodiment 118: The method according to any one of embodiments 36-117, wherein said two or more proteins comprise MIP 2 or a biologically active fragment or analog thereof.

Embodiment 119: The method according to any one of embodiments 36-118, wherein said two or more proteins comprise MMP-9 or a biologically active fragment or analog thereof.

Embodiment 120: The method according to any one of embodiments 36-119, wherein said two or more proteins comprise MMP-16 or a biologically active fragment or analog thereof.

Embodiment 121: The method according to any one of embodiments 36-120, wherein said two or more proteins comprise MMP-25 or a biologically active fragment or analog thereof.

Embodiment 122: The method according to any one of embodiments 36-121, wherein said two or more proteins comprise NRG1 Isoform GGF2 or a biologically active fragment or analog thereof.

Embodiment 123: The method according to any one of embodiments 36-122, wherein said two or more proteins comprise Orexin-A or a biologically active fragment or analog thereof.

Embodiment 124: The method according to any one of embodiments 36-123, wherein said two or more proteins comprise Oncostatin M or a biologically active fragment or analog thereof.

Embodiment 125: The method according to any one of embodiments 36-124, wherein said two or more proteins comprise OX40 Ligand or a biologically active fragment or analog thereof.

Embodiment 126: The method according to any one of embodiments 36-125, wherein said two or more proteins comprise PDGF-AA or a biologically active fragment or analog thereof.

Embodiment 127: The method according to any one of embodiments 36-126, wherein said two or more proteins comprise PDGF-C or a biologically active fragment or analog thereof.

Embodiment 128: The method according to any one of embodiments 36-127, wherein said two or more proteins comprise ROBO4 or a biologically active fragment or analog thereof.

Embodiment 129: The method according to any one of embodiments 36-128, wherein said two or more proteins comprise sFRP-1 or a biologically active fragment or analog thereof.

Embodiment 130: The method according to any one of embodiments 36-129, wherein said two or more proteins comprise SIGIRR or a biologically active fragment or analog thereof.

Embodiment 131: The method according to any one of embodiments 36-130, wherein said two or more proteins comprise SMAD1 or a biologically active fragment or analog thereof.

Embodiment 132: The method according to any one of embodiments 36-131, wherein said two or more proteins comprise SMAD4 or a biologically active fragment or analog thereof.

Embodiment 133: The method according to any one of embodiments 36-132, wherein said two or more proteins comprise SMAD5 or a biologically active fragment or analog thereof.

Embodiment 134: The method according to any one of embodiments 36-133, wherein said two or more proteins comprise SMAD7 or a biologically active fragment or analog thereof.

Embodiment 135: The method according to any one of embodiments 36-134, wherein said two or more proteins comprise Spinesin or a biologically active fragment or analog thereof.

Embodiment 136: The method according to any one of embodiments 36-135, wherein said two or more proteins comprise TACI or a biologically active fragment or analog thereof.

Embodiment 137: The method according to any one of embodiments 36-136, wherein said two or more proteins comprise Thrombospondin-4 or a biologically active fragment or analog thereof.

Embodiment 138: The method according to any one of embodiments 36-137, wherein said two or more proteins comprise TIMP-1 or a biologically active fragment or analog thereof.

Embodiment 139: The method according to any one of embodiments 36-138, wherein said two or more proteins comprise TLR1 or a biologically active fragment or analog thereof.

Embodiment 140: The method according to any one of embodiments 36-139, wherein said two or more proteins comprise TRADDor a biologically active fragment or analog thereof.

Embodiment 141: The method according to any one of embodiments 36-140, wherein said two or more proteins comprise TWEAK or a biologically active fragment or analog thereof.

Embodiment 142: The method according to any one of embodiments 36-141, wherein said two or more proteins comprise VEGF-B or a biologically active fragment or analog thereof.

Embodiment 143: The method according to any one of embodiments 17-142, wherein said protein(s) comprise full-length proteins.

Embodiment 144: The method according to any one of embodiments 17-142, wherein said protein(s) comprise biologically active fragments of said proteins.

Embodiment 145: The method according to any one of embodiments 17-142, wherein said protein(s) comprise analogs of said proteins.

Embodiment 146: The method according to any one of embodiments 1-145, wherein said composition is effective to reduce cell apoptosis as compared to an untreated control.

Embodiment 147: The method of embodiment 146, wherein said composition is effective to reduce neuronal apoptosis as compared to an untreated control.

Embodiment 148: The method according to any one of embodiments 1-145, wherein said composition is effective to diminish aggregation of TDP43 as compared to an untreated control.

Embodiment 149: The method according to any one of embodiments 1-145, wherein said composition is effective to diminish neurite shrinkage as compared to an untreated control.

Embodiment 150: The method according to any one of embodiments 1-145, wherein said composition is effective to diminish mitochondrial dysfunction as compared to an untreated control.

Embodiment 151: The method according to any one of embodiments 1-145, wherein said composition is effective to diminish inflammatory gene expression as compared to an untreated control.

Embodiment 152: The method according to any one of embodiments 1-151, wherein said contacting comprises administering said plurality of proteins and/or biologically active fragments of said proteins and/or analogs thereof to a mammal in need thereof.

Embodiment 153: The method of embodiment 152, wherein said mammal in need thereof comprises a mammal diagnosed as having or as at risk for a neurodegenerative pathology or mitochondrial disease.

Embodiment 154: The method of embodiment 153, wherein said mammal in need thereof comprises a mammal having or at risk for a neurodegenerative pathology selected from the group consisting of a motor neuron disease, Alzheimer's disease, Parkinson's disease, Huntington's disease, HIV-1 associated neurological degeneration, neurodegeneration associated with an ischemic event, neurodegeneration associated with traumatic brain injury (TBI), neurodegeneration associated with a spinal cord injury, drug-environmental toxin-induced neurodegeneration, and glaucoma.

Embodiment 155: The method of embodiment 154, wherein said neurodegenerative pathology comprises a motor neuron disease.

Embodiment 156: The method of embodiment 155, wherein said motor neuron disease comprises a motor neuron disease selected from the group consisting of wherein said motor neuron disease comprises a disease selected from the group consisting of Amyotrophic lateral sclerosis (ALS), spinal muscular atrophy (SMA type I, and/or type II, and/or type III, and/or congenital SMA), progressive bulbar palsy (PBP), pseudobulbar palsy, primary lateral sclerosis (PLS), progressive muscular atrophy (PMA), and Fazio-Londe disease.

Embodiment 157: The method of embodiment 155, wherein said motor neuron disease comprises amyotrophic lateral sclerosis (ALS).

Embodiment 158: The method of embodiment 155, wherein said motor neuron disease comprises SMA.

Embodiment 159: The method of embodiment 154, wherein said neurodegenerative pathology comprises methamphetamine-associated neurodegeneration.

Embodiment 160: The method of embodiment 154, wherein said neurodegenerative pathology comprises toxin-induced (e.g., environmental toxin-induced) neurodegeneration.

Embodiment 161: The method of embodiment 154, wherein said neurodegenerative pathology comprises Alzheimer's disease.

Embodiment 162: The method of embodiment 154, wherein said neurodegenerative pathology comprises Parkinson's disease.

Embodiment 163: The method of embodiment 154, wherein said neurodegenerative pathology comprises neurodegeneration associated with a stroke.

Embodiment 164: The method of embodiment 154, wherein said neurodegenerative pathology comprises neurodegeneration associated with a spinal cord injury.

Embodiment 165: The method of embodiment 154, wherein said neurodegenerative pathology comprises neurodegeneration associated with a traumatic brain injury.

Embodiment 166: The method of embodiment 154, wherein said neurodegenerative pathology comprises glaucoma.

Embodiment 167: The method of embodiment 153, wherein said mammal in need thereof comprises a mammal having or at risk for a mitochondrial disease.

Embodiment 168: The method of embodiment 167, wherein said mitochondrial disease comprises a disease selected from the group consisting of Autosomal dominant optic atrophy, Alpers Disease or Syndrome, Barth Syndrome, Beta-oxidation defects, Carnitine Deficiency, Carnitine-Acyl-Carnitine Deficiency, Chronic Progressive External Ophthalmoplegia Syndrome (CPEO), Co-Enzyme Q10 Deficiency, Complex I Deficiency, Complex II Deficiency, Complex III Deficiency/COX Deficiency, Complex V Deficiency, CPT I Deficiency, CPT II Deficiency, Creatine Deficiency Syndrome, Kearns-Sayre Syndrome (KSS), Lactic Acidosis, Leukodystrophy (LBSL), LCHA Deficiency, Leber Hereditary Optic Neuropathy, Leigh Disease or syndrome, Long-Chain Acyl-CoA Dehydrongenase Deficiency (LCA Deficiency), Luft Disease, MAD/Glutaric Aciduria Type II, Medium-Chain Acyl-CoA Dehydrongenase Deficiency (MCAD), Mitochondrial Encephalomyopathy Lactic Acidosis and Stroke-like Episodes (MELAS), Mitochondrial DNA Depletion, Mitochondrial Encephalopathy, Mitochondrial Enoyl CoA Reductase Protein Associated Neurodegeneration (MEPAN), Mitochondrial Recessive Ataxia Syndrome (MIRAS), Myoclonic Epilepsy and Ragged-Red Fiber Disease (MERRF), Myoneurogastointestinal Disorder and Encephalopathy (MNGIE), Neuropathy, Ataxia, and Retinitis Pigmentosa (NARP), Neuropathy, Ataxia, and Retinitis Pigmentosa (NARP), Pearson Syndrome, Pyruvate Carboxylase Deficiency, Pyruvate Carboxylase Deficiency, Pyruvate Dehydrogenase Deficiency (PDC deficiency), Pyruvate Dehydrogenase Deviciency (PDC Deficiency), SANDO, SCHAD, Short-Chain Acyl-CoA Dehydrogenase Deficiency (SCAD), TK2/myopathic form, and Very Long-Chain Acyl-CoA Dehydrogenase Deficiency (VLCAD).

Embodiment 169: The method according to any one of embodiments 152-168, wherein said mammal is a human.

Embodiment 170: The method according to any one of embodiments 152-168, wherein said mammal is a non-human mammal.

Embodiment 171: The method according to any one of embodiments 152-170, wherein said neuroprotection comprises protection of cortical neurons.

Embodiment 172: The method according to any one of embodiments 152-171, wherein said neuroprotection comprises protection of hippocampal neurons.

Embodiment 173: The method according to any one of embodiments 152-172, wherein said neuroprotection comprises protection of dopaminergic neurons.

Embodiment 174: The method according to any one of embodiments 152-173, wherein said neuroprotection comprises protection of spinal cord neurons.

Embodiment 175: The method according to any one of embodiments 152-174, wherein said neuroprotection comprises protection of motor neurons.

Embodiment 176: The method according to any one of embodiments 152-175, wherein said contacting comprises administration to a mammal via a route selected from the group consisting of oral delivery, isophoretic delivery, transdermal delivery, parenteral delivery, aerosol administration, administration via inhalation, intravenous administration, and rectal administration.

Embodiment 177: The method according to any one of embodiments 152-175, wherein said contacting comprises administration to the brain or spinal cord of a mammal.

Embodiment 178: The method according to any one of embodiments 152-175, wherein said contacting comprises intracerebral, ventricular or intrathecal delivery to a mammal.

Embodiment 179: The method according to any one of embodiments 152-175, wherein said contacting comprises intranasal delivery to a mammal.

Embodiment 180: The method according to any one of embodiments 152-175, wherein said contacting comprises delivery via an implant in a mammal.

Embodiment 181: The method according to any one of embodiments 1-180, wherein said plurality secretome, secretome fractions, proteins and/or biologically active fragments thereof, and/or analogs thereof are effective to inhibit apoptosis, and/or to attenuate oxidative stress, and/or to repair oxidative damage, and/or prevent protein aggregation and mislocalization and/or prevent loss of axonal, etc. neuron projections, and/or prevent denervation, and/or increase innervation, muscle mass, neuro-muscular function, prevent and attenuate paralysis and morbidity, when administered to a mammal.

Embodiment 182: A neuroprotective composition comprising:
a plurality of proteins (unfractionated or fractionated) that are secreted by induced pluripotent stem cells (iPSCs) derived from a mammal with a neuropathology and/or mitochondrial disease and/or from a healthy mammal, where said proteins are secreted when said iPSC cells are pluripotent; and/or
biologically active fragments of said secreted proteins; and/or
biologically active analogs of said secreted proteins.

Embodiment 183: The neuroprotective composition of embodiment 182, wherein said mitochondrial disease comprises a disease selected from the group consisting of Autosomal dominant optic atrophy, Alpers Disease or Syndrome, Barth Syndrome, Beta-oxidation defects, Carnitine Deficiency, Carnitine-Acyl-Carnitine Deficiency, Chronic Progressive External Ophthalmoplegia Syndrome (CPEO), Co-Enzyme Q10 Deficiency, Complex I Deficiency, Complex II Deficiency, Complex III Deficiency/COX Deficiency, Complex V Deficiency, CPT I Deficiency, CPT II Deficiency, Creatine Deficiency Syndrome, Kearns-Sayre Syndrome (KSS), Lactic Acidosis, Leukodystrophy (LBSL), LCHA Deficiency, Leber Hereditary Optic Neuropathy, Leigh Disease or syndrome, Long-Chain Acyl-CoA Dehydrongenase Deficiency (LCA Deficiency), Luft Disease, MAD/Glutaric Aciduria Type II, Medium-Chain Acyl-CoA Dehydrongenase Deficiency (MCAD), Mitochondrial Encephalomyopathy Lactic Acidosis and Stroke-like Episodes (MELAS), Mitochondrial DNA Depletion, Mitochondrial Encephalopathy, Mitochondrial Enoyl CoA Reductase Protein Associated Neurodegeneration (MEPAN), Mitochondrial Recessive Ataxia Syndrome (MIRAS), Myoclonic Epilepsy and Ragged-Red Fiber Disease (MERRF), Myoneurogastointestinal Disorder and Encephalopathy (MNGIE), Neuropathy, Ataxia, and Retinitis Pigmentosa (NARP), Neuropathy, Ataxia, and Retinitis Pigmentosa (NARP), Pearson Syndrome, Pyruvate Carboxylase Deficiency, Pyruvate Carboxylase Deficiency, Pyruvate Dehydrogenase Deficiency (PDC deficiency), Pyruvate Dehydrogenase Deviciency (PDC Deficiency), SANDO, SCHAD, Short-Chain Acyl-CoA Dehydrogenase Deficiency (SCAD), TK2/myopathic form, and Very Long-Chain Acyl-CoA Dehydrogenase Deficiency (VLCAD).

Embodiment 184: The neuroprotective composition of embodiment 182, wherein said neuropathology is a neuropathology selected from the group consisting of a motor neuron disease, Alzheimer's disease, Parkinson's disease, Huntington's disease, HIV-1 associated neurological degeneration, neurodegeneration associated with an ischemic event, neurodegeneration associated with traumatic brain injury (TBI), neurodegeneration associated with a spinal cord injury, drug-environmental toxin-induced neurodegeneration, and glaucoma.

Embodiment 185: The neuroprotective composition of embodiment 184, wherein said neuropathology comprises a motor neuron disease.

Embodiment 186: The neuroprotective composition of embodiment 185, wherein said motor neuron disease comprises a disease selected from the group consisting of Amyotrophic lateral sclerosis (ALS), spinal muscular atrophy (SMA), progressive bulbar palsy (PBP), primary lateral sclerosis (PLS), pseudobulbar palsy, and progressive muscular atrophy (PMA).

Embodiment 187: The neuroprotective composition of embodiment 185, wherein said motor neuron disease comprises ALS.

Embodiment 188: The neuroprotective composition of embodiment 185, wherein said motor neuron disease comprises SMA.

Embodiment 189: The neuroprotective composition according to any one of embodiments 182-188, wherein said plurality of proteins comprise or consist of proteins whose activity and/or levels are reduced or absent when said iPSCs differentiate along the embryonic fibroblast lineage.

Embodiment 190: The neuroprotective composition according to any one of embodiments 182-189, wherein said plurality of proteins comprises a protein selected from the group consisting of angiogenin (ANG), Angiopoietin-1(ANG-1), angiopoietin like 3, apolipoprotein E (ApoE), secreted frizzled related protein 1, serpin family A member 5, thrombospondin 4, vascular endothelial growth factor B, fibroblast growth factor 11 (FGF11), and fibroblast growth factor 19 (FGF19), or biologically active fragments or analogs thereof.

Embodiment 191: The neuroprotective composition of embodiment 190, wherein said plurality of proteins comprises angiogenin (ANG), or a biologically active fragment or analog thereof.

Embodiment 192: The neuroprotective composition according to any one of embodiments 190-191, wherein said plurality of proteins comprises Angiopoietin-1(ANG-1), or a biologically active fragment or analog thereof.

Embodiment 193: The neuroprotective composition according to any one of embodiments 190-192, wherein said plurality of proteins comprises angiopoietin like 3, or a biologically active fragment or analog thereof.

Embodiment 194: The neuroprotective composition according to any one of embodiments 190-193, wherein said plurality of proteins comprises apolipoprotein E (ApoE), or a biologically active fragment or analog thereof.

Embodiment 195: The neuroprotective composition according to any one of embodiments 190-194, wherein said plurality of proteins comprises secreted frizzled related protein 1, or a biologically active fragment or analog thereof.

Embodiment 196: The neuroprotective composition according to any one of embodiments 190-195, wherein said plurality of proteins comprises serpin family A member 5, or a biologically active fragment or analog thereof.

Embodiment 197: The neuroprotective composition according to any one of embodiments 190-196, wherein said plurality of proteins comprises thrombospondin 4, or a biologically active fragment or analog thereof.

Embodiment 198: The neuroprotective composition according to any one of embodiments 190-197, wherein said plurality of proteins comprises vascular endothelial growth factor B, or a biologically active fragment or analog thereof.

Embodiment 199: The neuroprotective composition according to any one of embodiments 190-198, wherein said plurality of proteins comprises fibroblast growth factor 11 (FGF11), or a biologically active fragment or analog thereof.

Embodiment 200: The neuroprotective composition according to any one of embodiments 190-199, wherein said plurality of proteins comprises fibroblast growth factor 19 (FGF19), or a biologically active fragment or analog thereof.

Embodiment 201: The neuroprotective composition according to any one of embodiments 182-200, wherein said plurality of proteins comprise or consists of one or more proteins selected from the group consisting of ANG-1, ANG, IGFBP-2, TIMP-1, SERPINA5, FGF19, and ApoE, or biologically active fragments or analogs thereof.

Embodiment 202: The neuroprotective composition of embodiment 201, wherein said plurality of proteins comprises ANG-1, or a biologically active fragment or analog thereof.

Embodiment 203: The neuroprotective composition according to any one of embodiments 201-202, wherein said plurality of proteins comprises ANG, or a biologically active fragment or analog thereof.

Embodiment 204: The neuroprotective composition according to any one of embodiments 201-203, wherein said plurality of proteins comprises IGFBP-2, or a biologically active fragment or analog thereof.

Embodiment 205: The neuroprotective composition according to any one of embodiments 201-204, wherein said plurality of proteins comprises TIMP-1, or a biologically active fragment or analog thereof.

Embodiment 206: The neuroprotective composition according to any one of embodiments 201-205, wherein said plurality of proteins comprises SERPINA5, or a biologically active fragment or analog thereof.

Embodiment 207: The neuroprotective composition according to any one of embodiments 201-206, wherein said plurality of proteins comprises FGF19, or a biologically active fragment or analog thereof.

Embodiment 208: The neuroprotective composition according to any one of embodiments 201-207, wherein said plurality of proteins comprises ApoE, or a biologically active fragment or analog thereof.

Embodiment 209: The neuroprotective composition according to any one of embodiments 182-189, wherein said plurality of proteins comprises two or more proteins shown in Table 9, or biologically active fragments or analogs thereof.

Embodiment 210: The neuroprotective composition of embodiment 209, wherein said two or more proteins comprise Aldolase C.

Embodiment 211: The neuroprotective composition according to any one of embodiments 209-210, wherein said two or more proteins comprise Alpha Lactalbumin.

Embodiment 212: The neuroprotective composition according to any one of embodiments 209-211, wherein said two or more proteins comprise ANGPTL3.

Embodiment 213: The neuroprotective composition according to any one of embodiments 209-212, wherein said two or more proteins comprise ApoA2.

Embodiment 214: The neuroprotective composition according to any one of embodiments 209-213, wherein said two or more proteins comprise ApoE.

Embodiment 215: The neuroprotective composition according to any one of embodiments 209-214, wherein said two or more proteins comprise BCAM.

Embodiment 216: The neuroprotective composition according to any one of embodiments 209-215, wherein said two or more proteins comprise MUC1.

Embodiment 217: The neuroprotective composition according to any one of embodiments 209-216, wherein said two or more proteins comprise MUC16.

Embodiment 218: The neuroprotective composition according to any one of embodiments 209-217, wherein said two or more proteins comprise Caspase-3.

Embodiment 219: The neuroprotective composition according to any one of embodiments 209-218, wherein said two or more proteins comprise Cathepsin B.

Embodiment 220: The neuroprotective composition according to any one of embodiments 209-219, wherein said two or more proteins comprise CBP.

Embodiment 221: The neuroprotective composition according to any one of embodiments 209-220, wherein said two or more proteins comprise TfR.

Embodiment 222: The neuroprotective composition according to any one of embodiments 209-221, wherein said two or more proteins comprise CEA.

Embodiment 223: The neuroprotective composition according to any one of embodiments 209-222, wherein said two or more proteins comprise Ceruloplasmin.

Embodiment 224: The neuroprotective composition according to any one of embodiments 209-223, wherein said two or more proteins comprise Chemerin.

Embodiment 225: The neuroprotective composition according to any one of embodiments 209-224, wherein said two or more proteins comprise CHI3L1.

Embodiment 226: The neuroprotective composition according to any one of embodiments 209-225, wherein said two or more proteins comprise CK-MB.

Embodiment 227: The neuroprotective composition according to any one of embodiments 209-226, wherein said two or more proteins comprise Clusterin.

Embodiment 228: The neuroprotective composition according to any one of embodiments 209-227, wherein said two or more proteins comprise C2.

Embodiment 229: The neuroprotective composition according to any one of embodiments 209-228, wherein said two or more proteins comprise C5a.

Embodiment 230: The neuroprotective composition according to any one of embodiments 209-229, wherein said two or more proteins comprise Corticosteroid-binding globulin.

Embodiment 231: The neuroprotective composition according to any one of embodiments 209-230, wherein said two or more proteins comprise C-Peptide.

Embodiment 232: The neuroprotective composition according to any one of embodiments 209-231, wherein said two or more proteins comprise Troponin T.

Embodiment 233: The neuroprotective composition according to any one of embodiments 209-232, wherein said two or more proteins comprise Cytokeratin 19.

Embodiment 234: The neuroprotective composition according to any one of embodiments 209-233, wherein said two or more proteins comprise BNP.

Embodiment 235: The neuroprotective composition according to any one of embodiments 209-234, wherein said two or more proteins comprise ACTH.

Embodiment 236: The neuroprotective composition according to any one of embodiments 209-235, wherein said two or more proteins comprise Exostosin-like 2.

Embodiment 237: The neuroprotective composition according to any one of embodiments 209-236, wherein said two or more proteins comprise Ferritin.

Embodiment 238: The neuroprotective composition according to any one of embodiments 209-237, wherein said two or more proteins comprise Fibrinopeptide A.

Embodiment 239: The neuroprotective composition according to any one of embodiments 209-238, wherein said two or more proteins comprise FSH.

Embodiment 240: The neuroprotective composition according to any one of embodiments 209-239, wherein said two or more proteins comprise GLP-1.

Embodiment 241: The neuroprotective composition according to any one of embodiments 209-240, wherein said two or more proteins comprise GMNN.

Embodiment 242: The neuroprotective composition according to any one of embodiments 209-241, wherein said two or more proteins comprise Hemopexin.

Embodiment 243: The neuroprotective composition according to any one of embodiments 209-242, wherein said two or more proteins comprise HSP27.

Embodiment 244: The neuroprotective composition according to any one of embodiments 209-243, wherein said two or more proteins comprise HSP90.

Embodiment 245: The neuroprotective composition according to any one of embodiments 209-244, wherein said two or more proteins comprise IL-34.

Embodiment 246: The neuroprotective composition according to any one of embodiments 209-245, wherein said two or more proteins comprise Kallikrein 2.

Embodiment 247: The neuroprotective composition according to any one of embodiments 209-246, wherein said two or more proteins comprise Kallikrein 10.

Embodiment 248: The neuroprotective composition according to any one of embodiments 209-247, wherein said two or more proteins comprise Lyn.

Embodiment 249: The neuroprotective composition according to any one of embodiments 209-248, wherein said two or more proteins comprise NPTXR.

Embodiment 250: The neuroprotective composition according to any one of embodiments 209-249, wherein said two or more proteins comprise P-Cadherin.

Embodiment 251: The neuroprotective composition according to any one of embodiments 209-250, wherein said two or more proteins comprise PIM2.

Embodiment 252: The neuroprotective composition according to any one of embodiments 209-251, wherein said two or more proteins comprise PPARg2.

Embodiment 253: The neuroprotective composition according to any one of embodiments 209-252, wherein said two or more proteins comprise PR Isoform B.

Embodiment 254: The neuroprotective composition according to any one of embodiments 209-253, wherein said two or more proteins comprise PSA-free.

Embodiment 255: The neuroprotective composition according to any one of embodiments 209-254, wherein said two or more proteins comprise PTPRD.

Embodiment 256: The neuroprotective composition according to any one of embodiments 209-255, wherein said two or more proteins comprise Ret.

Embodiment 257: The neuroprotective composition according to any one of embodiments 209-256, wherein said two or more proteins comprise Serpin A5.

Embodiment 258: The neuroprotective composition according to any one of embodiments 209-257, wherein said two or more proteins comprise SHBG.

Embodiment 259: The neuroprotective composition according to any one of embodiments 209-258, wherein said two or more proteins comprise SOX2.

Embodiment 260: The neuroprotective composition according to any one of embodiments 209-259, wherein said two or more proteins comprise Angiogenin.

Embodiment 261: The neuroprotective composition according to any one of embodiments 209-260, wherein said two or more proteins comprise Angiopoietin-1.

Embodiment 262: The neuroprotective composition according to any one of embodiments 209-261, wherein said two or more proteins comprise CCR5.

Embodiment 263: The neuroprotective composition according to any one of embodiments 209-262, wherein said two or more proteins comprise CD40 Ligand.

Embodiment 264: The neuroprotective composition according to any one of embodiments 209-263, wherein said two or more proteins comprise Chordin-Like-1.

Embodiment 265: The neuroprotective composition according to any one of embodiments 209-264, wherein said two or more proteins comprise Cripto-1.

Embodiment 266: The neuroprotective composition according to any one of embodiments 209-265, wherein said two or more proteins comprise CXCR6.

Embodiment 267: The neuroprotective composition according to any one of embodiments 209-266, wherein said two or more proteins comprise EG-VEGF.

Embodiment 268: The neuroprotective composition according to any one of embodiments 209-267, wherein said two or more proteins comprise ErbB3.

Embodiment 269: The neuroprotective composition according to any one of embodiments 209-268, wherein said two or more proteins comprise FGF-11.

Embodiment 270: The neuroprotective composition according to any one of embodiments 209-269, wherein said two or more proteins comprise FGF-19.

Embodiment 271: The neuroprotective composition according to any one of embodiments 209-270, wherein said two or more proteins comprise GDF-9.

Embodiment 272: The neuroprotective composition according to any one of embodiments 209-271, wherein said two or more proteins comprise GDF-11.

Embodiment 273: The neuroprotective composition according to any one of embodiments 209-272, wherein said two or more proteins comprise Granzyme A.

Embodiment 274: The neuroprotective composition according to any one of embodiments 209-273, wherein said two or more proteins comprise GRO.

Embodiment 275: The neuroprotective composition according to any one of embodiments 209-274, wherein said two or more proteins comprise HCR.

Embodiment 276: The neuroprotective composition according to any one of embodiments 209-275, wherein said two or more proteins comprise NRG1 Isoform GGF2.

Embodiment 277: The neuroprotective composition according to any one of embodiments 209-276, wherein said two or more proteins comprise IFN-alpha/beta R2.

Embodiment 278: The neuroprotective composition according to any one of embodiments 209-277, wherein said two or more proteins comprise IGFBP-2.

Embodiment 279: The neuroprotective composition according to any one of embodiments 209-278, wherein said two or more proteins comprise IL-38.

Embodiment 280: The neuroprotective composition according to any one of embodiments 209-279, wherein said two or more proteins comprise IL-7.

Embodiment 281: The neuroprotective composition according to any one of embodiments 209-280, wherein said two or more proteins comprise IL-17 RB.

Embodiment 282: The neuroprotective composition according to any one of embodiments 209-281, wherein said two or more proteins comprise IL-17 RD.

Embodiment 283: The neuroprotective composition according to any one of embodiments 209-282, wherein said two or more proteins comprise IL-21.

Embodiment 284: The neuroprotective composition according to any one of embodiments 209-283, wherein said two or more proteins comprise IL-23 R.

Embodiment 285: The neuroprotective composition according to any one of embodiments 209-284, wherein said two or more proteins comprise IL-26.

Embodiment 286: The neuroprotective composition according to any one of embodiments 209-285, wherein said two or more proteins comprise IL-29.

Embodiment 287: The neuroprotective composition according to any one of embodiments 209-286, wherein said two or more proteins comprise Insulysin.

Embodiment 288: The neuroprotective composition according to any one of embodiments 209-287, wherein said two or more proteins comprise Kremen-2.

Embodiment 289: The neuroprotective composition according to any one of embodiments 209-288, wherein said two or more proteins comprise MFRP.

Embodiment 290: The neuroprotective composition according to any one of embodiments 209-289, wherein said two or more proteins comprise MIF.

Embodiment 291: The neuroprotective composition according to any one of embodiments 209-290, wherein said two or more proteins comprise MIP 2.

Embodiment 292: The neuroprotective composition according to any one of embodiments 209-291, wherein said two or more proteins comprise MMP-9.

Embodiment 293: The neuroprotective composition according to any one of embodiments 209-292, wherein said two or more proteins comprise MMP-16.

Embodiment 294: The neuroprotective composition according to any one of embodiments 209-293, wherein said two or more proteins comprise MMP-25.

Embodiment 295: The neuroprotective composition according to any one of embodiments 209-294, wherein said two or more proteins comprise NRG1 Isoform GGF2.

Embodiment 296: The neuroprotective composition according to any one of embodiments 209-295, wherein said two or more proteins comprise Orexin-A.

Embodiment 297: The neuroprotective composition according to any one of embodiments 209-296, wherein said two or more proteins comprise Oncostatin M.

Embodiment 298: The neuroprotective composition according to any one of embodiments 209-297, wherein said two or more proteins comprise OX40 Ligand.

Embodiment 299: The neuroprotective composition according to any one of embodiments 209-298, wherein said two or more proteins comprise PDGF-AA.

Embodiment 300: The neuroprotective composition according to any one of embodiments 209-299, wherein said two or more proteins comprise PDGF-C.

Embodiment 301: The neuroprotective composition according to any one of embodiments 209-300, wherein said two or more proteins comprise ROBO4.

Embodiment 302: The neuroprotective composition according to any one of embodiments 209-301, wherein said two or more proteins comprise sFRP-1.

Embodiment 303: The neuroprotective composition according to any one of embodiments 209-302, wherein said two or more proteins comprise SIGIRR.

Embodiment 304: The neuroprotective composition according to any one of embodiments 209-303, wherein said two or more proteins comprise SMAD1.

Embodiment 305: The neuroprotective composition according to any one of embodiments 209-304, wherein said two or more proteins comprise SMAD4.

Embodiment 306: The neuroprotective composition according to any one of embodiments 209-305, wherein said two or more proteins comprise SMAD5.

Embodiment 307: The neuroprotective composition according to any one of embodiments 209-306, wherein said two or more proteins comprise SMAD7.

Embodiment 308: The neuroprotective composition according to any one of embodiments 209-307, wherein said two or more proteins comprise Spinesin.

Embodiment 309: The neuroprotective composition according to any one of embodiments 209-308, wherein said two or more proteins comprise TACI.

Embodiment 310: The neuroprotective composition according to any one of embodiments 209-309, wherein said two or more proteins comprise Thrombospondin-4.

Embodiment 311: The neuroprotective composition according to any one of embodiments 209-310, wherein said two or more proteins comprise TIMP-1.

Embodiment 312: The neuroprotective composition according to any one of embodiments 209-311, wherein said two or more proteins comprise TLR1.

Embodiment 313: The neuroprotective composition according to any one of embodiments 209-312, wherein said two or more proteins comprise TRADD.

Embodiment 314: The neuroprotective composition according to any one of embodiments 209-313, wherein said two or more proteins comprise TWEAK.

Embodiment 315: The neuroprotective composition according to any one of embodiments 209-314, wherein said two or more proteins comprise VEGF-B.

Embodiment 316: The neuroprotective composition according to any one of embodiments 190-315, wherein said protein(s) comprise full-length proteins.

Embodiment 317: The neuroprotective composition according to any one of embodiments 190-315, wherein said protein(s) comprise biologically active fragments of said proteins.

Embodiment 318: The neuroprotective composition according to any one of embodiments 190-315, wherein said protein(s) comprise analogs of said proteins.

Embodiment 319: The neuroprotective composition according to any one of embodiments 190-318, wherein said composition is effective to reduce neuronal apoptosis as compared to an untreated control.

Embodiment 320: The neuroprotective composition according to any one of embodiments 190-319, wherein said composition is effective to diminish aggregation of TDP43 as compared to an untreated control.

Embodiment 321: The neuroprotective composition according to any one of embodiments 190-320, wherein said composition is effective to diminish neurite shrinkage as compared to an untreated control.

Embodiment 322: The neuroprotective composition according to any one of embodiments 190-321, wherein said composition comprises a pharmaceutically acceptable carrier and is a pharmaceutical formulation.

Embodiment 323: The neuroprotective composition of embodiment 322, wherein said composition comprises a substantially sterile pharmaceutical formulation.

Embodiment 324: The neuroprotective composition according to any one of embodiments 322-323, wherein pharmaceutical formulation is formulated for administration to a mammal via a route selected from the group consisting of oral delivery, isophoretic delivery, transdermal delivery, parenteral delivery, aerosol administration, administration via inhalation, intravenous administration, and rectal administration.

Embodiment 325: The neuroprotective composition according to any one of embodiments 322-323, wherein pharmaceutical formulation is formulated for administration to the brain or spinal cord of a mammal.

Embodiment 326: The neuroprotective composition according to any one of embodiments 322-323, wherein pharmaceutical formulation is formulated for intracerebral, ventricular or intrathecal delivery to a mammal.

Embodiment 327: The neuroprotective composition according to any one of embodiments 322-323, wherein pharmaceutical formulation is formulated for intranasal delivery to a mammal.

Embodiment 328: The neuroprotective composition according to any one of embodiments 322-323, wherein pharmaceutical formulation is formulated for delivery via an implant in a mammal.

Embodiment 329: The neuroprotective composition according to any one of embodiment 182-328, wherein said composition is effective to inhibit apoptosis, and/or to attenuate oxidative stress, and/or to repair oxidative damage, and/or prevent protein aggregation and mislocalization, and/or prevent loss of axonal, etc. neuron projections, and/or prevent denervation, and/or increase innervation, muscle mass, neuro-muscular function, prevent and attenuate paralysis and morbidity when administered to a mammal.

### Definitions

The terms “subject,” “individual,” and “patient” may be used interchangeably and refer to humans, as well as non-human mammals (e.g., non-human primates, canines, equines, felines, rodents, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein, the phrase “a subject in need thereof” refers to a subject, as described infra, that suffers from, or is at risk for a pathology characterized by neurodegeneration (e.g., oxidative stress induced neuronal cell death). Thus, for example, in certain embodiments the subject is a subject with, or at risk for a motor neuron disease, Alzheimer's disease, Parkinson's disease, Huntington's disease, HIV-1 associated neurological degeneration, neurodegeneration associated with an ischemic event, neurodegeneration associated with traumatic brain injury (TBI), neurodegeneration associated with a spinal cord injury, drug-induced neurodegeneration, cancer-associated neurodegeneration, glaucoma, and the like.

The term “treat” when used with reference to treating, e.g., a pathology or disease refers to the mitigation and/or elimination of one or more symptoms of that pathology or disease, and/or a delay in the progression and/or a reduction in the rate of onset or severity of one or more symptoms of that pathology or disease, and/or the prevention of that pathology or disease. The term treat can refer to prophylactic treatment which includes a delay in the onset or the prevention of the onset of a pathology or disease.

By “embryonic stem cell” or “ES cell” it is meant a cell that a) can self-renew, b) can differentiate to produce all types of cells in an organism, and c) is derived from a developing organism or is an established ES cell line which was derived from a developing organism. ES cells may be derived from the inner cell mass of the blastula of a developing organism. ES cells may be derived from a blastomere generated by single blastomere biopsy (SBB) involving removal of a single blastomere from the eight cell stage of a developing organism. In general, SBB provides a non-destructive alternative to inner cell mass isolation. SBB and generation of hES cells from the biopsied blastomere is described by Chung et al. (2008) Cell Stem Cell, 2(2):113-117. ES cells can be cultured over a long period of time while maintaining the ability to differentiate into all types of cells in an organism. In culture, ES cells typically grow as flat colonies with large nucleo-cytoplasmic ratios, defined borders and prominent nuclei. In addition, ES cells express SSEA-3, SSEA-4, TRA-1-60, TRA-1-81, and Alkaline Phosphatase, but not SSEA-1. Examples of methods of generating and characterizing ES cells may be found in, for example, U.S. Pat. Nos. 7,029,913, 5,843,780, and 6,200,806, the disclosures of which are incorporated herein by reference for methods of generating and characterizing ES cells described therein.

By “pluripotent stem cell” or “PSC cell” it is meant a cell that a) can self-renew, b) can differentiate to produce all types of cells that are derivatives of any of the three germinal layers (endoderm, mesoderm, and ectoderm), and c) is derived from a developing organism (embryonic stem cell, ESC) or is an induced pluripotent stem cell that is derived from somatic cells through Yamanaka factors reprogramming (Cell 2006, Cell 2007). PS cells can be cultured over a long period of time while maintaining the ability to differentiate into all types of cells in an organism. In culture, PS cells typically grow as flat colonies with large nucleo-cytoplasmic ratios, defined borders and prominent nuclei. In addition, PS cells express Oct4, Sox-2, Nanog, Lin 28, SSEA-3, SSEA-4, TRA-1-60, TRA-1-81, and Alkaline Phosphatase. Examples of methods of generating and characterizing PS cells are well known to those of skill in the art, and may be found in, for example, in PCT Patent Pub. No: WO2016120493A1, in U.S. Pat. Nos. 8,642,334, 8,628,963, 8,603,818, 8,551,472, 8,546,140, 8,535,944, 8,530,238, 8,518,700, 8,515,150, 8,507,274, 8,496,941, 8,481,492, 8,420,352, 8,372,642, 8,349,609, 8,323,971, 8,298,825, 8,278,104, 8,268,621, 8,257,941, 8,211,697, 8,183,297, 8,129,187, 8,058,065, 8,048,999, 8,048,675, 7,410,797, 7,280,534, 7,150,990, 6,808,392, 6,703,017, 6,007,993, and the like. PS cells may be from any organism of interest, including, primate, e.g., human; canine; feline; murine; equine; porcine; avian; camel; bovine; ovine, and so on.

A “pharmaceutically acceptable carrier” means carrier that is useful in preparing a pharmaceutical composition that is generally safe, non-toxic and neither biologically nor otherwise undesirable, and includes carriers that are acceptable for veterinary use as well as human pharmaceutical use.

As used herein, a “pharmaceutical composition” is meant to encompass a composition suitable for administration to a subject, such as a mammal, especially a human. A “pharmaceutical composition” may be sterile and free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration.

The terms “biologically active fragment” when used with respect to a protein refers to a fragment of that protein that possess the same biological activity as the full-length protein. In certain embodiments, the biological fragment provides at least 75%, or at least 80%, or at least 85%, or at least 90%, or at least 95%, or at least 98%, or at least 99%, or 100% of the activity level provided by the full-length protein.

The phrase “contacting a cell with proteins secreted by . . . cells” refers to the actual proteins obtained directly from the secretome of the reference cells or contacting cells with proteins that are the same as the protein(s) identified from the secretome but that may be obtained from other sources. Additionally, contacting cells with biologically active fragments of the proteins or biologically active analogs of the proteins is contemplated.

### BRIEF DESCRIPTION OF THE DRAWINGS

FIG. 1, panels A-D, shows that human embryonic stem cell-derived conditioned medium (hESC-CM) enhances viability of human fibroblast that are exposed to H₂O₂. Panel A) The viability of IMR90 fibroblasts was examined with different concentrations of H₂O₂. Panel B) The viability of IMR90 was examined with different concentrations of hESC-CM. Panel C) MT viability assay of IMR90 cells at 200 μM H₂O₂ and indicated culture conditions. Panel D) Flow Cytometry assay and the dot-plot analyses of IMR90 cells that were treated with 200 μM H₂O₂ alone or co-treated with hESC-CM or dF-CM. Bar graphs with Means and SEM show quantification of triplicate MTT and triplicate Flow Cytometry assays for each experimental condition. *P<0.05, **P<0.01 ***P<0.001.

FIG. 2, panels A-E, shows that hESC-CM protects human motor neurons (MNs) from H₂O₂ cytotoxicity. Panel A) MTT assay for viability of MNs that were cultured with different concentrations of H₂O₂. Panel B) MTT assay for viability of MNs that were cultured with different concentrations of hESC-CM. Panel C) MTT assay for viability of MNs that were cultured with 200 μM H₂O₂ and indicated media and CMs. Panel D) Annexin V apoptotic index of MNs exposed to 200 μM H₂O₂ in indicated culture conditions (fold of the first replicate of Control). Annexin V fluorescence was assayed by the SpectraMax iD3 microplate reader. Panel E) The time course of apoptosis of MNs that were pre-treated with hESC-CM or post-treated (H₂O₂ first, then hESC-CM); the ratios or apoptotic to live cells were determined by Annexin V using SpectraMax iD3 microplate reader. *P<0.05, **P<0.01 ***P<0.001. Triplicate MTT and Annexin V assays were performed.

FIG. 3, panels A-I, shows that ALS iPSC conditioned medium is neuroprotective for ALS-MNs. Panel A) The viability of ALS-MNs exposed to H₂O₂ was measured by MTT assay in triplicates. Panel B) The ratios of apoptotic to live ALS-MNs were assayed by Annexin V fluorescence in a plate reader in triplicates. Panel C) Representative images of MNs at 30 days and 60 days: WT—WT iPSC differentiated into MNs, ALS CS53-ALS iPSC differentiated into MNs, ALS CS53+CM—iPSC differentiated into MNs in the presence of ALS iPSC conditioned medium. Scale bar, 200 μm. Panel D) The neurite sizes of wild type and ALS-MNs were measured during cell differentiation from hESC, iPSC and ALS-iPSCs. Triplicate differentiating MN cultures were analyzed for each genotype and condition, at each time point. Panel E) The neurite sizes were measured in ALS-MNs that were treated with autologous ALS-CM at different time points of their differentiation. Panel F) The net change of neurite size was examined during differentiation of ALS-MNs that were treated with autologous ALS iPSC-CM, as indicated. Triplicate differentiating MN cultures of each genotype and condition were analyzed at each time point. Panel G) The percent of activated Caspase3 positive cells was quantified during this directed differentiation of MNs and compared between control cells and treated with autologous ALS-CM in triplicate cultures for each cell line, condition and time point. Panel H) Representative images of TDP-43 expression in MNPs. The cytoplasmic TDP-43 increases in H₂O₂ group as compared to Control and ALS PSC-CM, but less so in the ALS dF-CM. Panel I) Representative Western blots and densitometric quantification of subcellular distribution of TDP-43 from five independent Western Blotting experiments. Values are mean±SEM, N=5. *P<0.05, **P<0.01, ***P<0.001.

FIG. 4, panels A-H, illustrates comparative proteomics and fractionation of PSC-CM. Panel A) MTT assay for viability of MNs that were cultured with different concentrations of HBP fraction of hESC-CM. Quadruplicate assays for each condition. Panel B) MTT assay for viability of MNs that were cultured with different concentrations of exosome fraction of hESC-CM. Triplicate assays for each condition. Panel C) MTT assay for viability of MNs that were cultured with 200 μg/ml H₂O₂ alone or co-treated with complete unfractionated hESC-CM, HBP fraction, exosome fraction or HPB flow through and exosome-free CM. *P<0.05, **P<0.01, Quadruplicates for each condition. Panel D) The Venn diagram of the proteins that are present at different levels in studied conditioned media. Panel E) Violin plot of cohort distributions. Panel F) Heat mapping of the differentially present proteins that are grouped on their key indicated biological functions. Panel G) Heat mapping of Heparin binding proteins and neuroprotective proteins. Panel H) A mechanism of multifunctional neuroprotection through PSC-CM. Oxidative damage alters SOD1 activity, and dysfunctional or mutant SOD1 causes further oxidative damage; this induces Cas3, apoptosis and TDP43 mislocalization—aggregation. The latter feeds back to perturb SOD1, and also promotes apoptosis. PSC-CM proteins have capacity to act at multiple stages of SOD1-TDP43-Cas3 deregulation: lowering ROS, attenuating TDP43 aggregation and improving proteostasis, inhibiting Cas3 activation and apoptosis.

FIG. 5, panels A-E, illustrates the effect of the secretome from ALS iPSCs on SOD1^{G93A} Transgenic mice. Panel A) The Hanging test. The latency in ALS iPSC-CM group (Male, N=3 and Female, N=3) was greater than dF-CM (Male, N=2 and Female, N=2). Panel B) The onset time of symptoms. Kaplan-Meier curves of the disease onset showing that the onset time in ALS iPSC-CM group was delayed compared to that in dF-CM group (ALS iPSC group, N=6 and dF-CM group, N=4). The bar graph shows the average time of the disease onset between two groups. Panel C) The survival time. Kaplan-Meier curves of the survival showing that the secretome from ALS iPSC-CM increased the lifespan compared to that from dF-CM (ALS iPSC group, N=6 and dF-CM group, N=4). The bar graph shows the average time of the survival between two groups. Panel D) Cresyl violet staining in the ventral lumbar spinal cord at 120 days. After the treatment of ALS iPSC-CM, the number and morphology of motor neurons improved compared to that of dF-CM. The bar graph shows the difference in the number of motor neurons among groups. The arrow head points to shrunk stained MNs. Scale bar, 200 μm. Panel E) Immunostaining of NMJ in gastrocnemius muscle at 120 days. The colocalized endplates shows NMJ innervation. The bar graph shows that the number of colocalized NMJ decreased in the dF-CM groups compared to the WT, whereas the number was maintained highly in ALS iPSC-CM group compared to the dF-CM group. The arrow indicates the colocalized endplates and the arrow head points to discolocalized endplates. NF=Neurofilament, SYN=Synaptophysin, AchR=Acetylcholine receptors, Scale bar, 100 μm. Values are mean±SEM, * P<0.05, **P<0.01.

FIG. 6 illustrates the morphologies of IMR90 cells that were cultured with/without H₂O₂, co-treated with hESC-CM or df-CM. Scale bar, 100 μm.

FIG. 7 shows that antioxidant activity is not different between human embryonic stem cell-derived conditioned medium (hESC-CM) and differentiated fibroblast-derived conditioned medium (dF-CM). Each CM was collected after culturing of respective cells in OptiMEM for 24 hours. Antioxidant activity assay (BioAssay systems, DTAC-100 kit) was performed in triplicate wells for each hESC-CM, df-CM and the control medium, OptiMeM (optMEM), as recommended by the manufacturer. The antioxidant-reporting fluorescent signal intensity was measured by microplate reader (570 nm). The data are expressed as fold of the Mean of OptiMEM control, which set at 1.

FIG. 8, panels A-B, illustrates differentiation of PSCs into motor neurons (MNs). Panel A) The expression of stage specific markers for pluripotency (OCT4 and SOX2), motor neuron precursor (OLIG2), and MNs (TUJ1, BH9 and CHAT) by immunofluorescence; DAPI labels all nuclei. Scale bar, 100 μm. Panel B) Gene expression profiles of stage specific markers during differentiation into MNs by qRT-PCR that was performed in 3 replicates for each gene. ***P<0.001. After the motor-neuron identity and sufficient (above 90%) enrichment of these cultures were established, these human motor neurons were used in subsequent experiments. OLIG2 was detected at 14 days post differentiation. HB9, a specific marker for MNs, was observed at 30 days. Additionally, HB9 and CHAT gene expression were expressed at 30 days and as expected, these were lacking in the undifferentiated PSCs. In concert, the expression of pluripotency-related genes decreased dramatically upon the directed differentiation into the motor neuron lineage.

FIG. 9, panels A-B, illustrates differentiation of ALS iPSCs into motor neurons (ALS-MNs). Panel A) The expression of cell-fate markers for pluripotency (OCT4 and SOX2), motor neuron precursors (OLIG2), and MNs (TUJ1, BH9 and CHAT) by immunofluorescence; DAPI labels all nuclei. Scale bar, 100 μm. Panel B) qRT-PCR gene expression in 3 replicates for each gene assayed for the indicated cell-fate markers during differentiation of ALS-iPCSs into ALS-MNs ***P<0.001.

FIG. 10, panels A-E, illustrates the comparative effects of autologous ALS-conditioned media (ALS-CM) on neurites and apoptosis of ALS-motor neurons (ALS-MNs) in two independent ALS cell patients' cell lines. Panel A) The net change of neurite size was calculated in these differentiating MNs. Panel B) The time-course of the conditioned medium treatment. Panel C) Representative images of motor neurons with their neurites (CS07-ALS donor), (CS07-CM ALS donor iPSC conditioned medium) at 30 days and 60 days. Scale bar, 200 μm. Panel D) The neurite sizes were measured in ALS-MNs that were treated with autologous ALS-CM at different time points of their differentiation. Panel E) Representative images of apoptotic Caspase 3 positive MNs at 30 days and 60 days of their directed differentiations from the wt and ALS iPSCs. Caspase 3 (green) was immunodetected with specific antibody, DAPI, blue stains all nuclei. Scale bar, 100 μm.

FIG. 11 shows original images of representative Western Blots. Shown are the TDP43, PCNA, O-Actin immune-detected bands and their predicted sizes, as per the MW ladders. Faint non-specific bands are also shown.

FIG. 12 shows quantification of apoptotic MNPs after H₂O₂ alone as compared to co-treatment with ALS iPSC-CM or dF-CM. Values are mean±SEM, *P<0.05, **P<0.01, N=triplicate assays for each condition.

FIG. 13 shows the top ten differentially present proteins in each of the listed categories. In the CC group, the top 10 most significantly up-regulated terms contained extracellular space, extracellular region, extracellular exosome, cell surface, blood microparticle, platelet alpha granule lumen, Golgi lumen, SMAD protein complex, membrane raft, and external side of plasma membrane. The top 10 most significantly up-regulated terms in the MF group comprised growth factor activity, cytokine activity, receptor binding, collagen binding, heparin binding, serine-type endopeptidase activity, tumor necrosis factor receptor binding, Ras guanyl-nucleotide exchange factor activity, protein homodimerization activity, and identical protein binding.

FIG. 14 illustrates results of the protein cocktail test. To investigate the effect of the cocktail, four recombinant proteins, TIMP1, Angiogenin, Angiopoietin-1 and Apolipoprotein E were mixed and cultured with damaged MNs by H₂O₂. However, there was no difference between cocktail group and only H₂O₂ group.

FIG. 15, panels A-B, illustrates the effect of CMs on the weights and neurological score in SOD1^{G93A} transgenic mice. Panel A) The change of weights. There was no significant difference between ALS iPSC-CM group (Male, N=3 and Female, N=3) and dF-CM group (Male, N=2 and Female, N=2). Panel B) The effect on the neurological score. These data were used to check the health condition of mice and to determine the survival rate and the end-stage of the ALS iPSC-CM group (Male, N=3 and Female, N=3) and the dF-CM group (Male, N=2 and Female, N=2).

FIG. 16, panels a-g, shows that ALS iPSC conditioned medium (CM) is neuroprotective for ALS-MNs. Panel a) The effect of ALS iPSC-CM on viability and apoptosis in ALS-MNs. The viability of ALS-MNs exposed to H₂O₂ was measured by MTT assay. The ratios of apoptotic to live ALS-MNs were assayed by Annexin V fluorescence in a plate reader. N=4, *P<0.05. Panel b) The time course of viability (560/590 nm) and apoptosis (490/590 nm) of MNs that were pre-treated with ALS iPSC-CM or post-treated (H₂O₂ first, then ALS iPSC-CM). Panel c) Representative images of MNs at 30 days and 60 days: WT—WT iPSC differentiated into MNs, ALS CS53-ALS iPSC differentiated into MNs, ALS CS53+CM—iPSC differentiated into MNs in the presence of ALS iPSC conditioned medium. Scale bar, 200 μm. Panel d) Experimental schematic. The CMs from WT-iPSC, ALS-iPSC, and from iPSCs-derived fibroblasts (dF-CM) were used to treat the differentiating MN cultures on days 30 to 60, after which MN assays were performed. Panel e) The neurite sizes were measured during MN differentiation from hESC, iPSC and ALS-iPSCs (+/−autologous ALS iPSC-CM at d30, 35, 40, 45, and d50). ALS-MNs neurites of ALS-MNs (CS53, CS07) were maintained in the +ALS iPSC-CM cultures when treatment started on d30 or d35, but not on later days. For the negative control, fibroblast-CM (dF-CM), there was no cells that maintained neurites. Graphs show Mean±SEM, N=4 independent MN differentiation study with each cell line. *P<0.05. Panel f) The net change of neurite size was examined during differentiation of ALS-MNs that were treated with autologous ALS iPSC-CM, as indicated. Panel g) The percent of activated Caspase3 positive cells was quantified during this directed differentiation of MNs and compared between control cells and treated with autologous ALS-CM. Representative images of apoptotic Caspase 3 positive MNs are shown. Caspase 3 (green) was immunodetected with specific antibody, DAPI, blue stains all nuclei. Graphs show Mean±SEM, N=5. *P<0.05, **P<0.01, ***P<0.001. Scale bar, 100 μm.

FIG. 17, panels a-g, illustrates in vivo neuroprotection of ALS iPSCs secretome in the mouse ALS model, SOD1^{G93A}. Panel a) Hanging test of the transgenic mice (ALS iPSC-CM treated mice N=12 and dF-CM treated mice N=6). The decline in performance was significantly delayed in the mice treated with ALS iPSCs-CM, as compared to the dF-CM (114.8±4.29 days vs. 91.0±4.31 days, P<0.01). Values are mean±SEM. Panel b) Kaplan-Meier curves of the disease onset showing that onset was delayed in the ALS iPSC-CM, as compared to the dF-CM group. Onset: (116.8±3.64 days vs. 92.3±3.44 days, P<0.001), consistent with the neurological scoring that is shown in FIG. 2, panel d. Panel c) Kaplan-Meier curves of animal survival show that the ALS iPSC-CM increased lifespan, as compared to dF-CM (141.5±3.26 days vs. 120.3±3.07 days, P<0.001). Panel d) Neurological scores were analyzed as in Methods in Example 2 and were found to be improved by the ALS iPSC-CM, as compared to dF-CM. Panel e) Histological analysis (WT for SOD1 C57.B6 mice, N=6, and ALS mice, N=8) at 120 days of age, and at the end stage (morbidity time point). Representative images for Cresyl violet staining (and their quantification, bar graph) in the ventral lumbar spinal cord at 120 days show that ALS iPSC-CM improved the number and morphology of motor neurons of ALS mice, as compared to dF-CM. The arrowhead points to shrunken MNs. Scale bar, 200 μm. Panel f) Representative images of immunostaining of NMJ in gastrocnemius muscle at 120 days and the bar graph quantification show that the number of colocalized NMJ decreased in the dF-CM group, as compared to the WT mice, whereas this number was maintained better in the ALS iPSC-CM group. The arrow indicates the colocalized endplates and the arrowhead points to mis-localized endplates. NF=Neurofilament, SYN=Synaptophysin, AchR=Acetylcholine receptors, Scale bar, 100 μm. Panel g) Mass of tibialis anterior (TA) and gastrocnemius (GA) muscles was also increased in the ALS mice that were treated with ALS iPSC-CM as compared to df-CM.

FIG. 18, panels a-g, illustrates mechanisms of neuroprotection by ALS iPSC-CM. Panel a) MTT assay for viability of MNs that were cultured with proteinase K treated or heat inactivated ALS iPSC-CM. ***P<0.001. N=4 for each condition. Panel b) MTT assay for viability of MNs that were cultured with 200 μg/ml H₂O₂ alone or co-treated with complete unfractionated ALS iPSC-CM, HBP fraction, exosome fraction or HPB flow through and exosome-free CM. *P<0.05, **P<0.01, N=4 for each condition. Panel c) MTT assay for viability of MNs that were cultured with secretome of ALS iPSC-derived embryoid bodies (EBs). The viability was significantly decreased in EB-CM group compared to it in ALS iPSC-CM group (0.66±0.02 vs. 0.44±0.05, P<0.001). ***P<0.001. N=4 for each condition. Panel d) The effect of epigenetic modifiers on the neuroprotective activity of the iPSC secretome: ALS iPSCs were treated with the indicated inhibitors of pluripotency that influence epigenetics, and the activity of their CM was subsequently tested with H₂O₂ exposed ALS-MNPs, in quadruplicate MTT and Annexin V assays. EZH2 inhibitor, GSK126 diminished the neuroprotective activity of the iPSC CM with high statistical significance (viability: 0.64±0.04 vs. 0.47±0.02, P<0.01; apoptosis: 1.71±0.17 vs. 2.65±0.23, P<0.01), *P<0.05, **P<0.01, ***P<0.001, N=4 for each condition. Panel e) Levels of mitochondrial ROS. All MNs were stained with mitoSOX red and then the mean fluorescence intensity (MFI) was measured by a Guava Easycyte Flowcytometer. Autologous ALS iPSC-CM normalized mitochondrial activity of ALS MNs statistically same as the known modifier, Cyclosporin A (CsA). Graphs show Mean±SEM, N=4. **P<0.01. Panel f) The effects on inflammation was assayed by qRT PCR. The indicated markers of inflammation were diminished by ALS iPSC-CM similarly to CsA. N=3. ***P<0.001. ns-not statistically significant. N=4 **P<0.01, ***P<0.001. ns-not statistically significant. Panel g) The effect of CsA on MN viability and apoptosis. The viability of ALS MNs as compared to healthy WTC-11 MNs was measured by MTT assay. The ratios of apoptotic to live MNs were assayed by Annexin V fluorescence in a plate reader. ALS MNs are prone to cell death, as compared to WTC-11 MNs; ALS iPSC-CM, but not dF-CM or CsA, protected ALS MNs from the ROS (mutant SOD1) induced cell death.

FIG. 19, panels a-h, illustrates proteomic profiling analysis. Panel a) Heat mapping of all the groups, WTC11, H9, CS07, CS53, and dF-CM. The dF-CM is farthest from the other groups. Panel b) The Venn diagram of the proteins that are present at different levels in studied conditioned media. Panel c) The KEGG pathway list that has groups on their key indicated biological functions. Panel d) Heat mapping of representative KEGG pathways, iP3K-AKT signaling pathway (P=5.00E-04) and TGF-b signaling pathway (P=9.30E-03). Panel e) Heat mapping of negative regulators of apoptotic process (P=3.00E-06) and inflammatory response (P=2.60E-06). * Negative regulation of neuronal apoptosis (8.00E-03). Panel f) Heat mapping of inflammation related proteins. The levels in PSC-CM are lower than in dF-CM. Panel g) Heat mapping of Heparin bound proteins and neuroprotective proteins. Panel h) Volcano plot of the PSC-CM and dF-CM. The red dots represent differently expressed proteins (P<0.05; FC>1.75), while the grey dots represent proteins with P>0.05.

FIG. 20, illustrates mechanisms of multifunctional neuroprotection through PSC-CM. Oxidative stress alters SOD1 activity, and dysfunctional or mutant SOD1 causes further oxidative damage; this induces apoptosis and destabilizing mitochondria and promoting hyperinflammation. Distinct PSC-CM proteins through different mechanisms have capacity to act at attenuating ROS, improving proteostasis, inhibiting Cas3 activation and apoptosis, promoting neuronal repair. The key candidates in PSC-CM as neuroprotective factors are TIMP1, ApoE, ANG, ANG1, and HSP27, whose neuroprotective effects were confirmed through previous literature. Ultimately, PSC-CM delays or inhibits death and denervation of MNs, promoting better muscle innervation and preventing degeneration. Figure created using BioRender (//biorender.com/).

FIG. 21, panels a-e, shows that human embryonic stem cell-derived conditioned medium (hESC-CM) protects human fibroblast from H₂O₂ cytotoxicity. Panel a) The viability of IMR90 fibroblasts was examined with different concentrations of H₂O₂ and with different concentrations of hESC-CM. Cell viability decreased significantly (below 0.5 value) by 200 μM H₂O₂ treatment. Viability was not affected by up to 30% hESC-CM. Data were normalized to the values at the 0 concentration. Panel b) MTT viability assay of IMR90 cells. Cell viability was noticeably lower in cultures treated with H₂O₂ and those co-treated with H₂O₂ plus the dF-CM as compared to the H₂O₂ plus hESC-CM cultures. The single treatment of each medium was presented as negative controls. Panel c) The morphologies of IMR90 cells that were cultured with/without H₂O₂, co-treated with hESC-CM or dF-CM. Scale bar, 100 μm. Panel d) Flow Cytometry dot-plot analyses of IMR90 cells that were treated with 200 μM H₂O₂ alone or co-treated with hESC-CM or dF-CM. As compared to the untreated control, the percent of apoptotic cells was much higher, and live cells significantly decreased with H₂O₂. Interestingly, the numbers of apoptotic cells robustly diminished and live cell numbers increased in cultures that were co-treated with H₂O₂ and hESC-CM, as compared to H₂O₂ alone or to H₂O₂ and dF-CM. Panel e) Quantification of Annexin V/7AAD Flow Cytometry experiments. The numbers of apoptotic cells robustly diminished and live cell numbers increased in cultures that were co-treated with H₂O₂ and hESC-CM, as compared to H₂O₂ alone or to H₂O₂ and dF-CM. *P<0.05, ***P<0.001.

FIG. 22, panels a-e, shows that human embryonic stem cell-derived conditioned medium (hESC-CM) protects motor neurons (MNs) from H₂O₂ cytotoxicity. Panel a) Differentiation of PSCs into MNs. The expression of stage specific markers for pluripotency (OCT4 and SOX2), motor neuron precursor (OLIG2), and MNs (TUJ1, BH9 and CHAT) by immunofluorescence; DAPI labels all nuclei. Scale bar, 100 μm. Panel b) Gene expression profiles of stage specific markers during differentiation into MNs by qRT-PCR that was performed in 3 replicates for each gene. ***P<0.001. After the motor-neuron identity and sufficient (above 90%) enrichment of these cultures were established, these human motor neurons were used in subsequent experiments. OLIG2 was detected at 14 days post differentiation. HB9, a specific marker for MNs, was observed at 30 days. Additionally, HB9 and CHAT gene expression were expressed at 30 days and as expected, these were lacking in the undifferentiated PSCs. In concert, the expression of pluripotency-related genes decreased dramatically upon the directed differentiation into the motor neuron lineage. Panel c) The viability of MNs was examined with different concentrations of H₂O₂ and with different concentrations of hESC-CM. Motor neuron viability decreased significantly (below 50%) by 200 μM H₂O₂ treatment and was not changed by up to 40% hESC-CM. Panel d) MTT assay for viability of MNs that were cultured with 200 μM H₂O₂ and indicated media and CMs. Panel e) Annexin V apoptotic index of MNs exposed to 200 μM H₂O₂ in indicated culture conditions (fold of the first replicate of Control). When motor neurons were treated with H₂O₂ there was markedly diminished viability and increased apoptosis as compared to the untreated controls. However, the resilience of MNs to H₂O₂ cytotoxicity was significantly increased, by the hESC-CM. This contrasted with dF-CM that failed to improve viability of these MNs in the presence of H₂O₂. Annexin V fluorescence was assayed by a SpectraMax iD3 microplate reader. Bar graphs show Means and SEM of three independent experiments and subsequent triplicate assays (MT, Flow cytometry, microplate reader of Annexin V). *P<0.05, **P<0.01 ***P<0.001.

FIG. 23 shows that antioxidant activity is not different between human embryonic stem cell-derived conditioned medium (hESC-CM) and differentiated fibroblast-derived conditioned medium (dF-CM). Each CM was collected after culturing the respective cells in OptiMEM for 24 hours. Antioxidant activity assay (BioAssay systems, DTAC-100 kit) was performed in triplicate wells for each hESC-CM, df-CM and the control medium, OptiMeM (optMEM), as recommended by the manufacturer. The antioxidant-reporting fluorescent signal intensity was measured by microplate reader (570 nm). The data are expressed as fold of the Mean of OptiMEM control, which was set at 1.

FIG. 24, panels a-b, illustrates differentiation of ALS iPSCs into motor neurons (ALS-MNs). Panel a) The expression of cell-fate markers for pluripotency (OCT4 and SOX2), motor neuron precursors (OLIG2), and MNs (TUJ1, BH9 and CHAT) by immunofluorescence; DAPI labels all nuclei. Scale bar, 100 μm. Panel b) qRT-PCR gene expression in 3 replicates for each gene assayed for the indicated cell-fate markers during differentiation of ALS-iPCSs into ALS-MNs ***P<0.001.

FIG. 25 illustrates profiling the time-point of the neuroprotective effect from iPSC-CM. The time course of viability (560/590 nm) and apoptosis (490/590 nm) of MNs that were pre-treated with iPSC-CM or post-treated (H₂O₂ first, then iPSC-CM).

FIG. 26, panels a-e, illustrates the comparative effects of autologous ALS-derived hiPSC-conditioned media (ALS-CM) on neurites and apoptosis of ALS-motor neurons (ALS-MNs) in ALS patient-derived cell lines. Panel a) Representative images of motor neurons with their neurites (CS07-ALS donor), (CS07-CM ALS donor iPSC conditioned medium) at 30 days and 60 days. Scale bar, 200 μm. Panel b) The neurite sizes of wild type and ALS-MNs were measured during cell differentiation (Upper). The net change of neurite size was calculated in these differentiating MNs (Below). Panel c) The neurite sizes were measured in ALS-MNs that were treated with autologous ALS-CM at different time points of their differentiation, d30, d35, d40, d45, and d50 (CS53, CS07). Panel d) Representative images of apoptotic Caspase 3 positive MNs at 30 days and 60 days of their directed differentiations of ALS iPSCs, CS07 line. Caspase 3 (green) was immunodetected with specific antibody, DAPI, blue stains all nuclei. Scale bar, 100 μm. Panel e) Quantification of apoptotic motor neuron precursors after H₂O₂ alone as compared to co-treatment with ALS iPSC-CM or dF-CM-Values are mean±SEM, *P<0.05, **P<0.01, N=triplicate assays for each condition.

FIG. 27 illustrates the effect of CMs on the weights in SOD1^{G93A} transgenic mice. The change of weights. There was no significant difference between ALS iPSC-CM group (N=16) and dF-CM group (N=6).

FIG. 28, panels a-b, illustrates the optimization of concentrations of heparin binding protein (HBP) and exosome. Panel a) MTT assay for viability of MNs that were cultured with different concentrations of HBP fraction of hESC-CM. Quadruplicate assays for each condition. Panel b) MTT assay for viability of MNs that were cultured with different concentrations of exosome fraction of hESC-CM. Triplicate assays for each condition. *P<0.05, Quadruplicates for each condition.

FIG. 29, panels a-c, shows the effect of epigenetic modifiers on the conditioned medium (CM). Panel a) Gene expression profiles of pluripotency markers after treatment of four epigenetic modifiers, Nanomycin A (NaA), LSD1 inhibitor (LSD1), 5 Azacytidine (5Aza), and GSK126. The expression of OCT4 was significantly decreased in the NaA, LSD1 and 5Aza groups compared to the untreated groups (WTC11 and CS07), but in the GSK126 group the decrease was not significant. NANOG expression significantly decreased in all groups treated with small molecules compared to the untreated groups. qRT-PCR was performed in 3 replicates for each gene. Panel b) The morphologies of induced pluripotent stem cells after the treatment of small molecules for 3 days. Scale bar, 200 μm. Panel c) The effect of epigenetic modifiers on the ability of CM to promote viability and inhibit apoptosis in ALS-MNPs. Bar graph represents Means and SDs of N=4 for each small molecule.

FIG. 30, panels a-b, illustrates differentially present proteins in each of the listed categories. Panel a) Violin plot of cohort distributions. Panel b) In the CC group, the top 10 most significantly up-regulated terms contained extracellular space, extracellular region, extracellular exosome, cell surface, blood microparticle, platelet alpha granule lumen, Golgi lumen, SMAD protein complex, membrane raft, and external side of plasma membrane. The top 10 most significantly up-regulated terms in the MF group comprised growth factor activity, cytokine activity, receptor binding, collagen binding, heparin binding, serine-type endopeptidase activity, tumor necrosis factor receptor binding, Ras guanyl-nucleotide exchange factor activity, protein homodimerization activity, and identical protein binding.

### DETAILED DESCRIPTION

In various embodiments, methods can compositions for protecting mammalian neurons from oxidative stress induced cell death are provided. In particular, it is demonstrated herein that proteins secreted by hESCs, iPSCs and importantly, derived ALS patient iPSCs, all protect human motoneurons (MN) from H₂O₂ oxidative stress induced cell death. Moreover, the secretomes of wild type and ALS patient iPSCs, are neuroprotective for ALS iPSC-derived MNs that have mutations in superoxide dismutase SOD1 (typical of familial ALS), and in the disease-relevant animal model of ALS—mice with the human SOD1 mutation that undergo progressive paralysis. In these models ALS iPSC derived secretome promotes viability of neurons, enhances the maintenance of axonal neurites, reduces harmful protein aggregation, delays the onset of disease in vivo, improves neuro-muscular function, brain and muscle health and innervation and delays the paralysis and morbidity in ALS mice. These neuroprotective properties are in the heparin-bound and soluble protein fractions (not in exosomes, lipid or sugar fractions of the secretome) and they strongly associated with pluripotency and are lost when PS cells are differentiated along the embryonic fibroblast lineage.

Comparative proteomics yielded 106 candidates that are present in the PSC-CM (hESC, iPSC, ALS iPSC) but are minimal in CM of the hESC-derived fibroblasts (see, e.g., Table 9). Analysis of these proteomics data supports the conclusion that PSC secretome has the capacity to protect neurons (etc. cells) from oxidative damage and increase cell viability, operating through multiple mechanisms. Summarily, this work comprehensively identifies a new therapeutic property of PSC secretome and suggest a rapid and autologous use of ALS patient iPSC secreted biologic to treat their disease.

Additionally, particular proteins were identified from the 106 candidates that individually, and in certain embodiments combinations thereof, have the capacity to inhibit apoptosis, attenuate oxidative stress, repair oxidative damage, and prevent protein aggregation and mislocalization. These results suggest that not just one, but multiple PSC-secreted biologic proteins can act in concert to protect and maintain neural cells in neurotoxic environments.

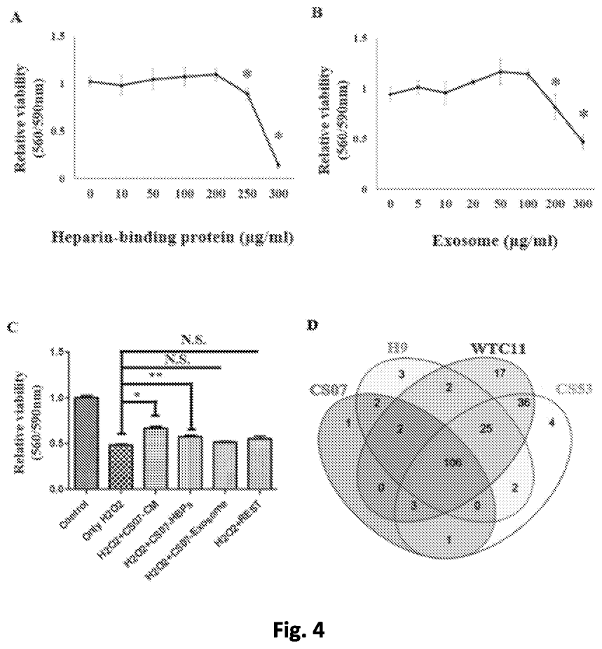

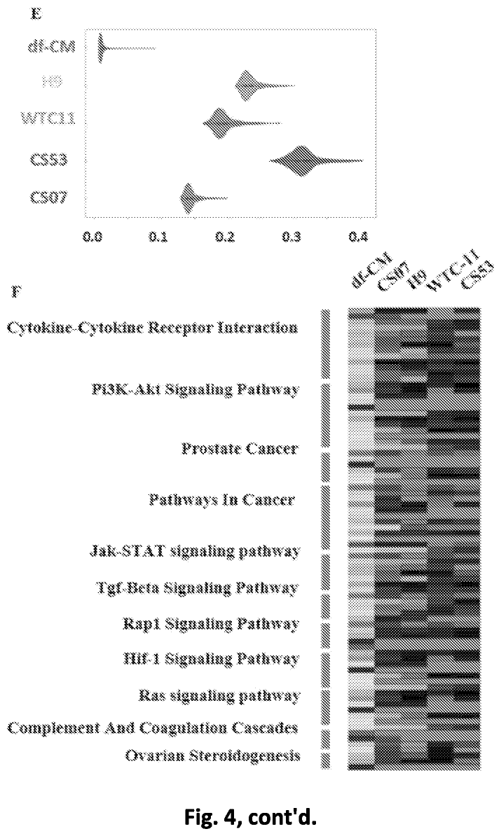

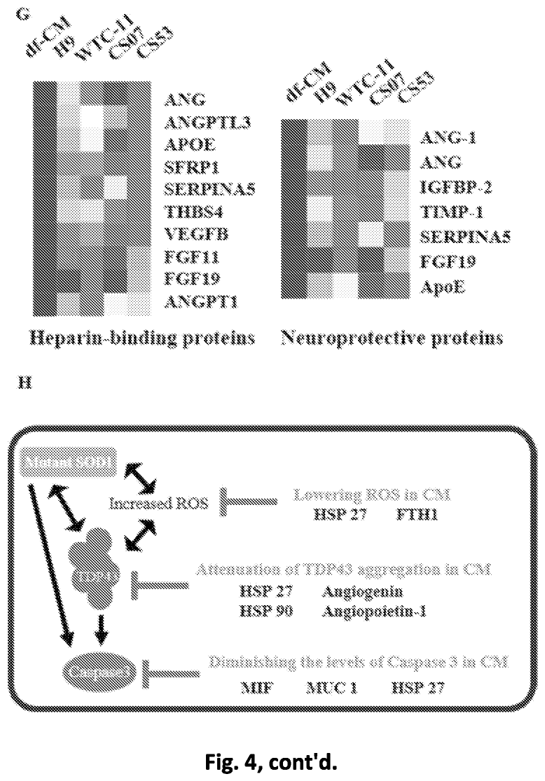

Accordingly it is recognized that compositions comprising at least 1 and in various embodiments, 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more of the proteins described herein (see, e.g., FIG. 4, panel G) are neuroprotective. Additionally, having identified the neuroprotective activity of the full-length proteins identified herein and combinations thereof, one of skill in the art would recognize that biologically active fragments of such proteins would be similarly neuroprotective. Similarly, one of skill in the art would recognize that biologically active analogs of such proteins can also function similarly to provide neuroprotective compositions.

It is believed that the neuroprotective compositions described herein are effective to, inter alia, reduce neuronal apoptosis as compared to an untreated control, and/or to diminish aggregation of TDP43 as compared to an untreated control, and/or to diminish neurite shrinkage as compared to an untreated control, and/or delay onset of paralysis, and/or delay morbidity, and/or delay denervation, and/or delay loss of muscle mass and function, as compared to untreated control. In fact, the neuroprotective compositions are believed to be generally neuroprotective, and specifically to be protective against oxidative stress and/or protein aggregation, and/or loss of innervation and synapses, and/or induced neural cell death and, accordingly find utility in the treatment and/or prophylaxis of numerous pathologies characterized by neurodegeneration.

Neuroprotection refers to the relative preservation of neuronal structure and/or function (see, e.g., Casson et al. (2012) Clin. Experiment. Ophthalmol. 40 (4): 350-357). In the case of an ongoing insult (e.g., a neurodegenerative insult) the relative preservation of neuronal integrity implies a reduction in the rate of neuronal loss over time (Id.). It is a widely explored treatment option for many central nervous system (CNS) disorders including neurodegenerative diseases, stroke, traumatic brain injury, spinal cord injury, and acute management of neurotoxin consumption (i.e. methamphetamine overdoses). Neuroprotection aims to prevent or slow disease progression and secondary injuries by halting or at least slowing the loss of neurons (see, e.g., Seidl & Potashkin (2011). Front Neurol. 2: 68). Despite differences in symptoms or injuries associated with CNS disorders, many of the mechanisms behind neurodegeneration are the same. Common mechanisms include increased levels in oxidative stress, mitochondrial dysfunction, excitotoxicity, inflammatory changes, iron accumulation, and protein aggregation (see, e.g., Seidl & Potashkin (2011). Front Neurol. 2: 68; Dunnett & Björklund (1999) Nature, 399(6738 Suppl): A32-39; Andersen (2004) Nat. Med. 10 Suppl (7): S18-25). Of these mechanisms, neuroprotective treatments often target oxidative stress and excitotoxicity—both of which are highly associated with CNS disorders. Not only can oxidative stress and excitotoxicity trigger neuron cell death but when combined they have synergistic effects that cause even more degradation than on their own (see, e.g., Zádori et al. (2012) J. Neurol. Sci. 322 (1-2): 187-191). Thus, limiting excitotoxicity and oxidative stress is a very important aspect of neuroprotection. Previous common neuroprotective treatments include, but are not limited to glutamate antagonists and antioxidants, which aim to limit excitotoxicity and oxidative stress respectively. These methods are of limited/temporary clinical usability, for example, anti-inflammatory and ROS reducing drugs (riluzole and edaravone⁴) slow down disease progression; later on, patients use respirators, speech facilitating devices, palliative care and ultimately, hospice care. In contrast to previous methods, in embodiment described herein the excitation of neurons (e.g., their function) is not inhibited and antioxidants are not used, but instead neurons' own anti-apoptotic properties and resilience to the oxidative damage are increased by the PSC secreted biologic.

IN view of the data presented therein is believe that the protein combinations and methods provided here are more generally effective to protect mammalian cells (e.g., neuronal and other cells) from oxidative stress, and/or mitochondrial dysfunction, and/or inflammatory gene expression, and/or protein aggregation, and/or toxin induced cell death.

It will be recognized that, in certain embodiments, the compositions described herein can be used alone, or in combination with other neuroprotective treatments such as the use of glutamate antagonists and/or antioxidants.

In certain embodiments the methods and neuroprotective compositions described herein find utility in the treatment or prophylaxis of a neurodegenerative pathology. In certain embodiments, the neurodegenerative pathology comprises a pathology selected from the group consisting of a motor neuron disease, Alzheimer's disease, Parkinson's disease, Huntington's disease, HIV-1 associated neurological degeneration, neurodegeneration associated with an ischemic event, neurodegeneration associated with traumatic brain injury (TBI), neurodegeneration associated with a spinal cord injury, drug-induced neurodegeneration, cancer-associated neurodegeneration, and glaucoma. In certain embodiments, the neurodegenerative pathology comprises a motor neuron disease (e.g., amyotrophic lateral sclerosis (ALS) or spinal muscle atrophy (SMA)). In certain embodiments, the neurodegenerative pathology comprises drug-associated (e.g., methamphetamine-associated) neurodegeneration. In certain embodiments, the neurodegenerative pathology comprises Alzheimer's disease. In certain embodiments, the neurodegenerative pathology comprises neurodegeneration associated with a stroke. In certain embodiments, the neurodegenerative pathology comprises glaucoma. In certain embodiments, the neurodegenerative pathology comprises any neurodegenerative pathology in which a reactive oxygen species (ROS) or other toxins, such as environmental toxins, are implicated.

In certain embodiments the methods and neuroprotective compositions described herein find utility in the treatment or prophylaxis of a mitochondrial disorders. Illustrative mitochondrial disorders include, but are not limited to Autosomal dominant optic atrophy, Alpers Disease or Syndrome, Barth Syndrome, Beta-oxidation defects, Carnitine Deficiency, Carnitine-Acyl-Carnitine Deficiency, Chronic Progressive External Ophthalmoplegia Syndrome (CPEO), Co-Enzyme Q10 Deficiency, Complex I Deficiency, Complex II Deficiency, Complex III Deficiency/COX Deficiency, Complex V Deficiency, CPT I Deficiency, CPT II Deficiency, Creatine Deficiency Syndrome, Kearns-Sayre Syndrome (KSS), Lactic Acidosis, Leukodystrophy (LBSL), LCHA Deficiency, Leber Hereditary Optic Neuropathy, Leigh Disease or syndrome, Long-Chain Acyl-CoA Dehydrongenase Deficiency (LCA Deficiency), Luft Disease, MAD/Glutaric Aciduria Type II, Medium-Chain Acyl-CoA Dehydrongenase Deficiency (MCAD), Mitochondrial Encephalomyopathy Lactic Acidosis and Stroke-like Episodes (MELAS), Mitochondrial DNA Depletion, Mitochondrial Encephalopathy, Mitochondrial Enoyl CoA Reductase Protein Associated Neurodegeneration (MEPAN), Mitochondrial Recessive Ataxia Syndrome (MIRAS), Myoclonic Epilepsy and Ragged-Red Fiber Disease (MERRF), Myoneurogastointestinal Disorder and Encephalopathy (MNGIE), Neuropathy, Ataxia, and Retinitis Pigmentosa (NARP), Neuropathy, Ataxia, and Retinitis Pigmentosa (NARP), Pearson Syndrome, Pyruvate Carboxylase Deficiency, Pyruvate Carboxylase Deficiency, Pyruvate Dehydrogenase Deficiency (PDC deficiency), Pyruvate Dehydrogenase Deviciency (PDC Deficiency), SANDO, SCHAD, Short-Chain Acyl-CoA Dehydrogenase Deficiency (SCAD), TK2/myopathic form, Very Long-Chain Acyl-CoA Dehydrogenase Deficiency (VLCAD), and the like.

### Active Agents and Modifications Thereof

### Active Agents.

As explained herein (see, e.g., Examples 1 and 2), a plurality of proteins that are secreted by induced pluripotent stem cells (iPSCs) derived from a mammal with amyotrophic lateral sclerosis (ALS) or any other disease where said proteins are secreted when said iPSC cells are pluripotent are neuroprotective against oxidative-stress induced neuronal cell death in a given disease. Additionally, it is believed that biologically active fragments of such proteins, and/or biologically active analogs of said proteins are similarly effective.

In particular, several heparin-binding proteins that were elevated in the PSC-CM cohorts as compared to the df-CMs, including angiogenin (ANG), Angiopoietin-1(ANG-1), angiopoietin like 3, apolipoprotein E (ApoE), secreted frizzled related protein 1, serpin family A member 5, thrombospondin 4, vascular endothelial growth factor B, fibroblast growth factor 11 (FGF11), and fibroblast growth factor 19 (FGF19). Proteins that do not bind to heparin, but are implicated in neuroprotection included ANG-1, ANG, IGFBP-2, TIMP-1, SERPINA5, FGF19, and ApoE.

Accordingly in various embodiments neuroprotective compositions are provided that comprise any one or more, or preferably any two or more of these proteins, either by themselves or combined with the iPSC secretome, or the unfractionated autologous iPSC secretome derived from patients, or heparin-bound and unbound protein fractions of such secretome; and such compositions are believed to be effective in the treatment and/or prophylaxis of pathologies characterized by acquired or genetic neurodegeneration.

In certain embodiments, the plurality of proteins comprises a protein selected from the group consisting of angiogenin (ANG), Angiopoietin-1(ANG-1), angiopoietin like 3, apolipoprotein E (ApoE), secreted frizzled related protein 1, serpin family A member 5, thrombospondin 4, vascular endothelial growth factor B, fibroblast growth factor 11 (FGF11), and fibroblast growth factor 19 (FGF19), or biologically active fragments or analogs thereof. In certain embodiments, the plurality of proteins comprises a protein selected from the group consisting of angiogenin (ANG), Angiopoietin-1(ANG-1), angiopoietin like 3, apolipoprotein E (ApoE), secreted frizzled related protein 1, serpin family A member 5, thrombospondin 4, vascular endothelial growth factor B, fibroblast growth factor 11 (FGF11), and fibroblast growth factor 19 (FGF19), or biologically active fragments or analogs thereof.

In certain embodiments, the plurality of proteins comprises a two or more proteins shown in Table 9 or biologically active fragments or analogs thereof.

In certain embodiments, the plurality of proteins comprises a protein selected from the group consisting of angiogenin

In certain embodiments, the plurality of proteins is effective to reduce cell apoptosis as compared to an untreated control and/or is effective to diminish aggregation of TDP43 as compared to an untreated control; and/or is effective to diminish neurite shrinkage as compared to an untreated control and/or avert loss of neuro-muscular function, and/or denervation, and/or morbidity, and/or loss of muscle mass, and/or loss of CNS neurons in vivo.

### Protecting Groups.

In various embodiments, the various proteins, protein fragments, or analogs thereof may be provided and are effective with no protecting groups. However, in certain embodiments they can bear one, two, three, four, or more protecting groups, e.g., to improve stability in formulations, to improve stability in administration, and/or to improve serum half-life. In various embodiments, the protecting groups can be coupled to the C- and/or N-terminus of the peptide(s) and/or to one or more internal residues comprising the peptide(s) (e.g., one or more R-groups on the constituent amino acids can be blocked). Thus, for example, in certain embodiments, any of the peptides described herein can bear, e.g., an acetyl group protecting the amino terminus and/or an amide group protecting the carboxyl terminus.

Without being bound by a particular theory, it is believed that addition of a protecting group, particularly to the carboxyl and in certain embodiments the amino terminus can improve the stability and efficacy of the protein(s).

A wide number of protecting groups are suitable for this purpose. Such groups include, but are not limited to acetyl, amide, and alkyl groups with acetyl and alkyl groups being particularly suitable for N-terminal protection and amide groups being particularly suitable for carboxyl terminal protection. In certain embodiments, the protecting groups include, but are not limited to alkyl chains as in fatty acids, propionyl, formyl, and others. Certain suitable carboxyl protecting groups include amides, esters, and ether-forming protecting groups. In one embodiment, an acetyl group is used to protect the amino terminus and an amide group is used to protect the carboxyl terminus. In certain embodiments blocking groups include alkyl groups of various lengths, e.g., groups having the formula: CH₃—(CH₂)ₙ—CO— where n ranges from about 1 to about 20, or from about 1 to about 16 or 18, or from about 3 to about 13, or from about 3 to about 10.

In certain embodiments, the acid group on the C-terminal can be blocked with an alcohol, aldehyde or ketone group and/or the N-terminal residue can have the natural amide group, or be blocked with an acyl, carboxylic acid, alcohol, aldehyde, or ketone group.

Other protecting groups include, but are not limited to Fmoc, t-butoxycarbonyl (t-BOC), 9-fluoreneacetyl group, 1-fluorenecarboxylic group, 9-florenecarboxylic group, 9-fluorenone-1-carboxylic group, benzyloxycarbonyl, xanthyl (Xan), trityl (Trt), 4-methyltrityl (Mtt), 4-methoxytrityl (Mmt), 4-methoxy-2,3,6-trimethyl-benzenesulphonyl (Mtr), mesitylene-2-sulphonyl (Mts), 4,4-dimethoxybenzhydryl (Mbh), tosyl (Tos), 2,2,5,7,8-pentamethyl chroman-6-sulphonyl (Pmc), 4-methylbenzyl (MeBzl), 4-methoxybenzyl (MeOBzl), benzyloxy (BzlO), benzyl (Bzl), benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl-Z), 2-bromobenzyloxycarbonyl (2-Br-Z), benzyloxymethyl (Bom), cyclohexyloxy (cHxO), t-butoxymethyl (Bum), t-butoxy (tBuO), t-Butyl (tBu), acetyl (Ac), and trifluoroacetyl (TFA).

Protecting/blocking groups are well known to those of skill as are methods of coupling such groups to the appropriate residue(s) comprising the peptides of this invention (see, e.g., Greene et al., (1991) Protective Groups in Organic Synthesis, 2nd ed., John Wiley & Sons, Inc. Somerset, N.J.). In illustrative embodiment, for example, acetylation is accomplished during the synthesis when the peptide is on the resin using acetic anhydride. Amide protection can be achieved by the selection of a proper resin for the synthesis. For example, a rink amide resin can be used. After the completion of the synthesis, the semipermanent protecting groups on acidic bifunctional amino acids such as Asp and Glu and basic amino acid Lys, hydroxyl of Tyr are all simultaneously removed. The peptides released from such a resin using acidic treatment comes out with the n-terminal protected as acetyl and the carboxyl protected as NH₂ and with the simultaneous removal of all of the other protecting groups.

### Increasing Serum Half-Life.

In certain embodiments, the proteins or protein fragments comprising the neuroprotective compositions described herein can be functionalized to increase serum half-life. Methods of modifying proteins to increase serum half-life are well known to those of skill in the art.

For example, pegylation is known to increase serum half-life and to reduce immunogenicity of proteins. Methods of pegylating peptides are well known to those of skill in the art (see, e.g., U.S. Pat. Nos. 7,256,258, 6,552,170, and 6,420,339, and the references cited therein).

Additionally, protein fusion technology is one of the most commonly used methods to extend the half-life of therapeutic proteins. For example, fusion with domain III of human serum albumin (3DHSA) can significantly improve serum half-life.

Another approach in the incorporation of tags into the protein that increase the interaction of the protein with fatty acids.

These approaches to increase serum half-life are illustrative and non-limiting. Using the aching provided herein, numerous other approaches will be available to one of skill in the arty.

Methods of Treatment and/or Prophylaxis

As noted above, in various embodiments the neuroprotective compositions described herein are effective in the treatment and/or prophylaxis of a pathology characterized by neurotoxicity, such as oxidative damage, and/or protein aggregation-induced neuronal cell death. In certain embodiments, the methods comprise administering the neuroprotective composition (e.g., the plurality of proteins and/or biologically active fragments of said proteins and/or analogs thereof, and/or unfractionated or fractionated patients' derived iPSC secretome) to a mammal in need thereof. In certain embodiments, the mammal in need thereof comprises a mammal diagnosed as having or as at risk for a neurodegenerative pathology (e.g., a motor neuron disease, Alzheimer's disease, Parkinson's disease, Huntington's disease, HIV-1 associated neurological degeneration, neurodegeneration associated with an ischemic event, neurodegeneration associated with traumatic brain injury (TBI), neurodegeneration associated with a spinal cord injury, environmental toxin and/or drug-induced neurodegeneration, glaucoma, and the like). In certain embodiments, the method provides protection of cortical neurons, hippocampal neurons, dopaminergic neurons. In certain embodiments, the method provides protection of spinal cord neurons. In certain embodiments, the method provides protection of motor neurons.

In certain embodiments, the method involves administration of the neuroprotective composition(s) to a mammal via a route selected from the group consisting of oral delivery, isophoretic delivery, transdermal delivery, parenteral delivery, aerosol administration, administration via inhalation, intravenous administration, and rectal administration. In certain embodiments, the methods involve administration to the brain or spinal cord of a mammal. In certain embodiments, the method involves intracerebral, ventricular or intrathecal delivery to a mammal. In certain embodiments, the methods involve intranasal delivery to a mammal. In certain embodiments, the methods involve delivery via an implant in a mammal.

In certain embodiments, the administration of the plurality of proteins and/or biologically active fragments thereof, and/or analogs thereof is effective to inhibit apoptosis, and/or to attenuate oxidative stress, and/or to repair oxidative damage, and/or prevent protein aggregation and mislocalization and/or prevent degradation of axonal projections, and/or prevent tissue denervation and promote innervation, when administered to the mammal.

In various embodiments, the neuroprotective compositions descry bed herein can be used for treatment of patients by means of a short-term administration, e.g., of 1, 2, 3 or more days, up to 1 or 2 weeks, in order to provide immediate neuroprotection. In another embodiment, the neuroprotective compositions can be used for treatment of patients by means of a long-term administration, e.g., multiple times a day, daily, weekly, monthly, etc. for 1 week or more, or for one month or more, or for 6 months or more, or for 12 months or more, for indefinitely, e.g., for the lifetime of the patient.

An effective amount of the neuroprotective compositions(s) in the methods provided herein may be determined empirically, for example, using animal models as provided herein. In vitro models are also useful for the assessment of effective amount.

### Formulations and Routes of Administration.

In certain embodiments, the neuroprotective compositions described herein are administered to a mammal in need thereof. In various embodiments the compositions can be administered to reduce, eliminate, or reverse neurodegeneration, particularly neurodegeneration associated with oxidative stress, protein aggregation, toxins, etc. that induce neuronal cell death. Such pathologies include, but are not limited to acquired and genetic motor neuron diseases (e.g., ALS, SMA), Alzheimer's disease, Parkinson's disease, Huntington's disease, HIV-1 associated neurological degeneration, neurodegeneration associated with an ischemic event, neurodegeneration associated with traumatic brain injury (TBI), neurodegeneration associated with a spinal cord injury, drug-induced or environmental toxin-induced neurodegeneration, glaucoma, and the like.

The proteins or protein fragments comprising the neuroprotective compositions can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of formulating such derivatives are known to those of skill in the art. For example, the disulfide salts of a number of delivery agents are described in PCT Publication WO 2000/059863 which is incorporated herein by reference. Similarly, acid salts of therapeutic peptides, peptoids (or other mimetics), and the like can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the PAC1 receptor agonists (MAXCAPs) described herein include halide salts, such as may be prepared using hydrochloric or hydrobromic acids. Conversely, preparation of basic salts of the PAC1 receptor agonists (MAXCAPs) described herein are prepared in a similar manner using a pharmaceutically acceptable base such as sodium hydroxide, potassium hydroxide, ammonium hydroxide, calcium hydroxide, trimethylamine, or the like. In certain embodiments basic salts include alkali metal salts, e.g., the sodium salt, and copper salts.

For the preparation of salt forms of basic drugs, the pKa of the counterion is preferably at least about 2 pH lower than the pKa of the drug. Similarly, for the preparation of salt forms of acidic drugs, the pKa of the counterion is preferably at least about 2 pH higher than the pKa of the drug. This permits the counterion to bring the solution's pH to a level lower than the pHₘₐₓ to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

In various embodiments preparation of esters typically involves functionalization of hydroxyl and/or carboxyl groups that are present within the molecular structure of the active agent. In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkylamine.

In various embodiments, the neuroprotective composition proteins or protein fragments described herein can be administered using any medically appropriate procedure, e.g., intravascular (intravenous, intraarterial, intracapillary) administration, injection into tissue, injection into cerebraspinal fluid, intracavity injection, and the like. In certain embodiments, parenteral, topical, oral, nasal (or otherwise inhaled), rectal, or local administration, such as by aerosol or transdermal administration for prophylactic and/or therapeutic treatment as described herein is contemplated. The compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

The proteins, protein fragments, and/or analogs described herein comprising the neuroprotective compositions can also be combined with a pharmaceutically acceptable carrier (excipient) to form a pharmacological composition. In certain embodiments, pharmaceutically acceptable carriers include those approved by a regulatory agency of the Federal or a state government or listed in the U.S. Pharmacopeia or other generally recognized pharmacopeia for use in/on animals, and more particularly in/on humans. A “carrier” refers to, for example, a diluent, adjuvant, excipient, auxiliary agent or vehicle with which an active agent of the present invention is administered.

Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., active peptide) and the resulting composition is compressed. Where necessary the compressed product is coated, e.g., known methods for masking the taste or for enteric dissolution or sustained release. Suitable coating materials include, but are not limited to ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain therapeutic applications, the neuroprotective compositions described herein are administered, to a patient in need thereof to prevent, reduce, slow, or reverse neurodegeneration in a pathology characterized by oxidative stress induced, protein aggregation induced, toxin-induced neuronal cell death. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the neuroprotective plurality of proteins, protein fragments, and/or analogs described herein to effectively treat (e.g., ameliorate one or more symptoms in) the patient.

The concentration of the neuroprotective plurality of proteins, protein fragments, and/or analogs described herein can vary widely, and will be selected primarily based on activity of the active ingredient(s), body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, or from about 3.5 mg/kg/day to about 7.2 mg/kg/day, or from about 7.2 mg/kg/day to about 11.0 mg/kg/day, or from about 11.0 mg/kg/day to about 15.0 mg/kg/day. In certain embodiments, dosages range from about 10 mg/kg/day to about 50 mg/kg/day. In certain embodiments, dosages range from about 20 mg to about 50 mg given orally once, twice, three times, or 4 times daily. It will be appreciated that such dosages may be varied to optimize a therapeutic and/or phophylactic regimen in a particular subject or group of subjects.

In certain embodiments, the neuroprotective plurality of proteins, protein fragments, and/or analogs described herein are administered to the oral cavity. This is readily accomplished by the use of lozenges, aerosol sprays, mouthwash, coated swabs, and the like.

In certain embodiments the neuroprotective plurality of proteins, protein fragments, and/or analogs described herein are administered systemically (e.g., orally, or as an injectable) in accordance with standard methods well known to those of skill in the art. In other preferred embodiments, the agents, can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

In certain embodiments, where local delivery is desired, administration may involve administering the composition to a desired target tissue, such as muscle, brain, spine, etc. For local delivery, the administration may be by injection or by placement of the composition in the desired tissue or organ by surgery, for example. In certain cases, an implant, such as a cannula implant, that acts to retain the active dose at the site of implantation may be used.

In some instances, hydrogel delivery may be employed, e.g., as described in Piantino et al. (2006) Exp. Neural. 201: 359-367; and Ma et al. (2007) Biomed. Mater. 2: 233-240. In some instances, systemic, intraperitoneal, intravascular or subcutaneous protocols are employed, e.g., as described in Pardridge (2008) Bioconjug. Chem. 19: 1327-1338. In some instances, nanoparticle mediated delivery protocols may be employed, e.g., as described in Tosi et al. (2008) Expert Opin. Drug Deliv. 5: 155-174. In some instances, intracerebral, ventricular or intrathecal delivery protocols may be employed, e.g., as described in Buchli & Schwab (2005) Ann. Med. 37: 556-367; and Shoichet et al. (2007) Prag Brain Res. 161: 385-392. In some instances, intranasal delivery protocols are employed, e.g., as described in Smith (2003) Drugs, 6: 1173-1177, and Vyas et al. (2006) Crit. Rev. Tuer. Drug. Carrier Syst. 23: 319-347.

In certain embodiments, intrathecal administration may be carried out through the use of an Ommaya reservoir, in accordance with known techniques (see, e.g., Balis et al. (1989) Am. J. Pediatr. Hematol. Oneal. 11: 74-76).

In some embodiments, the neuroprotective composition (or components thereof) may be formulated to cross the blood brain barrier (BBB). One strategy for drug delivery through the blood brain barrier (BBB) entails disruption of the BBB, either by osmotic means such as mannitol or leukotrienes, or biochemically by the use of vasoactive substances such as bradykinin. Other strategies for transportation across the BBB may entail the use of endogenous transport systems, including carrier-mediated transporters such as glucose and amino acid carriers, receptor-mediated transcytosis for insulin or transferrin, and active efflux transporters such as p-glycoprotein. Alternatively, drug delivery behind the BBB is by intrathecal delivery of therapeutics directly to the cranium, as through an Ommaya reservoir.

Methods of administration of the agent through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more

In certain embodiments proteins, and/or protein fragments, and/or analogs thereof described herein can be provided as a “concentrate”, e.g., in a storage container (e.g., in a premeasured volume) ready for dilution, or in a soluble capsule ready for addition to a volume of water, alcohol, hydrogen peroxide, or other diluent.

While the use of the neuroprotective compositions and/or components thereof described herein is described with respect to use in humans, they are also suitable for animal, e.g., veterinary use. Thus, certain preferred organisms include, but are not limited to humans, non-human primates, canines, equines, felines, rodents, porcines, ungulates, lagomorphs, and the like.

### Kits.

In various embodiments kits are provided for the use of the neuroprotective compositions described herein for the treatment and/or or prophylaxis of pathologies characterized by neurodegeneration. In various embodiments the kits comprise a container containing the neuroprotective compositions described herein and/or a container containing one or a plurality of the proteins, protein fragments, and/or analogs thereof described herein.

In addition, the kits optionally include labeling and/or instructional materials providing directions (e.g., protocols) for the use of the neuroprotective compositions described herein, e.g., alone or in with other agents that used for the treatment of neurodegenerative pathologies.

While the instructional materials in the various kits typically comprise written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Autologous ALS iPSC Secretome as a New Therapeutic for ALS

Amyotrophic lateral sclerosis (ALS) is a devastating, progressive neuro-degenerative disease with high morbidity and no cure. We previously reported positive effects of the human embryonic stem cell (hESC) secretome on skeletal muscle, neural precursor cells, and an ability to absorb/neutralize the toxic beta amyloid. Here, we found that proteins which are secreted by pluripotent stem cells (iPSCs), induced from male and female patients with ALS, rescue their motor neurons (MNs) from deterioration and death, and protect neurons in general from the deleterious consequences of reactive oxygen species (ROS) and protein aggregation. Moreover, male and female mice transgenic for the SOD1 mutation that associates with ALS and causes progressive neurodegeneration, have much improved neuro-muscular function, delayed disease onset and delayed morbidity when treated with the secretome of ALS patients' iPSCs. The state of pluripotency is required for the neuroprotective activity, it is mediated by proteins and it is contained in heparin-bound and soluble fractions of iPSC conditioned medium (CM), but not in the exosomes. In addition to anti-apoptotic properties, and improved neurite-maintenance, our results suggest a TDP43-related mechanism of the neuroprotection through iPSC secretome. Comparative proteomics yielded 106 candidates that are present in CM that manifest neuroprotection: of hESC, iPSC, ALS iPSC but are minimal in the negative control, e.g., CM of hESC-derived fibroblasts that lacks neuroprotective activity. The analysis of this comparative proteomics supports the conclusion of neuroprotection through multiple simultaneous mechanisms that operate via different proteins, which together confer resilience to cell stressors.

### Results

Human Embryonic Stem Cell Conditioned Medium Protects Cells from Oxidative Stress Induced Apoptosis.

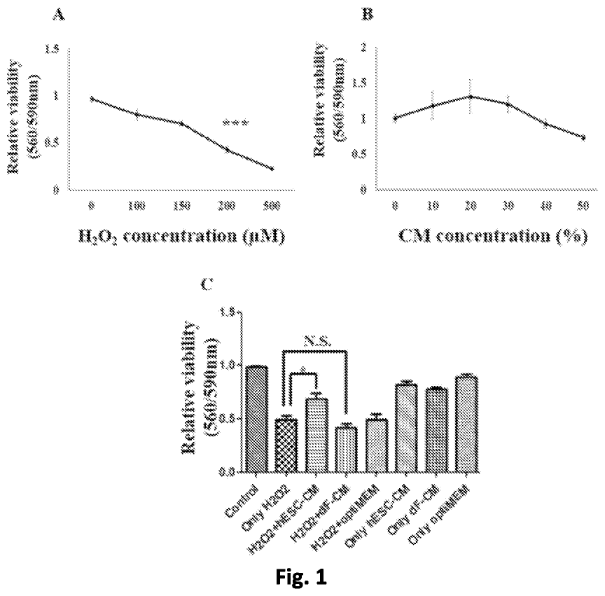

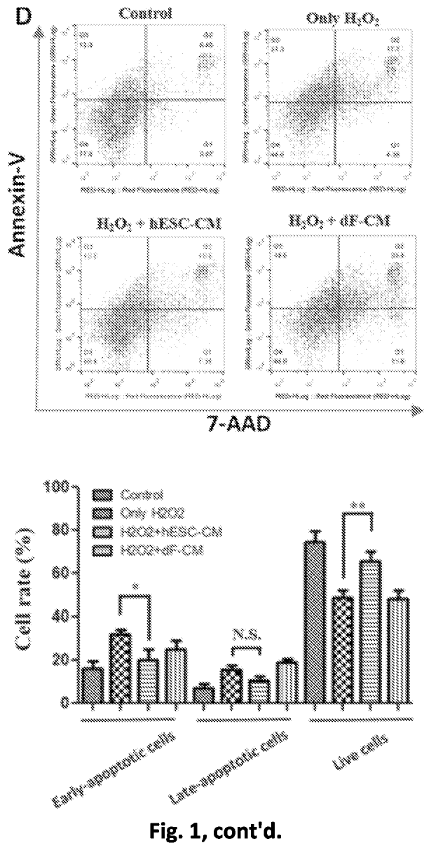

hESC conditioned medium was previously shown to enhance myogenic and neurogenic responses of tissue precursor cells (43, 44), but the effects on cell viability were less studied. We decided to explore a protective role of hESC conditioned medium (hESC-CM) when cells experience oxidative damage, which is physiologically and clinically relevant in a number of human diseases (56, 57). To set up this experimental system, we first performed dose response studies in IMR90 fibroblasts to varying concentrations of H₂O₂ and of hESC-CM using an MTT assay. Cell viability decreased significantly (below 50%) by 200 μM H₂O₂ treatment and was not changed by up to 40% hESC-CM (FIG. 1, panels A and B). An increase of MTT signal by 10-30% hESC-CM might indicate positive effects on fibroblast proliferation and/or on mitochondria.

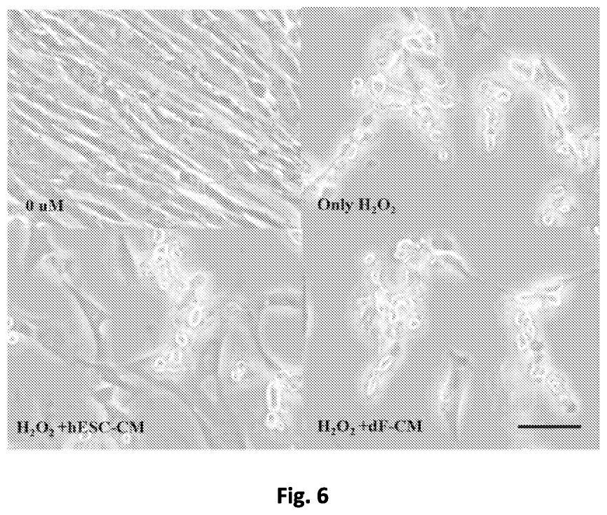

We then tested eight experimental cohorts, e.g., 200 μM H₂O₂ alone or in combination with 30% hESC-CM or CM from hESC-derived fibroblasts (dF-CM) (43) and controls for the various culture medium formulations (FIG. 1, panel C). Cell viability was noticeably lower in cultures treated with H₂O₂ and those co-treated with H₂O₂ plus the dF-CM than that in the H₂O₂ plus hESC-CM cultures (FIG. 1, panel C). The results of MTT assay and the conclusions on protection of human fibroblasts from H₂O₂-elicited cell death by hESC-CM were validated by cell morphology (FIG. 6).

To study the protective effects of hESC-CM in more detail, we performed Flow Cytometry on Annexin-V and 7-AAD. As compared to the untreated control, the percent of apoptotic cells was much higher, and live cells significantly decreased with H₂O₂, FIG. 1, panel D. Interestingly, the numbers of apoptotic cells robustly diminished and live cell numbers increased in cultures that were co-treated with H₂O₂ and hESC-CM, as compared to H₂O₂ alone or to H2O2 and dF-CM, (FIG. 1, panel D).

These data demonstrate that hESC-CM protects cells from the H₂O₂ caused apoptotic death. The antioxidant effects of hESC-CM were tested and not found to be different from the negative control dF-CM (FIG. 7). Thus, hESC-CM acts on cells and promotes their viability in the presence of H₂O₂, as it does not reduce the oxidative properties of H₂O₂ treated culture medium.

hESC-CM is Neuroprotective for Human Motor Neurons that are Exposed to H₂O₂.

Following the results with fibroblasts, we applied our experimental model to study effects of hESC-CM on MNs, where oxidative damage and apoptosis are implicated in the development of amyotrophic lateral sclerosis, ALS (58, 59). hESC-CM protected human cortical neurons from apoptosis induced by a/beta globulomers in culture, but this protection was, interestingly, through the effects of hESC-CM on de-toxifying the a/beta and not on the neurons (43).

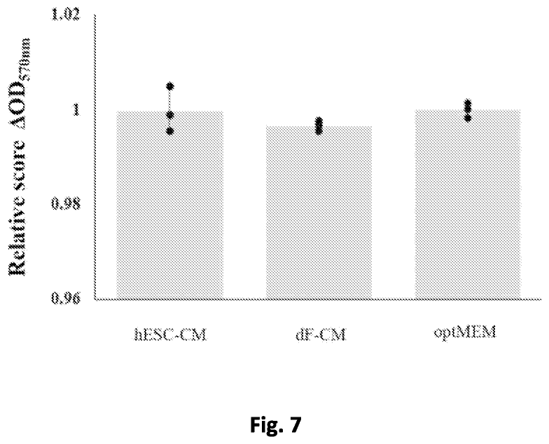

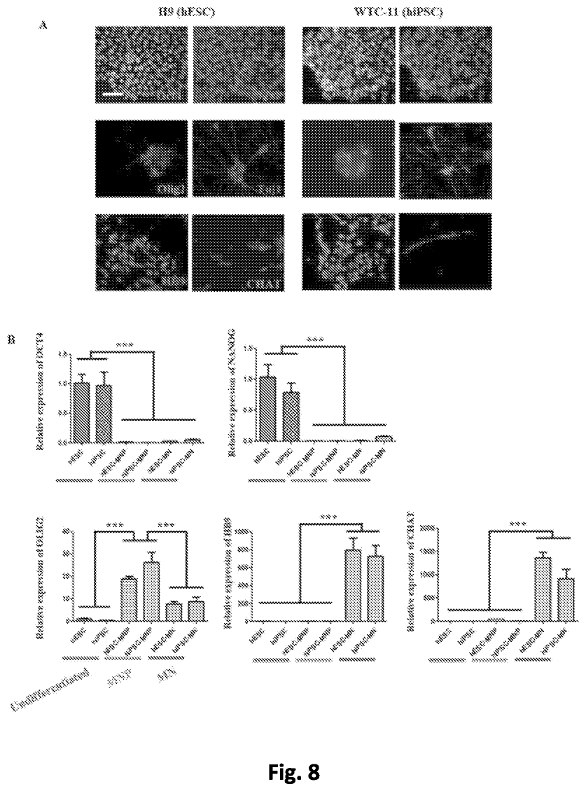

hESCs and human iPSCs were differentiated into human MNs, as described (60, 61). The success of differentiation was confirmed by immunofluorescence (FIG. 8A) and qRT-PCR (FIG. 8B) on cell-fate markers. The efficiency of MN differentiation was 92.52%, (Table 1).

TABLE 1
The efficiency of motor neuron differentiation
from normal pluripotent stem cells.

| H9 | | | WTC11 | | |
|---|---|---|---|---|---|
| | # of | | | # of | |
| # of | HB9 | | # of | HB9 | |
| DAPI | positive | % | DAPI | positive | % |
| 11270 | 10612 | 94.16 | 15642 | 14543 | 92.97 |
| 11130 | 10290 | 92.45 | 11818 | 10573 | 89.47 |
| 9654 | 8982 | 93.04 | 6492 | 6039 | 93.02 |
| | | 93.21 ± 0.86 | | | 91.82 ± 2.03 |

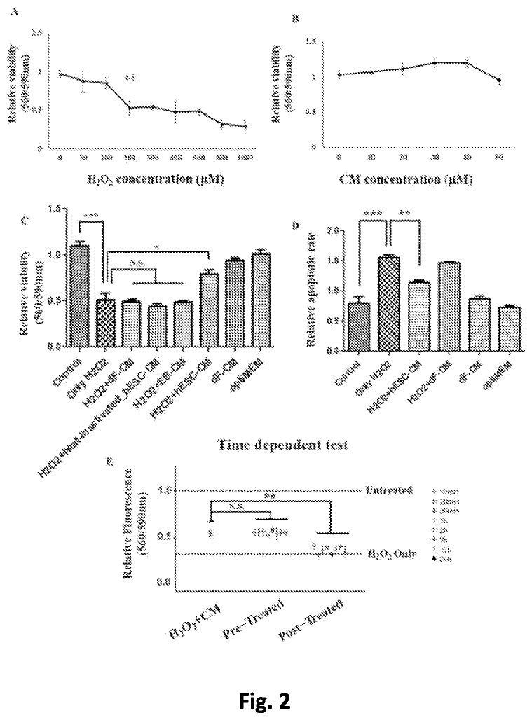

As above, we first determined a H₂O₂ and hESC-CM dose-responses in MNs and selected 200 μM H₂O₂ and 30% hESC-CM for further work (FIG. 2, panels A and B).

To examine the effects of hESC-CM on the viability of MNs that are exposed to H₂O₂, we performed the MTT and Annexin V assays. Motor neurons that were treated with H₂O₂ had markedly diminished viability and increased apoptosis as compared to the untreated controls (FIG. 2, panels C and D). Importantly, the resilience of MNs to H₂O₂ cytotoxicity was significantly increased, by the hESC-CM (FIG. 2, panels C and D). This was in contrast to dF-CM that failed to improve viability of these MNs in the presence of H₂O₂(FIG. 2, panels C and D).

We further explored the timing of this neuroprotective effect of hESC-CM on MNs by either pre-treating the cells with hESC-CM for variable time points before H₂O₂ (Pre-treatment) or alternatively, by treating the cells with H₂O₂ first for variable time points before adding the hESC-CM (Post-treatment). The viability assays demonstrated that hESC-CM is critically needed to be present at least, 10 minutes before H₂O₂ in order to protect the MNs from the cytotoxic effects, whereas the addition of hESC-CM after H₂O₂(10 minutes to 24 hours) does not confer this neuroprotection, FIG. 2, panel E.

These results demonstrate that hESC-CM protects human MNs from H₂O₂ cytotoxicity, while CM, which is produced by differentiated hESCs, does not have such neuroprotective properties, and suggests that whatever protective factor(s) are produced by hESCs must be present before the oxidative damage to exert their positive effects on human MNs.

### Autologous ALS IPSC-CM is Neuroprotective for Motor Neurons of ALS Patients

Encouraged by the neuroprotective effects of hESC-CM on human MNs, we tested the hypothesis that iPSC-CM will also be neuroprotective to, and will promote the viability of, ALS patient derived MNs. If true, this would suggest a novel completely autologous approach to treat ALS.

We studied two ALS patient derived iPSC lines (CS53-male and CS07-female) with A4V, e.g., the most common mutation in the SOD1 gene (Table 2).

TABLE 2
The list of amyotrophic lateral sclerosis cell lines.

| ALS-iPSC | Parent Cell | | | Age at Sample |
|---|---|---|---|---|
| Line Name | Type | Mutation | Sex | Collection |
| CS07iALS- | Fibroblast | SOD1 - A4V | Female | 40 |
| SOD1A4Vnxx | | (A5V) | | |
| CS53iALS- | Peripheral | SOD1 - A4V | Male | 35 |
| SOD1A4VNTnxx | blood | (A5V) | | |
| | mononuclear | | | |
| | cell | | | |

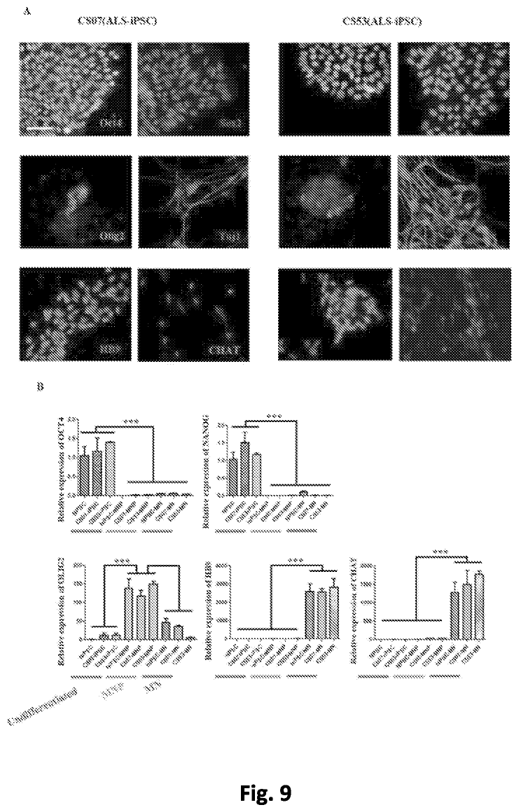

These ALS-iPSC lines were characterized for their markers of pluripotency and were differentiated into MN precursors (MNPs) and MNs, as confirmed by the immuno-detection of OLIG2, Tuj1, and HB9 (FIG. 9A) and by qPCR on OCT4 and NANOG, OLIG22, HB9, and CHAT (FIG. 9B). Interestingly, the wild type (WT) and ALS cells did not differ in the expression of pluripotency markers or morphology at iPSC stage, or in their MN markers, FIGS. 8 and 4. Additionally, there was no difference in the derivation efficiency of MN and ALS-MN (Tables 1 and 3).

TABLE 3
The efficiency of motor neuron differentiation from
ALS patient induced pluripotent stem cells.

| CS07 | | | CS53 | | |
|---|---|---|---|---|---|
| | # of | | | # of | |
| # of | double | | # of | double | |
| DAPI | positive | % | DAPI | positive | % |
| 18909 | 17273 | 91.35 | 20094 | 18149 | 90.32 |
| 6316 | 5776 | 91.45 | 11520 | 10430 | 90.54 |
| 5687 | 5103 | 89.73 | 11703 | 10256 | 87.64 |
| | | 90.84 ± 0.96 | | | 89.49 ± 1.61 |

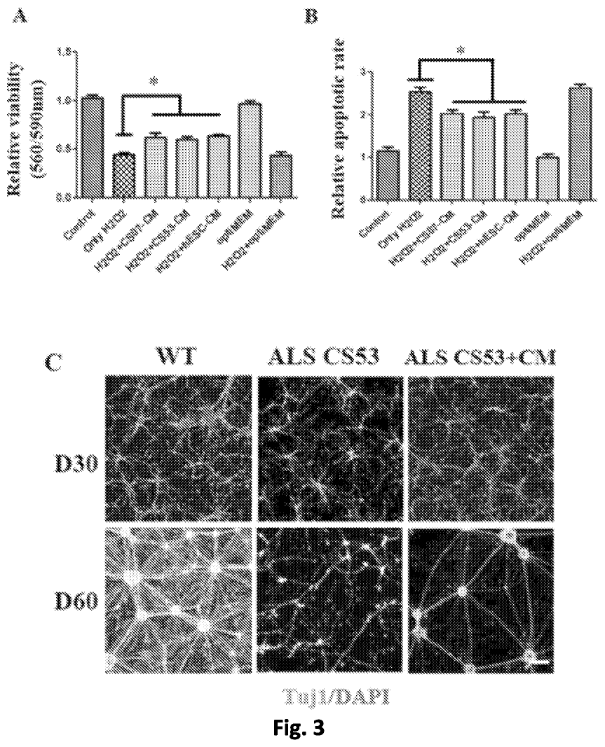

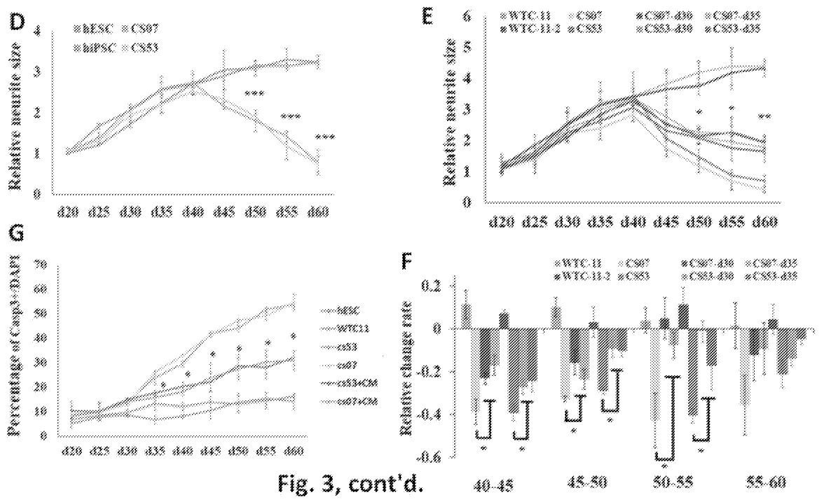

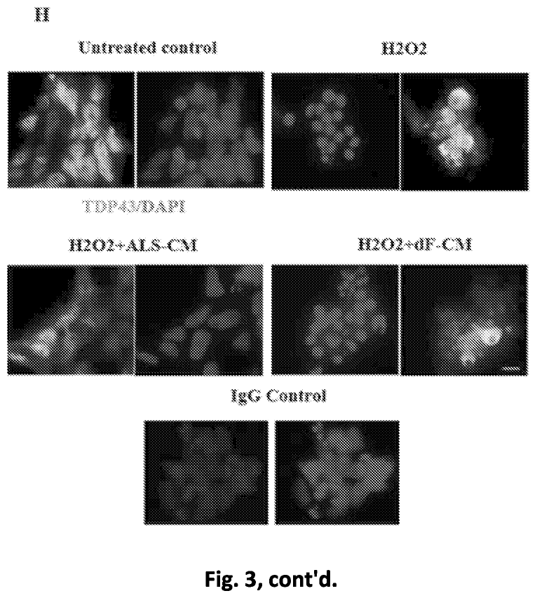

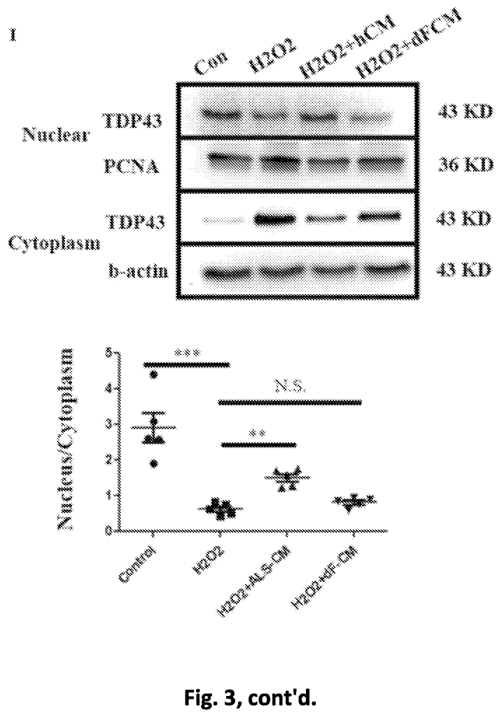

When exposed to H₂O₂, ALS-MNs had diminished viability and increased apoptosis, and interestingly all the tested PSC conditioned media, hESC-CM, WT iPSC-CM and most importantly the ALS patient derived iPSC-CM, had neuroprotective effects in this experiential set-up (FIG. 3, panels A and B).

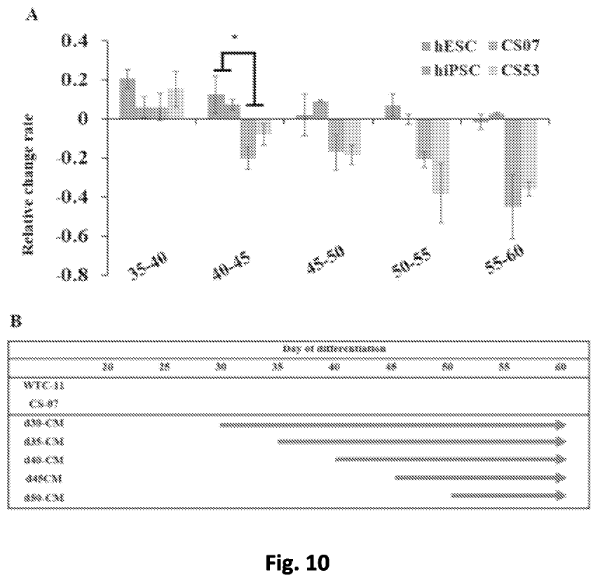

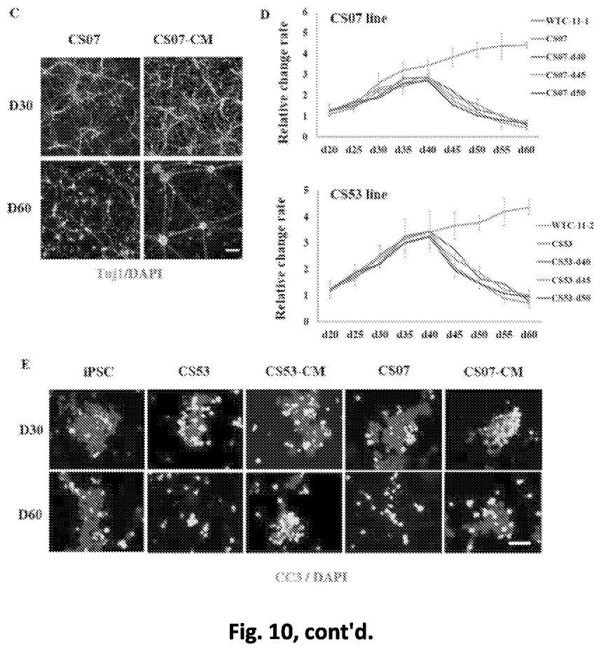

ALS-MNs are known to show a decrease in the size of their neurites as compared to wild type MNs (50), and thus we studied the effect of ALS-CM on this physiologically important parameter in two different primary ALS-MN lines (CS53 and CS07) that were treated with their autologous ALS iPSC-CMs. As shown in FIG. 3, panels C and D, neurite size increased gradually for 40 days of differentiation of iPSCs into MNs with no difference between the wild type and ALS cells. However, at 45 days of differentiation and all subsequent time points, the neurite size markedly shrunk in the ALS cohort as compared to the wild type cells (FIG. 3, panels C and D). Of note, neurite length fluctuation suggested that 40 to 45 days represent a key transition time-point of regulation of neurite outgrowth where shrinkage becomes prominent in ALS cells (FIG. 10, panels A and B).

The results of the 20-day studies (days 30 to 50 of directed differentiation) demonstrated that the maintenance of neurite sizes improved in the differentiating both lines of ALS-MNs that were treated with autologous ALS iPSC-CM starting at either day 30 or day 35 (FIG. 3, panel E). Moreover, the rate of neurite size degeneration was significantly reduced by ALS iPSC-CM at 30 days and at 35 days in both ALS-MN lines (FIG. 3, panel F). Additional data demonstrates that neurites were maintained better in the ALS iPSC-CM treated groups (FIG. 3, panel C and FIG. 10, panel C). However, when added at the later time point, e.g., at 35 days, ALS iPSC-CM was not able to rescue the neurite size of ALS-MNs (FIG. 10, panel D).

We also analyzed the relative numbers of apoptotic cells in these ALS-MNs (CS53 and CS07). There was a slight upward trend in the number of cleaved caspase3 (CC3) positive cells that was similar between the wild type and both ALS-MN lines up to day 30, but from day 35 onward the number of CC3 positive cells started to dramatically increase in ALS-MN cultures as compared to the wild type MN cultures (FIG. 3, panel G and FIG. 10, panel E). Notably, the rate of ALS-MNs apoptosis was significantly slowed by the autologous ALS iPSC-CM, for both cell lines (FIG. 3, panel G and FIG. 10, panel E).

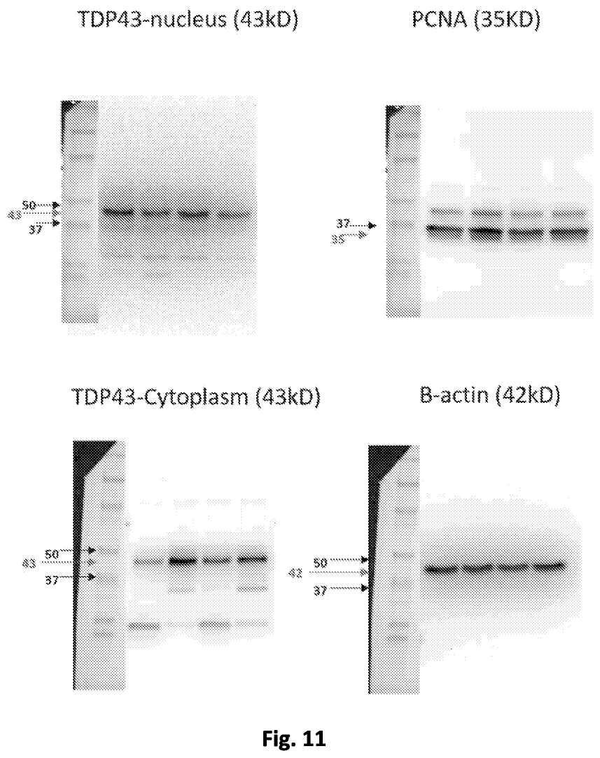

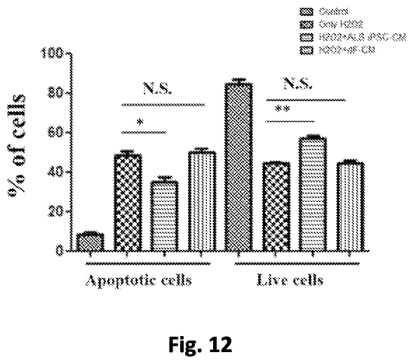

Finally, we examined aggregation of TDP43, considering that mutant SOD1 contributes to mislocalization of typically nuclear TDP43 to the cytoplasm (28). MNPs were derived from ALS-iPSCs CS07, as described above, and were treated with H₂O₂, which induced mislocalization of TDP 43 from nucleus to cytoplasm (FIG. 3, panels H and I). Importantly, such TDP-43 mislocalization was reduced by ALS iPCS-CM (but not by dF-CM) (FIG. 4, panels H and I). Namely, immunofluorescence assays suggested and Western Blotting on nuclear and cytoplasmic protein pools confirmed that the ratio of nuclear-to-cytoplasmic TDP43 became diminished by H₂O₂ and was restored to the control levels by the ALS iPSC-CM (FIG. 3, panels H and I, and FIG. 11). Immunofluorescence for TDP43 also indicated that aggregation of this protein in the cytoplasm (high intensity signal) was reduced by the ALS iPSC-CM (FIG. 3, panel H). Additionally, the apoptosis of MNPs was induced by H₂O₂ and was attenuated by the ALS iPSC-CM but not by dF-CM (FIG. 12).

Multiple Fractions and Proteins of PSC Secretome have Neuroprotective Capacity.

To start dissecting molecular mechanism(s) of the positive effects of PSC-CM on neural cells, we examined whether the neuroprotective activity is contained in the heparin-bound (HB) versus unbound fractions and exosome versus soluble fractions.

HB fraction became cytotoxic at 300 μg/ml (FIG. 4, panel A). Exosomes (purified from hESC CM by ultracentrifugation, (62), negatively affected cell viability at 200 μg/ml (FIG. 4, panel B). Based on these data, 200 μg/ml HBPs and 100 μg/ml exosome preparations were chosen for treating MNs.

There was an increase in viability and a decrease in apoptosis of MNs that were co-treated with H₂O₂ and HB fraction, as compared to H₂O₂ alone; however, the efficiency of that neuroprotection was less than half of that displayed by the unfractionated PSC-CM (FIG. 4, panel C). There was no improvement in viability or decrease in apoptosis of MNs that were co-treated with H₂O₂ and the exosome fraction (FIG. 4, panel C). The flow through CM, that did not contain exosomes or HB proteins, had a slight but not statistically significant trend of neuroprotection (FIG. 4, panel C). These data suggest that neuro-protective activity is contained in multiple fractions of PSC-CM: mostly in the soluble fractions (heparin bound and unbound).

Heat inactivation and Proteinase K treatments abrogated the positive effects of the iPSC secretome, demonstrating that neuroprotective activity is contained in the protein fraction (FIG. 4, panel B), and differentiation of PSCs into embryoid bodies similarly negated the neuroprotective activity, confirming that the state of pluripotency is required (FIG. 4, panel C).

To identify candidate neuroprotective proteins, we performed comparative 1000-factors proteomics arrays, comparing hESC-CM, WT iPSC-CM and ALS iPCS-CM (that all have neuroprotective activity), with each other, and with the dF-CMs that lack neuroprotective activity.

This screen identified 106 candidates (Venn diagram, FIG. 4, panel D), that were up-regulated in all PSC-CM groups, as compared to the dF-CM (>2-fold change with p<0.05). The violin plot showed that proteome patterns of hESC-CM, WT iPSC-CM and ALS iPSC-CM were significant different from those of dF-CM (FIG. 4, panel E). Heat Maps were constructed through grouping the proteins by their key functions using KEGG (FIG. 4, panel F). CMs and dF-CMs significantly differed in the levels of regulatory proteins that participate in canonical morphogenic signaling pathways, including CREB-cAMP, PI3K-Akt, Jak-STAT, BMP/TGF-β, and Ras, all of which were shown to be important for development, viability and/or maintenance of neurons (63).

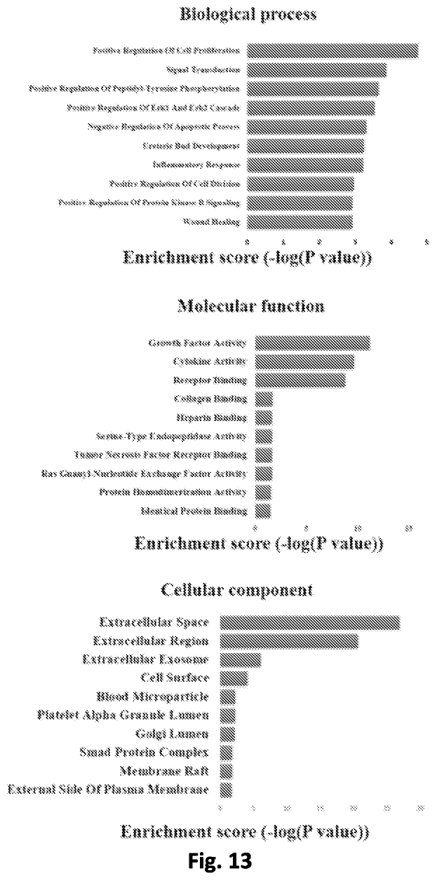

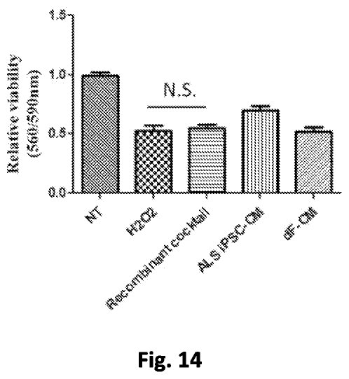

Additional protein characterizations as per the Biological process (BP), Molecular function (MF), and Cellular component (CC) by DAVID are shown in FIG. 13. In agreement with our functional data, the BP group had 170 GO terms, including positive regulation of cell proliferation, signal transduction, positive regulation of peptidyl-tyrosine phosphorylation, negative regulation of apoptosis, control of wound healing, and beta-amyloid clearance, the latter is highly consistent with our previously reported (43) neuroprotection from ectopic beta-amyloid (FIG. 13).

Interestingly, in concert with the above fractionation experiments and our previous findings on heparin-binding activity of pro-regenerative hESC-CM (43, 44) we identified several heparin-binding proteins that were elevated in the PSC-CM cohorts as compared to the dF-CMs, including angiogenin (ANG), Angiopoietin-1(ANG-1), angiopoietin like 3, apolipoprotein E (ApoE), secreted frizzled related protein 1, serpin family A member 5, thrombospondin 4, vascular endothelial growth factor B, fibroblast growth factor 11 (FGF11), and fibroblast growth factor 19 (FGF19), FIG. 4, panel G.

Among proteins that do not bind to heparin, but are implicated in neuroprotection (FIG. 4, panel G), TIMP1 showed protective effects against not only traumatic and ischemic brain injury but also HIV-1-induced neuronal apoptosis (64, 65). IGFBP-2 is involved in regulation of cell proliferation and it has an anti-apoptotic effect via modulation of caspase-3 (66, 67). Moreover, IGFBP-2 may have a role in neuroprotection from the hypoxic-ischemic injury and protects neurons from beta-amyloid-induced toxicity (68, 69). And most of above heparin-affinity factors promote cell proliferation and attenuate cell death (64, 70-73) in agreement with our observation in MNs and explaining previous reports anti-apoptotic effects of PSC-CM (44, 74, 75).

FIG. 4, panel H schematically outlines the mechanisms of PSC-CM exerted neuroprotection, illustrating the notion of simultaneous positive effects through activities of different PSC-CM proteins. For example, ANG has neuroprotective effect on hypoxia damaged MNs, ANG-1 inhibits neuronal apoptosis and enhances neurite outgrowth, ApoE reduces oxidative stress and contributes to beta-amyloid disposal (73, 76). Hsp27 has multiple functions as antioxidant (77), scavenger of TDP-43 aggregates (78), and inhibitor of procaspase-3 activation (79), HSP90 is related to the clearance cascade for TDP-43 (80). AKT pathway, as well as FTH1(80), macrophage migration inhibitory factor (MIF) (81), and MUC1 (82) all inhibit apoptotic cell death via separate mechanisms.

### Neuroprotection, Improved Neuro-Muscular Function and Delayed Morbidity of Male and Female ALS Mice Bearing the Human Disease SOD1^{G93A}.

Transgenic mice engineered to carry the mutant human SOD1 (SOD1^{G93A}) gene are used extensively to study human diseases (84, 112, 113). These mice display progressive degeneration of MNs and the phenotypes of ALS and thus, we decided to use this model for assessing the effects of ALS iPSC secretome in vivo. The ALS like symptoms are exhibited by the SOD1^{G93A} mice through the entire progression of the disease.

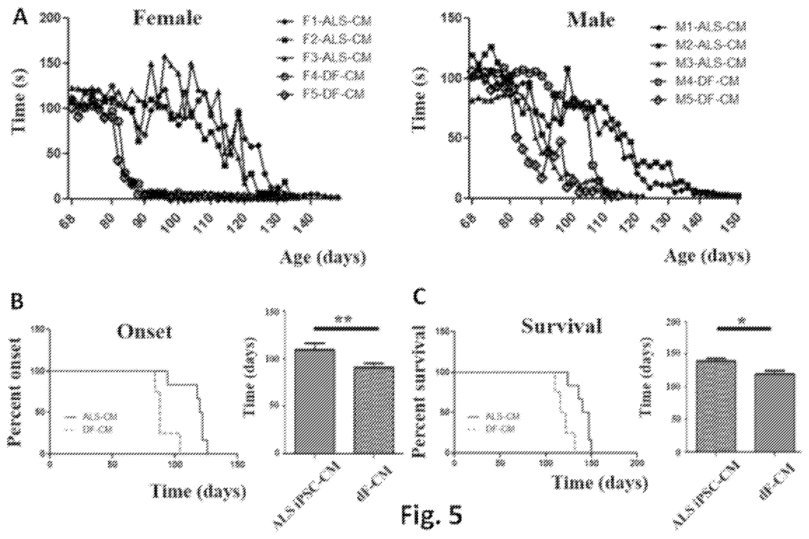

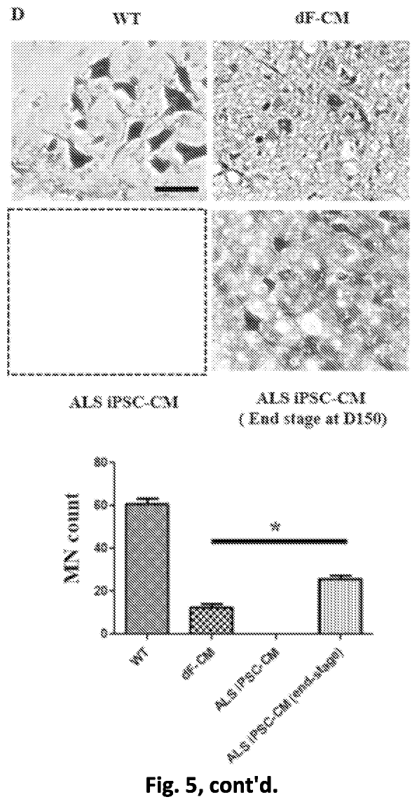

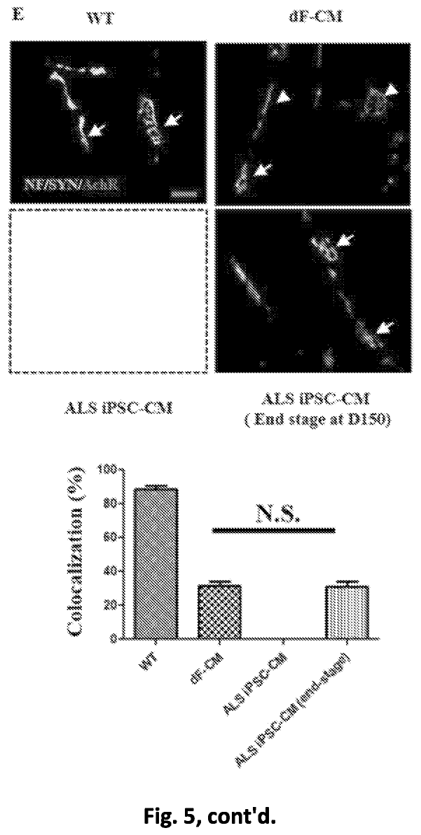

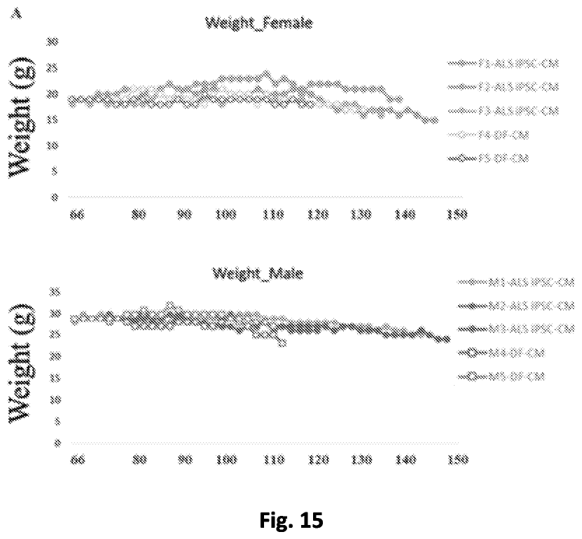

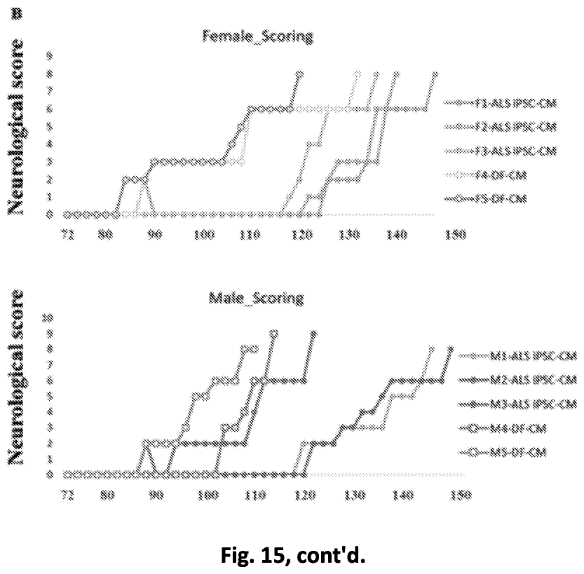

Until the endpoint, there was no significant difference in animal weights between ALS iPSC-CM treated and the negative control, dF-CM treated, groups (FIGS. 15A and B), which is consistent with (83). The control df-CM treated mice rapidly deteriorated in their neuro-muscular function, which we measured through the neurological scoring (84) and four-limb hanging test. Remarkably, CM of ALS patients' iPSCs rescued the agility, coordination and overall muscle function of the SOD1 mutant mice and delayed the onset of pathological symptoms. The decline reached under 50 seconds at 88 days in the dF-CM treated group (FIG. 5, panel A), whereas the hanging time in the ALS iPSC-CM group went below 50 seconds at 112 days. Significant differences between ALS iPSC-CM group and dF-CM group were detected at 88 days (96.83±5.74 vs. 41.5±17.78, P<0.05) and 112 days (68.33±13.32 vs. 23.0±12.46, P<0.05). Onset: (117.0±4.72 days vs. 91.00±4.43 days, P<0.05), consistent with their neurological scoring (FIG. 5, panel B and FIG. 15, panels C and D).

Importantly, and in agreement with the above neuro-muscular improvements the survival time was significantly prolonged in the ALS iPSC-CM group as compared to the dF-CM group (140.7±3.94 days vs. 120±4.69 days, P<0.05) (FIG. 5, panel C).

To investigate the effect of hiPSC-CM on the lumbar spinal cord and neuromuscular junction, these structures were analyzed postmortem in 120 days old mice. Compared to the C57.B6 wild type animals, the total number of cresyl violet stained MNs in the ventral spinal cord was decreased in the SOD1^{G93A} mice that were treated with control dF-CM (60.83±2.44 vs. 12.5±1.70, P<0.0001) (FIG. 5, panel D). In addition, many cresyl violet stained MNs showed shrunken morphology with no neurites in the SOD1^{G93A} dF-CM group as compared to the C57.B6 (FIG. 5, panel D).

Interestingly, the number of MNs was significantly increased in the SOD1^{G93A} ALS iPSC-CM treated mice as compared to the dF-CM group (? vs. 12.5±1.70, P<?-Currently in progress) and was greater than in the negative control even at the end-stage (at 150 days) group (25.83±1.537 vs. 12.5±1.70, P<0.05). These results demonstrate that the treatment of ALS model mice with ALS iPSC-CM may prevent MNs in the spinal cord from deterioration.

Next, we investigated the changes in the muscles of the transgenic mice after administration of CMs. First, the weights of Gastrocnemius (GA) and Tibialis anterior (TA) were measured and normalized by animal body weight. As expected, there was significant decrease in the average weights of GA and TA in the ALS iPSC-CM and the dF-CM compared to the C57.B6 group (FIG. 15, panels A and B). Interestingly, the weights of GA and TA were significantly increased in the ALS iPSC-CM group, as compared to the dF-CM group.

NMJ defects contribute to the changes in the muscle mass in general and might be involved in ALS (85-87), thus, we investigated the NMJ health and muscle innervation, using specific markers: acetylcholine receptor (AchR), neurofilament and synaptophysin. Our result show that the number of NMJs was significantly reduced in the ALS-CM and dF-CM groups, as compared to the C57.B6 group at day 120 (88.67±1.97 vs. 31.75±2.09, P<0.001, FIG. 5, panel E), while the ALS-CM group showed significantly more intact NMJs than the dF-CM group in the SOD1^{G93A} mice (? vs. 31.75±2.09, P<?—Currently in progress). At end-stage, there was no difference between dF-CM and the ALS iPSC-CM group (FIG. 5, panel E). These findings suggest that the treatment of ALS iPSC-CM may protect muscle innervation during the disease progression, thus resulting in delayed muscle atrophy in SOD1^{G93A} mice.

Summarily, these data demonstrate that ALS iPSC-CM significantly delays the onset of the functional decline in the SOD1^{G93A} mice, extends the life-span and protects MNs, NMJs and muscle mass in this animal model of human progressive neurodegenerative disease.

### DISCUSSION

Our results demonstrate that proteins secreted by hESCs, hiPSCs and importantly, ALS patient iPSCs, all protect human MNs from environmental and genetic oxidative stress. Specifically, the secretome of ALS patient iPSCs attenuates key pathological phenotypes of ALS not only in MNs of ALS patients, but also in vivo in the ALS mouse model. These conclusions are supported by the studies with male and female ALS iPSC-CM, MNs and SOD1 mutant mice.

The use of iPSCs for derivation and transplantation of neurons and neuronal precursor cells, while promising, has the problem of incomplete differentiation, and thus the danger of transplanting cancer-causing cells. Cell based therapies also have difficulties scaling up for enough cells, e.g., multiple transplantations are needed, and iPSC-derived tissues are difficult to mature. Our approach avoids these problems by using secreted proteome of iPS cells. CM is relatively easy to scale up, preserve long term and transport.

Several clinical trials suggest positive effects of mesenchymal and adipose stem cells' secretomes (31, 38, 116, 117), but interestingly, in our experimental system, pluripotency is required for the positive effects of CM (iPSC, hESC, ALS iPSC) and even partial differentiation into EBs negates neuroprotection. This conclusion fits well with the notion that pluripotent stem cells maintain low levels of oxidative stress via oxidation-reduction (redox) signaling, as compared to their differentiated progeny (100-103). We show that not just iPSCs themselves are protected, but that the anti-apoptotic and neuroprotective properties are conferred by the iPSC' secretome onto human MNs.

Reprogramming of disease-specific differentiated cells into iPSCs often results in normal pluripotency, even when there are disease-associated mutations, and in our work, ALS-iPSCs were typical pluripotent stem cells, based on their markers and the efficiency of MN differentiation (108-110). Interestingly, the activity of PSC-CM was contained in soluble fraction and not in the exosomes, which have known effects on cell growth, and viability in other experimental systems (111).

Transgenic mice with the human SOD1^{G93A} mutation are widely used to study human ALS (84, 112, 113) (115). Our results are the first to demonstrate the positive effects of ALS patients' derived iPSC CM in this model, with moreover, robust multi-parametric improvements, e.g., delay in the onset of paralysis and morbidity, enhanced neuro-muscular functionality, survival of spinal cord MNs and maintenance of NMJs and muscle mass. The administration of CM started at 68 days before the onset of symptoms, because our results consistently demonstrate that there is a time window during which the PSC secretome can rescue MNs from apoptosis and neurite shrinkage caused by oxidative damage, suggesting that early interventions are important. Future work will explore the minimum onset-interval of the ALS iPSC-CM treatment that might be optimally therapeutic.

One important effect of PSC-CM that we found was attenuation of TDP43 aggregation and diminishing inappropriate localization of this protein to the cytoplasm. TDP-43 plays a key role in pathogenesis of ALS and other neurodegenerative diseases (88), where mislocalization causes p53-based neuronal apoptosis and increases Cas3 activation (15-17, 28).

Our comprehensive proteomics identified specific candidates with capacity to inhibit apoptosis, attenuate oxidative stress, repair oxidative damage, and prevent protein aggregation and mislocalization. These results suggest that not just one, but multiple PSC-CM proteins can act in concert to protect and maintain neural cells in neurotoxic environments. Dissecting the roles of some of these proteins, attenuators of TGF-beta pathway by the PSC secretome might counter pro-inflammatory and pro-fibrotic properties of this pathway that becomes excessive in degenerative diseases and with oxidative damage (in general and in SOD1^{G93A} mutant mice), (89, 90, 99, 91-98). In agreement with observed support for neurite size, GDF11 is produced by de-novo differentiated MNs and promotes cell cycle exit and MN formation (91). Comprehensive future work will profile all PSC candidates for their cross-talks and for causality of neuroprotection.

Neuronal cell death is causal in many neurodegenerative diseases, including age-related loss of memory and dementias (such as Alzheimer's Disease), Parkinson's Disease, traumatic brain injury, strokes, spinal cord injury, and motor function degenerative diseases, such as ALS and spinal muscle atrophy (SMA). These diseases are characterized by neuroinflammation and oxidative cell damage, many involve perturbed proteostasis and all are devastating and without a cure. Thus, the iPSC secretome has clinical capacity for a broad class of neurodegenerative pathologies, and diseases of excessive cell apoptosis (50, 104-107).

### Materials and Methods

### Animals

All animal experimental procedures were performed in accordance with the Guide for Care and Use of Laboratory Animals of the National Institutes of Health, and approved by the Office of Laboratory Animal Care (OLAC), UC Berkeley.

Transgenic mice SOD1^{G93A} (B6SJL-Tg(SOD1*G93A)1Gur/J) were purchased from Jackson Laboratory (No. 002726; Bar Harbor, USA). All mice were randomly assigned to an ALS iPSC-CM group or a dF-CM group.

### Culture of Human Pluripotent Stem Cells and Collection of the Conditioned Medium

Human embryonic stem cells (hESCs, H9, WiCell Institute) and human induced pluripotent stem cells (hiPSC, WTC-11) were obtained through the UC Berkeley Cell Culture Facility. The SOD1-mutated familial ALS-iPSCs, CS07 and CS53, were obtained from Cedars-Sinai medical center (Los Angeles, California) (Table 3). All human pluripotent stem cells (hPSCs) were maintained on plates coated with Vitronectin (Life technologies) and cultured in Essential-8 medium (Life technologies) at 37° C. in a 5% C02 atmosphere. All hPSCs were passaged every 7 days by 0.5 mM EDTA (Life technologies). For the collection of conditioned medium, hPSCs were washed twice with Opti-MEM (ThermoFisher, Carlsbad, CA) and cultured in Opti-MEM for 24 hours and the conditioned medium then collected as hESC-CM, hiPSC-CM, and ALS-CM as published (43). The collected CMs were centrifuged at 10000×g for 30 minutes at 4° C. and stored at −80° C. before use. For the CM from differentiated cells, fibroblast-like cells were derived from hESCs and hiPSCs. hPSCs were harvested by 0.5 mM EDTA, transferred to 100-mm Petri dishes and cultured with Essential 6 (Life technologies) at 37° C. in a 5% CO₂ atmosphere for 5 days to generate embryoid bodies (EBs). Then, EBs were transferred to a 100 mm tissue culture dish (Falcon) and cultured in Dulbecco's Modified Eagle's Medium and Ham's F-12 Nutrient Mixture (DMEM/F12) containing 10% fetal calf serum (FCS), 1% (v/v) penicillin-streptomycin. When confluent, hPSC-derived fibroblast-like cells were passaged using 0.05% trypsin. After 2 passages, the collection of CM from the hESC-derived fibroblasts was conducted with the same protocol as the hPSCs.

### Culture of Human Fibroblast Cell Line

Human lung fibroblast cells, IMR-90 (CCL-186, ATCC), were maintained in DMEM (Dulbecco's Modified Eagle Medium) supplemented with 10% fetal calf serum (FCS, Hyclone) containing 1% penicillin-streptomycin (Invitrogen) and maintained in a humid atmosphere at 37° C. containing 5% CO₂. When cells were grown to 70% confluence, they were subcultured at 1/5 dilution for later passaging.

### Motor Neuron Precursor (MNP) and Motor Neuron (MN) Differentiation

The process of MNP and MN differentiation was as published (61). Briefly, hPSCs were detached by 0.5 mM EDTA and split 1:10 on Matrigel-coated plates (1:50 dilution). After one day, the Essential 8 was changed to neural inducing medium containing neural basal medium (NBM) plus 3 μM CHIR99021 (Tocris), 2 μM LDN-193189 (Tocris) and 3 μM SB431542 (Stemgent). The NBM is composed of DMEM/F12, Neurobasal medium (FisherScientific) at 1:1, N2, B27, 0.1 mM ascorbic acid (Millipore-Sigma), 1× Glutamax and 1× penicillin/streptomycin (all from Invitrogen). The inducing medium was maintained for 5 days and changed every other day. On day 5, the cultured cells were split at 1:6 with Accutase and cultured in NBM containing 3 μM CHIR99021, 2 μM SB431542, 0.1 μM retinoic acid (RA, Stemgent) and 0.5 μM purmorphamine (Pur, Stemgent). After 7 days, MNPs were generated and expanded with MNP medium, which consisted of NBM supplemented with 3 μM CHIR99021, 2 μM SB431542, 0.1 μM RA, 0.5 μM Pur and 0.5 mM valproic acid (VPA, Stemgent). MNPs were passaged every 7 days by exposure to Accutase and cryopreserved in liquid nitrogen in DMEM/F12, 10% Knockout serum replacement and 10% DMSO. To induce MN differentiation, MNPs were dissociated with Accutase and cultured in suspension in the above NBM with 1 μM RA and 0.1 μM Pur. The medium was changed every other day. After 7 days, MNPs differentiated into MNs. The MNs were then dissociated with Accutase into single cells and plated on PLO/laminin-coated plates. The MNs were cultured into mature neurons in NBM plus 1 μM RA, 0.1 μM Compound E (Calbiochem), 10 ng/ml Insulin-like growth factor 1, 10 ng/ml Glial cell-derived neurotrophic factor (ThermoFisher), and 10 ng/ml Brain-derived neurotrophic factor (ThermoFisher).

Isolation of Heparin-Binding Proteins (HBPs) from Conditioned Medium

The HBPs of hESC-CM were prepared as published (43). Briefly, Heparin-Agarose Type I Beads (H 6508, Sigma Aldrich) were washed with molecular grade water and incubated with CM from the various cells for 2 hours with agitation at 4° C., then samples were centrifuged at 10,000 g for 20 minutes. The collected heparin beads were washed two times for 10 minutes at 4° C. in washing buffer (1 ml phosphate-buffered saline (PBS)+0.05% Tween-20). The HBPs were eluted twice for 15 minutes at 4° C. in 400 μl of elution buffer (0.01M Tris-HCl pH 7.5+1.5M NaCl+0.1% BSA). The collected proteins were re-equilibrated to the cell culture medium by dialysis for 2 hours at 4° C. in 500 ml McCoy's 5A Medium (Gibco) followed by overnight dialysis at 4° C. in 200 ml Opti-MEM (Gibco). The eluted heparin bound proteins were diluted to 800p with Opti-MEM and stored at −80° C. before use.

Isolation of Exosomes from Conditioned Medium

For the isolation of exosomes, 100 ml samples of hESC-CM were centrifuged at 2,000 g for 10 minutes to remove cells and debris, followed by centrifugation at 10,000 g for 30 minutes at 4° C. to remove microvesicles (62). The supernatant was filtered through 0.22 μm filters (Millipore-Sigma) and then the filtered CM was sedimented by ultra-centrifugation at 120,000 g for 90 minutes in an i70 rotor (Beckman Coulter, Brea, CA, US). The pellet was washed once with PBS and re-sedimented at 120,000 g, 4° C. for 90 minutes. Finally, the pellet was resuspended in 200 μl PBS and stored at −80 C for further use.

### Cell Viability Assay

After H₂O₂ and/or CM or CM-derived factors treatment, the number of viable cells was determined by a resorufin based assay using the commercially available CellTiter-Blue® cell viability assay (G8080, Promega), as per the manufacturer's protocol. MNs were cultured in a 96-well plate at a cell density of 2×10⁴ and treated with H₂O₂ and/or CMs for 24 hours before performing the viability assay. A SpectraMax iD3 Multi-Mode Microplate Reader (Molecular Devices) was used for measuring fluorescence intensity (560/590 nm).

### Antioxidant Assay

The antioxidant assay was measured according to the manufacturer's instructions (BioAssay systems, DTAC-100). Briefly, each sample (20 μL) was mixed with kit reagent (100 μL). Then the mixed sample was incubated 10 min at room temperature. The color intensity at 570 nm is measured by a SpectraMax iD3 Multi-Mode Microplate Reader. The values ranging from 0-1000 μM Trolox were used as a reference.

### Real-Dine Polymerase Chain Reaction (PCR)

Total RNA was extracted using the RNeasy mini kit (Qiagen), and the SuperScript III First-Strand Synthesis System (Invitrogen) was used to synthesize cDNA according to the manufacturer's instructions. Real-time PCR was performed on a Bio-Rad iQ5 real-time PCR machine. The primers used for PCR are listed in Table 4.

TABLE 4
Primer sequences for Real
Time Polymerase Chain Reaction

| | Gene | | SEQ |
|---|---|---|---|
| Group | name | Sequence | ID NO |
| Pluri- | hOCT4_F | GGGCTCTCCCATGCATTCA | 1 |
| potency | | AAC | |
| | hOCT4_R | CACCTTCCCTCCAACCAGT | 2 |
| | | TGC | |
| | hNANOG_F | TGGGATTTACAGGCGTGAG | 3 |
| | | CCAC | |
| | hNANOG_R | AAGCAAAGCCTCCCAATCC | 4 |
| | | CAAAC | |
| Motor | OLIG2_F | GTT CTC CCC TGA GGC | 5 |
| neuron | | TTT TC | |
| precursor | OLIG2_R | AGA AAA AGG TCA TCG | 6 |
| | | GGC TC | |
| Mature | HB9_F | GTC CAC CGC GGG CAT | 7 |
| motor | | GAT CC | |
| neuron | HB9_R | TCT TCA CCT GGG TCT | 8 |
| | | CGG TGA GC | |
| | CHAT_F | GGA GGC GTG GAG CTC | 9 |
| | | AGC GAC ACC | |
| | CHAT_R | CGG GGA GCT CGC TGA | 10 |
| | | CGG AGT CTG | |
| | ACTB_F | TGA AGT GTG ACG TGG | 11 |
| | | ACA TC | |
| | ACTB_R | GGA GGA GCA ATG ATC | 12 |
| | | TTG AT | |

### Immunofluorescence Staining

Cells were fixed in 4% paraformaldehyde for 30 minutes, permeabilized with 0.25% Triton X-100 and blocked with 5% FCS in PBS for 1 hour. The fixed cells were incubated overnight at 4 C in PBS+1% FCS with antibodies against Mouse anti-OCT4 (1:500, ab18976, Abcam), Rabbit anti-SOX2 (1:500, MA516399, ThermoFisher), Rabbit anti-TUJ1 (1:500, MAB 1195, R&D system), Rabbit anti-OLIG2 (1:500, NBP128667, Novus), Rabbit anti-HB9 (1:500, ABN174, Millipore-Sigma), CHAT (1:500, AB144P, Abcam), Cleaved caspase 3 (1:500, 9669S, Cell Signaling Technology), and TDP-43 (1:500, 10782-2AP, Proteintech) followed by incubation with secondary antibodies: FITC-conjugated anti-mouse IgG (1:1000, A21202, Life Technologies), FITC-conjugated anti-rabbit IgG (1:1000, A11034, Life Technologies), Cy3-conjugated anti-mouse IgG (1:1000, A11003, Life Technologies), and Cy3-conjugated anti-rabbit IgG (1:1000, A11035, Life Technologies). The treated cells were covered with slow-fade anti-fade with DAPI (Life Technologies) for nuclear staining and covered with a glass coverslip. Images were captured with a fluorescence microscope (DM5000B, Leica).

### Apoptotic Assay

The apoptotic assay of fibroblasts was by Annexin V-CF Blue/7-AAD Apoptosis Detection Kit (ab214663, Abcam) according to the manufacturer's protocol. Briefly, cells were detached using 0.05% trypsin and washed twice with PBS. Then, samples were resuspended in 1× annexin-binding buffer and incubated with 5 μL Annexin V-FITC and 5 μL 7-amino-actinomycin D (7AAD) for 15 min at 37° C., avoiding light. Finally, the stained samples were analyzed on a Guava Easycyte Flowcytometer (Millipore-Sigma) at an excitation wavelength of 488 nm and emission wavelengths of 525 and 625 nm. For MNs, the apoptotic assay was conducted on two different markers, FITC-conjugated-annexinV (ab201540) or Cleaved caspase 3 (9669S). For annexinV detection, MNs in 96-well plates were washed twice with PBS. Then, 100 μL Annexin V binding buffer was added with 5 μL Annexin V-FITC for 5 min incubation at 37° C., avoiding light. The stained plate was analyzed on a SpectraMax iD3 Multi-Mode Microplate Reader (495/525 nm). For the Cleaved caspase 3, the immunofluorescence protocol was used.

### High-Content Analysis

For differentiation efficiency, analysis neurite outgrowth analysis, and apoptosis analysis, plates were imaged using the high-content imaging system, ImageXpress Micro (IXM, Molecular Devices); a set of 5×5 or 7×7 fields was collected from each well using the 10× or 20× objective. Data were further analyzed in MetaXpress 6 software (Molecular Devices).

### Analysis of Neurite Length

During differentiation into MNs, cells were sampled at 5 day intervals between 20 and 60 days. MNs were cultured in 8-well chamber slides (LabTek II CC2 coated), stained with Tuj1 and imaged by IXM. MetaXpress 6 software was used to analyze the mean process length as a measure of the total outgrowth divided by the number of processes of the cell. Analysis was performed on a total of 4-10 fields and at least 1000 cells per group from three independent experiments. For the neurite size comparison, the data were normalized by the values at day 20; maintenance/shrinkage of neurites over time was determined as net changes from neurite sizes on days 20 and 25.

### Western Blotting

Nuclear and cytoplasmic extracts were prepared from each group with NE-PER™ (ThermoFisher) according to the manufacturer's protocol. Protein quantification was carried out by the BCA (Pierce™ BCA Protein Assay Kit, ThermoFisher) method and the bovine serum protein was taken as the standard. A total of 30 μg of protein were incubated with 4× Laemmli sample buffer for 10 min at 95° C., loaded onto 4-20% SDS-PAGE Mini-PROTEAN TGX Stain-Free™ gels (Bio-Rad Laboratories Inc., Hercules, CA) and separated by gel electrophoresis. Proteins were transferred to polyvinylidene fluoride (PVDF) membranes (Millipore, Billerica, MA, USA) and blocked for 1 h with 10% non-fat milk in Tris-buffered saline with 0.1% Tween 20. Proteins were then blotted with antibodies against TDP-43 (1:1000, Proteintech), PCNA (1:1000, Abcam, ab2426), beta-actin (1:1000, MA5-15739, ThermoFisher). Detection of the primary antibody was accomplished using HRP-conjugated anti-rabbit IgG (1:2000, ab205718, Abcam). Membranes were scanned by ChemiDoc XRS (Bio-Rad) and analyzed by Image Lab 6.1 (Bio-Rad).

### Antibody Array

CMs from pluripotent stem cells or differentiated cells were analyzed on a human L1000 antibody capture array (AAH-BLG-1000, Raybiotech), processed according to the manufacturer's protocol. The array slides were imaged by a Molecular Devices 4000b scanner and data were calculated by Genepix. After normalization by dF-CM, differently changed proteins over 2-fold up- or 0.5-fold down-regulated were selected.

### Protein Cocktail Test

To reproduce and assess the effects of cocktail, recombinant proteins, damaged MNs by H₂O₂ were incubated with recombinant proteins, TIMP1 (200 nM, ab55236, Abcam), Angiogenin (200 nM, 175600, Millipore-Sigma), Angiopoietin-1 (200 nM, 923AN025, R & D system) and Apolipoprotein E (200 nM, ab50244, Abcam) for 24 hours. Then, the viability was tested.

### Bioinformatics Analysis

The gene ID of differently regulated proteins was performed using DAVID Bioinformatics Resources (version 6.8, https://david.ncifcrf.gov), as well as the GO (Gene Ontology) analysis of the differential proteins based on the biological process (BP), molecular function (MF) and cellular component (CC), and Kyoto Encyclopedia of Genes and Genomes (KEGG) pathway. Heat maps were performed with TIGR MeV Ver.4.9 software (Institute of Genomic Research). Violin plots were constructed with the Python seaborn package and normalized with a standard min-max scaler.

### Neurological Scoring

The neurological scoring was performed as published⁴ and criteria are as follows:
Score of 0: While mouse is suspended by the tail, the hindlimbs are fully extended away from the lateral midline and keep the status for two seconds.
Score of 1: Partial collapse of leg extension or trembling of hind legs during tail suspension. This stage is considered an onset of symptoms.
Score of 2: While mouse is suspended by the tail, the hindlimbs not extending much or completely collapsed. During walking of 90 cm, toes curling or dragging of leg parts is observed.
Score of 3: Rigid paralysis or minimal joint movement is observed. Hindlimbs not being used much for forwarding motion. This stage is considered an end-stage.
Score of 4: Mouse cannot right itself within 15 seconds after being placed on either side.

All mice were euthanized when the first signs of paralysis were observed in the hind limbs due to animal welfare issues. So we didn't proceed to a score of 4. All information was recorded for each mouse every other day until the end-stage (a score of 3).

### Body Weights and Behavior Test

In this study, all mice were weighed every other day.

For hanging test, mice were placed on a grid and then turned upside down. Then, the latency to fall was measured. The latency time measurements began from the point when the mouse was hanging free on the grid and ended with the animal falling to the cage underneath the grid.

### Survival

For survival assessment, 6 female (3 ALS iPSC-CM group and 3 dF-CM group) and 6 male (3 ALS iPSC-CM group and 3 dF-CM group) SOD1^{G93A} mice were used. It was considered that animals reached the end point of the disease when they showed any first signs of paralysis.

### Tissue Collection

When mice showed the first signs of paralysis in hind limbs or 100 days or 130 days, euthanasia was proceeded with the guidelines of UC Berkeley's OLAC administration. For histological comparison, some ALS mice were euthanized according to the end-stage of dF-CM mice. Spinal cords and muscles (Gastrocnemius and Tibialis anterior) were isolated. Then, spinal cords were fixed with 4% paraformaldehyde (PFA) overnight at 4° C. and muscle tissues were fixed for 30 min at room temperature. Then, all tissues were transferred to 30% sucrose solution overnight and embedded in tissue-tek optimal cutting temperature (OCT, Sakura Finetek, The Netherlands) and snap frozen in isopentane cooled to −70° C. with dry ice.

For histological analysis, all OCT-embedded tissues were cryosectioned by a Cryostar NX50 (ThermoFisher). Muscle tissues were sectioned to 20 μm thickness while spinal cords were obtained at 10 μm.

### Muscle Weight

To compare the wet muscle weight of the GA and TA in all groups, mice were euthanized and muscle tissues were isolated, and both left and right muscles were weighed on a scale. For the comparison, all weight recorded was normalized by their body weight.

### Cresyl Violet Staining and Quantification

Mounted frozen sections were dried overnight at 37° C. and placed slides directly into 1:1 alcohol/chloroform overnight, then rehydrate through 100% alcohol to distilled water. Next, sections were stained in 0.1% cresyl violet solution for 5-10 minutes at 37c, rinsed with distilled water, dehydrated with ethanol and cleared in xylene, and covered with mount media (ThermoFisher). Stained sections were imaged at 5× and 10× magnification. For quantification, five sections were selected randomly and stained MNs in the ventral spinal cord were counted.

### Neuromuscular Junction Staining and Quantification

Neuromuscular junction (NMJ) analysis was performed as previously described s. Briefly, dried sections were permeabilized with 1% Triton X-100 and blocked with 5% FBS in PBS for 1 hour. The fixed cells were incubated for 2 hours at room temperature in PBS+1% FBS with antibodies, Alex 488-conjugated neurofilament (1:500, 8024, Cell Signaling Technology), Alex 488-conjugated synaptophysin (1:500, MAB5258A4, Millipore-Sigma), and Alex 555-conjugated a-bungarotoxin (1:500, B35451, Life Technologies). The stained slides were covered with slow-fade anti-fade with DAPI (Life Technologies) for nuclear staining and covered with a glass coverslip. Images were captured with a fluorescence microscope (DM5000B, Leica). For quantification, five sections were selected randomly and counted. Intact NMJs (yellow) were identified as those that exhibited an overlay of a-bungarotoxin (red) and neurofilament (green). Denervated NMJs were defined by labeling with only a-bungarotoxin (red). Percent intact NMJs was calculated as the number of intact NMJs (yellow)/total number of motor end plates (yellow+red).

### Statistical Analysis

All statistical analyses were performed using GraphPad Prism software version 5 (GraphPad software Inc). All values are expressed as means±SEM for independent experiments, or SD for replicates. To determine the significance of differences among groups, comparisons were made using Student's t-test. Survival and onset data was analyzed with Kaplan-Meier curves and log rank test. The P<0.05 was considered significant.

### REFERENCES AND NOTES FOR EXAMPLE 1

1. W. Robberecht, T. Philips, The changing scene of amyotrophic lateral sclerosis. Nat. Rev. Neurosci. 14 (2013), pp. 248-264.
2. A. Al-Chalabi, A. Jones, C. Troakes, A. King, S. Al-Sarraj, L. H. Van Den Berg, The genetics and neuropathology of amyotrophic lateral sclerosis. Acta Neuropathol. 124 (2012), pp. 339-352.
3. M. E. Gurney, H. Pu, A. Y. Chiu, M. C. Dal Canto, C. Y. Polchow, D. D. Alexander, J. Caliendo, A. Hentati, Y. W. Kwon, H. X. Deng, W. Chen, P. Zhai, R. L. Sufit, T. Siddique, Motor neuron degeneration in mice that express a human Cu,Zn superoxide dismutase mutation. Science (80-). 264, 1772-1775 (1994).
4. D. R. Borchelt, M. K. Lee, H. S. Slunt, M. Guarnieri, Z. S. Xu, P. C. Wong, R. H. Brown, D. L. Price, S. S. Sisodia, D. W. Cleveland, Superoxide dismutase 1 with mutations linked to familial amyotrophic lateral sclerosis possesses significant activity. Proc. Natl. Acad. Sci. U.S.A 91, 8292-8296 (1994).
5. A. Nordlund, L. Leinartaite, K. Saraboji, C. Aisenbrey, G. Grobner, P. Zetterström, J. Danielsson, D. T. Logan, M. Oliveberg, Functional features cause misfolding of the ALS-provoking enzyme SOD1. Proc. Natl. Acad. Sci. U.S.A 106, 9667-9672 (2009).
6. A. C. Estévez, J. P. Crow, J. B. Sampson, C. Reiter, Y. Zhuang, G. J. Richardson, M. M. Tarpey, L. Barbeito, J. S. Beckman, Induction of nitric oxide-dependent apoptosis in motor neurons by zinc-deficient superoxide dismutase. Science (80-). 286, 2498-2500 (1999).
7. M. S. Rotunno, D. A. Bosco, An emerging role for misfolded wild-type SOD1 in sporadic ALS pathogenesis. Front. Cell. Neurosci. 7 (2013), p. 253.
8. D. A. Bosco, G. Morfini, N. M. Karabacak, Y. Song, F. Gros-Louis, P. Pasinelli, H. Goolsby, B. A. Fontaine, N. Lemay, D. McKenna-Yasek, M. P. Frosch, J. N. Agar, J. P. Julien, S. T. Brady, R. H. Brown, Wild-type and mutant SOD1 share an aberrant conformation and a common pathogenic pathway in ALS. Nat. Neurosci. 13, 1396-1403 (2010).
9. R. Rakhit, P. Cunningham, A. Furtos-Matei, S. Dahan, X. F. Qi, J. P. Crow, N. R. Cashman, L. H. Kondejewski, A. Chakrabartty, Oxidation-induced misfolding and aggregation of superoxide dismutase and its implications for amyotrophic lateral sclerosis. J. Biol. Chem. 277, 47551-47556 (2002).
10. S. A. Ezzi, M. Urushitani, J.-P. Julien, Wild-type superoxide dismutase acquires binding and toxic properties of ALS-linked mutant forms through oxidation. J. Neurochem. 102, 170-178 (2007).
11. S. C. Barber, R. J. Mead, P. J. Shaw, Oxidative stress in ALS: A mechanism of neurodegeneration and a therapeutic target. Biochim. Biophys. Acta—Mol. Basis Dis. 1762 (2006), pp. 1051-1067.
12. K. Aoyama, T. Nakaki, Impaired Glutathione Synthesis in Neurodegeneration. Int. J. Mol. Sci. 14, 21021-21044 (2013).
13. M. R. Vargas, D. A. Johnson, J. A. Johnson, Decreased glutathione accelerates neurologiCal deficit and mitochondrial pathology in familial ALS-linked hSOD1G93A mice model. Neurobiol. Dis. 43, 543-551 (2011).
14. A. Prasad, V. Bharathi, V. Sivalingam, A. Girdhar, B. K. Patel, Molecular mechanisms of TDP-43 misfolding and pathology in amyotrophic lateral sclerosis. Front. Mol. Neurosci. 12, 1-36 (2019).
15. G. Kleinberger, H. Wils, P. Ponsaerts, G. Joris, J. P. Timmermans, C. Van Broeckhoven, S. Kumar-Singh, Increased caspase activation and decreased TDP-43 solubility in progranulin knockout cortical cultures. J. Neurochem. 115, 735-747 (2010).
16. M. A. Vogt, Z. Ehsaei, P. Knuckles, A. Higginbottom, M. S. Helmbrecht, T. Kunath, K. Eggan, L. A. Williams, P. J. Shaw, W. Wurst, T. Floss, A. B. Huber, V. Taylor, TDP-43 induces p53-mediated cell death of cortical progenitors and immature neurons. Sci. Rep. 8, 8097 (2018).
17. P. Baskaran, C. Shaw, S. Guthrie, TDP-43 causes neurotoxicity and cytoskeletal dysfunction in primary cortical neurons. PLoS One. 13, e0196528 (2018).
18. Y. J. Liu, T. C. Ju, H. M. Chen, Y. S. Jang, L. M. Lee, H. L. Lai, H. C. Tai, J. M. Fang, Y. L. Lin, P. H. Tu, Y. Chern, Activation of AMP-activated protein kinase al mediates mislocalization of TDP-43 in amyotrophic lateral sclerosis. Hum. Mol. Genet. 24, 787-801 (2015).
19. I. R. A. Mackenzie, E. H. Bigio, P. G. Ince, F. Geser, M. Neumann, N. J. Cairns, L. K. Kwong, M. S. Forman, J. Ravits, H. Stewart, A. Eisen, L. McClusky, H. A. Kretzschmar, C. M. Monoranu, J. R. Highley, J. Kirby, T. Siddique, P. J. Shaw, V. M. Y. Lee, J. Q. Trojanowski, Pathological TDP-43 distinguishes sporadic amyotrophic lateral sclerosis from amyotrophic lateral sclerosis with SOD1 mutations. Ann. Neurol. 61, 427-434 (2007).
20. C. F. Tan, H. Eguchi, A. Tagawa, 0. Onodera, T. Iwasaki, A. Tsujino, M. Nishizawa, A. Kakita, H. Takahashi, TDP-43 immunoreactivity in neuronal inclusions in familial amyotrophic lateral sclerosis with or without SOD1 gene mutation. Acta Neuropathol. 113, 535-542 (2007).
21. M. Neumann, D. M. Sampathu, L. K. Kwong, A. C. Truax, M. C. Micsenyi, T. T. Chou, J. Bruce, T. Schuck, M. Grossman, C. M. Clark, L. F. McCluskey, B. L. Miller, E. Masliah, I. R. Mackenzie, H. Feldman, W. Feiden, H. A. Kretzschmar, J. Q. Trojanowski, V. M. Y. Lee, Ubiquitinated TDP-43 in frontotemporal lobar degeneration and amyotrophic lateral sclerosis. Science (80-). 314, 130-133 (2006).
22. T. Arai, M. Hasegawa, H. Akiyama, K. Ikeda, T. Nonaka, H. Mori, D. Mann, K. Tsuchiya, M. Yoshida, Y. Hashizume, T. Oda, TDP-43 is a component of ubiquitin-positive tau-negative inclusions in frontotemporal lobar degeneration and amyotrophic lateral sclerosis. Biochem. Biophys. Res. Commun. 351, 602-611 (2006).
23. A. M. Blokhuis, E. J. N. Groen, M. Koppers, L. H. Van Den Berg, R. J. Pasterkamp, Protein aggregation in amyotrophic lateral sclerosis. Acta Neuropathol. 125 (2013), pp. 777-794.
24. H.-X. Deng, H. Zhai, E. H. Bigio, J. Yan, F. Fecto, K. Ajroud, M. Mishra, S. Ajroud-Driss, S. Heller, R. Sufit, N. Siddique, E. Mugnaini, T. Siddique, Ann. Neurol., in press, doi:10.1002/ana.22051.
25. M. D. Cai, K. W. Lee, S. M. Choi, E. J. Yang, TDP-43 modification in the hSOD1G93A amyotrophic lateral sclerosis mouse model. Neurol. Res. 37, 253-262 (2015).
26. Y. Lu, C. Tang, L. Zhu, J. Li, H. Liang, J. Zhang, R. Xu, The overexpression of TDP-43 protein in the neuron and oligodendrocyte cells causes the progressive motor neuron degeneration in the SOD1 G93A transgenic mouse model of amyotrophic lateral sclerosis. Int. J. Biol. Sci. 12, 1140-1149 (2016).
27. K. Forsberg, P. A. Jonsson, P. M. Andersen, D. Bergemalm, K. S. Graffmo, M. Hultdin, J. Jacobsson, R. Rosquist, S. L. Marklund, T. Brannstrom, Novel Antibodies Reveal Inclusions Containing Non-Native SOD1 in Sporadic ALS Patients. PLoS One. 5, el 1552 (2010).
28. G. S. Jeon, Y. M. Shim, D. Y. Lee, J. S. Kim, M. J. Kang, S. H. Ahn, J. Y. Shin, D. Geum, Y. H. Hong, J. J. Sung, Pathological Modification of TDP-43 in Amyotrophic Lateral Sclerosis with SOD1 Mutations. Mol. Neurobiol. 56, 2007-2021 (2019).
29. W. Zakrzewski, M. Dobrzynski, M. Szymonowicz, Z. Rybak, Stem cells: Past, present, and future. Stem Cell Res. Ther. 10 (2019), p. 68.
30. I. Gutierrez-Aranda, V. Ramos-Mejia, C. Bueno, M. Munoz-Lopez, P. J. Real, A. Mácia, L. Sanchez, G. Ligero, J. L. Garcia-Parez, P. Menendez, Human induced pluripotent stem cells develop teratoma more efficiently and faster than human embryonic stem cells regardless the site of injection. Stem Cells. 28 (2010), pp. 1568-1570.
31. H. Fukuoka, H. Suga, K. Narita, R. Watanabe, S. Shintani, The Latest Advance in Hair Regeneration Therapy Using Proteins Secreted by Adipose-Derived Stem Cells. Am. J. Cosmet. Surg. 29, 273-282 (2012).
32. S. H. Bhang, S. Lee, J. Y. Shin, T. J. Lee, H. K. Jang, B. S. Kim, Efficacious and clinically relevant conditioned medium of human adipose-derived stem cells for therapeutic angiogenesis. Mol. Ther. 22, 862-872 (2014).
33. M. N. M. Walter, K. T. Wright, H. R. Fuller, S. MacNeil, W. E. B. Johnson, Mesenchymal stem cell-conditioned medium accelerates skin wound healing: An in vitro study of fibroblast and keratinocyte scratch assays. Exp. Cell Res. 316, 1271-1281 (2010).
34. L. Timmers, S. K. Lim, I. E. Hoefer, F. Arslan, R. C. Lai, A. A. M. van Oorschot, M. J. Goumans, C. Strijder, S. K. Sze, A. Choo, J. J. Piek, P. A. Doevendans, G. Pasterkamp, D. P. V. de Kleijn, Human mesenchymal stem cell-conditioned medium improves cardiac function following myocardial infarction. Stem Cell Res. 6, 206-214 (2011).
35. S. K. Sze, D. P. V. de Kleijn, R. C. Lai, E. K. W. Tan, H. Zhao, K. S. Yeo, T. Y. Low, Q. Lian, C. N. Lee, W. Mitchell, R. M. El Oakley, S. K. Lim, Elucidating the secretion proteome of human embryonic stem cell-derived mesenchymal stem cells. Mol. Cell. Proteomics. 6, 1680-1689 (2007).
36. D. Cantinieaux, R. Quertainmont, S. Blacher, L. Rossi, T. Wanet, A. Noel, G. Brook, J. Schoenen, R. Franzen, Conditioned Medium from Bone Marrow-Derived Mesenchymal Stem Cells Improves Recovery after Spinal Cord Injury in Rats: An Original Strategy to Avoid Cell Transplantation. PLoS One. 8 (2013), doi:10.1371/journal.pone.0069515.
37. C. V. Fontanilla, H. Gu, Q. Liu, T. Z. Zhu, C. Zhou, B. H. Johnstone, K. L. March, R. M. Pascuzzi, M. R. Farlow, Y. Du, Adipose-derived Stem Cell Conditioned Media Extends Survival time of a mouse model of Amyotrophic Lateral Sclerosis. Sci. Rep. 5, 1-11 (2015).
38. B. R. Zhou, Y. Xu, Y. Xu, S. L. Guo, Y. Wang, F. Zhu, F. Permatasari, D. Wu, Z. Q. Yin, D. Luo, The effect of conditioned media of Adipose-derived stem cells on wound healing after ablative fractional carbon dioxide laser resurfacing. Biomed Res. Int. 2013 (2013), doi:10.1155/2013/519126.
39. G. Sagaradze, O. Grigorieva, P. Nimiritsky, N. Basalova, N. Kalinina, Z. Akopyan, A. Efimenko, Conditioned medium from human mesenchymal stromal cells: Towards the clinical translation. Int. J. Mol. Sci. 20 (2019), doi:10.3390/ijms20071656.
40. S. Neel, D. K. Singla, Induced pluripotent stem (iPS) cells inhibit apoptosis and fibrosis in streptozotocin-induced diabetic rats. Mol. Pharm. 8, 2350-2357 (2011).
41. Y. Zhang, C. Deng, J. Qian, M. Zhang, X. Li, Improvement of radiotherapy-induced lacrimal gland injury by induced pluripotent stem cell-derived conditioned medium via MDK and inhibition of the p38/JNK pathway. Int. J. Mol. Sci. 15, 18407-18421 (2014).
42. M. Oh, Y. J. Kim, Y. J. Son, H. S. Yoo, J. H. Park, Promotive Effects of Human Induced Pluripotent Stem Cell-conditioned Medium on the Proliferation and Migration of Dermal Fibroblasts. Biotechnol. Bioprocess Eng. 22, 561-568 (2017).
43. H. Yousef, M. J. Conboy, J. Li, M. Zeiderman, T. Vazin, C. Schlesinger, D. V. Schaffer, I. M. Conboy, hESC-secreted proteins can be enriched for multiple regenerative therapies by heparin-binding. Aging (Albany. NY). 5, 357-372 (2013).
44. H. Yousef, M. J. Conboy, H. Mamiya, M. Zeiderman, C. Schlesinger, D. V. Schaffer, I. M. Conboy, Mechanisms of action of hESC-secreted proteins that enhance human and mouse myogenesis. Aging (Albany. NY). 6, 602-620 (2014).
45. D. Yang, W. Wang, L. Li, Y. Peng, P. Chen, H. Huang, Y. Guo, X. Xia, Y. Wang, H. Wang, W. E. Wang, C. Zeng, The Relative Contribution of Paracine Effect versus Direct Differentiation on Adipose-Derived Stem Cell Transplantation Mediated Cardiac Repair. PLoS One. 8 (2013), doi:10.1371/journal.pone.0059020.
46. M. Takahashi, T.-S. Li, R. Suzuki, T. Kobayashi, H. Ito, Y. Ikeda, M. Matsuzaki, K. Hamano, T. Koba-yashi, Cytokines produced by bone marrow cells can contribute to functional improvement of the infarcted heart by protecting cardiomyocytes from ischemic injury. Am J Physiol Hear. Circ Physiol. 291, 886-893 (2006).
47. L. Comley, I. Allodi, S. Nichterwitz, M. Nizzardo, C. Simone, S. Corti, E. Hedlund, Motor neurons with differential vulnerability to degeneration show distinct protein signatures in health and ALS. Neuroscience. 291, 216-229 (2015).
48. J. H. Park, H. S. Park, S. Hong, S. Kang, Motor neurons derived from ALS-related mouse iPS cells recapitulate pathological features of ALS. Exp. Mol. Med. 48, e276 (2016).
49. S. Karumbayaram, T. K. Kelly, A. A. Paucar, A. J. T. Roe, J. A. Umbach, A. Charles, S. A. Goldman, H. I. Kornblum, M. Wiedau-Pazos, Human embryonic stem cell-derived motor neurons expressing SOD1 mutants exhibit typical signs of motor neuron degeneration linked to ALS. DMM Dis. Model. Mech. 2, 189-195 (2009).
50. K. Fujimori, M. Ishikawa, A. Otomo, N. Atsuta, R. Nakamura, T. Akiyama, S. Hadano, M. Aoki, H. Saya, G. Sobue, H. Okano, Modeling sporadic ALS in iPSC-derived motor neurons identifies a potential therapeutic agent. Nat. Med. 24, 1579-1589 (2018).
51. H. Chen, K. Qian, Z. Du, J. Cao, A. Petersen, H. Liu, L. W. Blackbourn, C. L. Huang, A. Errigo, Y. Yin, J. Lu, M. Ayala, S. C. Zhang, Modeling ALS with iPSCs reveals that mutant SOD1 misregulates neurofilament balance in motor neurons. Cell Stem Cell. 14, 796-809 (2014).
52. M. M. Haulcomb, N. A. Mesnard, R. J. Batka, T. D. Alexander, V. M. Sanders, K. J. Jones, Axotomy-induced target disconnection promotes an additional death mechanism involved in motoneuron degeneration in amyotrophic lateral sclerosis transgenic mice. J. Comp. Neurol. 522, 2349-2376 (2014).
53. M. Li, V. O. Ona, C. Gudgan, M. Chen, V. Jackson-Lewis, L. J. Andrews, A. J. Olszewski, P. E. Stieg, J. P. Lee, S. Przedborski, R. M. Friedlander, Functional role of caspase-1 and caspase-3 in an ALS transgenic mouse model. Science (80-). 288, 335-339 (2000).
54. L. J. MARTIN, Neuronal Death in Amyotrophic Lateral Sclerosis Is Apoptosis. J. Neuropathol. Exp. Neurol. 58, 459-471 (1999).
55. V. Kostic, V. Jackson-Lewis, F. De Bilbao, M. Dubois-Dauphin, S.

Przedborski, Bcl-2: Prolonging life in a transgenic mouse model of familial amyotrophic lateral sclerosis. Science (80-). 277, 559-562 (1997).
56. C. L. Bigarella, R. Liang, S. Ghaffari, Stem cells and the impact of ROS signaling. Dev. 141, 4206-4218 (2014).
57. D.-C. Tarng, W.-C. Tseng, P.-Y. Lee, S.-H. Chiou, S.-L. Hsieh, Induced Pluripotent Stem Cell-Derived Conditioned Medium Attenuates Acute Kidney Injury by Downregulating the Oxidative Stress-Related Pathway in Ischemia-Reperfusion Rats. Cell Transplant. 25, 517-530 (2016).
58. X. Paez-Colasante, C. Figueroa-Romero, S. A. Sakowski, S. A. Goutman, E. L. Feldman, Amyotrophic lateral sclerosis: Mechanisms and therapeutics in the epigenomic era. Nat. Rev. Neurol. 11(2015), pp. 266-279.
59. L. Ferraiuolo, J. Kirby, A. J. Grierson, M. Sendtner, P. J. Shaw, Molecular pathways of motor neuron injury in amyotrophic lateral sclerosis. Nat. Rev. Neurol. 7, 616-630(2011).
60. D. Shimojo, K. Onodera, Y. Doi-Torii, Y. Ishihara, C. Hattori, Y. Miwa, S. Tanaka, R. Okada, M. Ohyama, M. Shoji, A. Nakanishi, M. Doyu, H. Okano, Y. Okada, Rapid, efficient, and simple motor neuron differentiation from human pluripotent stem cells. Mol. Brain. 8, 79 (2015).
61. Q. Qu, D. Li, K. R. Louis, X. Li, H. Yang, Q. Sun, S. R. Crandall, S. Tsang, J. Zhou, C. L. Cox, J. Cheng, F. Wang, High-efficiency motor neuron differentiation from human pluripotent stem cells and the function of Islet-1. Nat. Commun. 5 (2014), doi:10.1038/ncomms4449.
62. Y. Wang, L. Zhang, Y. Li, L. Chen, X. Wang, W. Guo, X. Zhang, G. Qin, S. H. He, A. Zimmerman, Y. Liu, I. M. Kim, N. L. Weintraub, Y. Tang, Exosomes/microvesicles from induced pluripotent stem cells deliver cardioprotective miRNAs and prevent cardiomyocyte apoptosis in the ischemic myocardium. Int. J. Cardiol. 192, 61-69 (2015).
63. K. Sakamoto, K. Karelina, K. Obrietan, CREB: A multifaceted regulator of neuronal plasticity and protection. J. Neurochem. 116, 1-9 (2011).
64. E. Tejima, S. Guo, Y. Murata, K. Arai, J. Lok, K. Van Leyen, A. Rosell, X. Wang, E. H. Lo, Neuroprotective effects of overexpressing tissue inhibitor of metalloproteinase TIMP-1. J. Neurotrauma (2009), doi:10.1089/neu.2009.0959.
65. Ashutosh, C. Chao, K. Borgmann, K. Brew, A. Ghorpade, Tissue inhibitor of metalloproteinases-1 protects human neurons from staurosporine and HIV-1-induced apoptosis: Mechanisms and relevance to HIV-1-associated dementia. Cell Death Dis. 3, 1-9 (2012).
66. H. D. Huynh, J. Zheng, M. Umikawa, C. Zhang, R. Silvany, S. lizuka, M. Holzenberger, W. Zhang, C. C. Zhang, IGF binding protein 2 supports the survival and cycling of hematopoietic stem cells. Blood. 118, 3236-3243 (2011).
67. T. Migita, T. Narita, R. Asaka, E. Miyagi, H. Nagano, K. Nomura, M.

Matsuura, Y. Satoh, S. Okumura, K. Nakagawa, H. Seimiya, Y. Ishikawa, Role of insulin-like growth factor binding protein 2 in lung adenocarcinoma: IGF-independent antiapoptotic effect via caspase-3. Am. J. Pathol. 176, 1756-1766 (2010).
68. L. Fletcher, E. Isgor, S. Sprague, L. H. Williams, B. B. Alajajian, D. F. Jimenez, M. Digicaylioglu, Spatial distribution of insulin-like growth factor binding protein-2 following hypoxic-ischemic injury. BMC Neurosci. (2013), doi:10.1186/1471-2202-14-158.
69. T. D. Stein, N. J. Anders, C. DeCarli, S. L. Chan, M. P. Mattson, J. A. Johnson, Neutralization of transthyretin reverses the neuroprotective effects of secreted amyloid precursor protein (APP) in APPSw mice resulting in tau phosphorylation and loss of hippocampal neurons: Support for the amyloid hypothesis. J. Neurosci. 24, 7707-7717 (2004).
70. J. Y. Huang, Y. T. Hong, J. I. Chuang, Fibroblast growth factor 9 prevents MPP+-induced death of dopaminergic neurons and is involved in melatonin neuroprotection in vivo and in vitro. J. Neurochem. 109, 1400-1412 (2009).
71. S. Valable, A. Bellail, S. Lesné, G. Liot, E. T. MacKenzie, D. Vivien, M. Bernaudin, E. Petit, Angiopoietin-1-induced phosphatidyl-inositol 3-kinase activation prevents neuronal apoptosis. FASEB J. 17, 1-19 (2003).
72. V. Subramanian, B. Crabtree, K. R. Acharya, Human angiogenin is a neuroprotective factor and amyotrophic lateral sclerosis associated angiogenin variants affect neurite extension/pathfinding and survival of motor neurons. Hum. Mol. Genet. 17, 130-149 (2008).
73. S. Zhou, H. Wu, C. Zeng, X. Xiong, S. Tang, Z. Tang, X. Sun, Apolipoprotein e protects astrocytes from hypoxia and glutamate-induced apoptosis. FEBS Lett. 587, 254-258 (2013).
74. R. L. Lian, X. L. Guo, J. S. Chen, Y. L. Guo, J. F. Zheng, Y. W. Chen, Effects of induced pluripotent stem cells-derived conditioned medium on the proliferation and anti-apoptosis of human adipose-derived stem cells. Mol. Cell. Biochem. 413, 69-85 (2016).
75. X. Lu, D. Chen, Z. Liu, C. Li, Y. Liu, J. Zhou, P. Wan, Y. gao Mou, Z. Wang, Enhanced survival in vitro of human corneal endothelial cells using mouse embryonic stem cell conditioned medium. Mol. Vis. 16, 611-622 (2010).
76. P. B. Verghese, J. M. Castellano, K. Garai, Y. Wang, H. Jiang, A. Shah, G. Bu, C. Frieden, D. M. Holtzman, ApoE influences amyloid-β (Aβ) clearance despite minimal apoE/Aβ association in physiological conditions. Proc. Natl. Acad. Sci. U.S.A 110, E1807-E1816 (2013).
77. A. P. Arrigo, S. Virot, S. Chaufour, W. Firdaus, C. Kretz-Remy, C. Diaz-Latoud, Hsp27 consolidates intracellular redox homeostasis by upholding glutathione in its reduced form and by decreasing iron intracellular levels. Antioxidants Redox Signal. 7 (2005), pp. 414-424.
78. P. Wang, C. M. Wander, C. X. Yuan, M. S. Bereman, T. J. Cohen, Acetylation-induced TDP-43 pathology is suppressed by an HSF1-dependent chaperone program. Nat. Commun. 8, 1-15 (2017).
79. P. Pandey, R. Farber, A. Nakazawa, S. Kumar, A. Bharti, C. Nalin, R. Weichselbaum, D. Kufe, S. Kharbanda, Hsp27 functions as a negative regulator of cytochrome c-dependent activation of procaspase-3. Oncogene. 19, 1975-1981 (2000).
80. U. K. Jinwal, J. F. Abisambra, J. Zhang, S. Dharia, J. C. O'Leary, T. Patel, K. Braswell, T. Jani, J. E. Gestwicki, C. A. Dickey, Cdc37/Hsp90 protein complex disruption triggers an autophagic clearance cascade for TDP-43 protein. J. Biol. Chem. 287, 24814-24820 (2012).
81. R. Baumann, C. Casaulta, D. Simon, S. Conus, S. Yousefi, H.-U. Simon, Macrophage migration inhibitory factor delays apoptosis in neutrophils by inhibiting the mitochondria-dependent death pathway. FASEB J. 17, 2221-2230 (2003).
82. N. Agata, R. Ahmad, T. Kawano, D. Raina, S. Kharbanda, D. Kufe, MUC1 oncoprotein blocks death receptor-mediated apoptosis by inhibiting recruitment of caspase-8. Cancer Res. 68, 6136-6144 (2008).
83. Y. Han, B. Ripley, S. Serada, T. Naka, M. Fujimoto, Interleukin-6 Deficiency Does Not Affect Motor Neuron Disease Caused by Superoxide Dismutase 1 Mutation. PLoS One. 11, e0153399 (2016).
84. T. Hatzipetros, J. D. Kidd, A. J. Moreno, K. Thompson, A. Gill, F. G. Vieira, A quick phenotypic neurological scoring system for evaluating disease progression in the SOD1-G93A mouse model of ALS. J. Vis. Exp. 2015, 53257 (2015).
85. V. Cappello, M. Francolini, Neuromuscular junction dismantling in amyotrophic lateral sclerosis. Int. J. Mol. Sci. 18 (2017), doi:10.3390/ijms18102092.
86. J. A. Clark, K. A. Southam, C. A. Blizzard, A. E. King, T. C. Dickson, Axonal degeneration, distal collateral branching and neuromuscular junction architecture alterations occur prior to symptom onset in the SOD1G93A mouse model of amyotrophic lateral sclerosis. J. Chem. Neuroanat. 76, 35-47 (2016).
87. G. Bruneteau, S. Bauché, J. L. Gonzalez de Aguilar, G. Brochier, N. Mandjee, M.-L. Tanguy, G. Hussain, A. Behin, F. Khiami, E. Sariali, C. Hell-Remy, F. Salachas, P.-F. Pradat, L. Lacomblez, S. Nicole, B. Fontaine, M. Fardeau, J.-P. Loeffler, V. Meininger, E. Fournier, J. Koenig, D. Hantaï, Endplate denervation correlates with Nogo-A muscle expression in amyotrophic lateral sclerosis patients. Ann. Clin. Transl. Neurol. 2, 362-372 (2015).
88. B. A. Berning, A. K. Walker, The pathobiology of TDP-43 C-terminal fragments in ALS and FTLD. Front. Neurosci. 13 (2019), p. 335.
89. Y. Zhang, Y. Wei, D. Liu, F. Liu, X. Li, L. Pan, Y. Pang, D. Chen, Role of growth differentiation factor 11 in development, physiology and disease. Oncotarget. 8 (2017), pp. 81604-81616.
90. R. Derynck, E. H. Budi, Specificity, versatility, and control of TGF-b family signaling. Sci. Signal. 12 (2019), doi:10.1126/scisignal.aav5183.
91. X. Anqi, C. Ruiqi, R. Yanming, Y. Chao, Neuroprotective potential of GDF11 in experimental intracerebral hemorrhage in elderly rats. J. Clin. Neurosci. 63, 182-188 (2019).
92. H. H. Wu, S. Ivkovic, R. C. Murray, S. Jaramillo, K. M. Lyons, J. E. Johnson, A. L. Calof, Autoregulation of neurogenesis by GDF11. Neuron. 37, 197-207 (2003).
93. W. Li, W. Sun, Y. Zhang, W. Wei, R. Ambasudhan, P. Xia, M. Talantova, T. Lin, J. Kim, X. Wang, W. R. Kim, S. A. Lipton, K. Zhang, S. Ding, Rapid induction and long-term self-renewal of primitive neural precursors from human embryonic stem cells by small molecule inhibitors. Proc. Natl. Acad. Sci. U.S.A 108, 8299-8304 (2011).
94. J. R. Smith, L. Vallier, G. Lupo, M. Alexander, W. A. Harris, R. A.

Pedersen, Inhibition of Activin/Nodal signaling promotes specification of human embryonic stem cells into neuroectoderm. Dev. Biol. 313, 107-117 (2008).
95. Y. Iwasaki, T. Shiojima, N. Tagaya, T. Kobayashi, M. Kinoshita, Effect of transforming growth factor β1 on spinal motor neurons after axotomy. J. Neurol. Sci. 147, 9-12 (1997).
96. S. C. Bendall, C. Hughes, J. L. Campbell, M. H. Stewart, P. Pittock, S. Liu, E. Bonneil, P. Thibault, M. Bhatia, G. A. Lajoie, An enhanced mass spectrometry approach reveals human embryonic stem cell growth factors in culture. Mol. Cell. Proteomics. 8, 421-432 (2009).
97. D. Gonzalez, O. Contreras, D. L. Rebolledo, J. P. Espinoza, B. van Zundert, E. Brandan, ALS skeletal muscle shows enhanced TGF-β signaling, fibrosis and induction of fibro/adipogenic progenitor markers. PLoS One. 12, e0177649 (2017).
98. H. Yousef, M. J. Conboy, A. Morgenthaler, C. Schlesinger, L. Bugaj, P. Paliwal, C. Greer, I. M. Conboy, D. Schaffer, Systemic attenuation of the TGF-β pathway by a single drug simultaneously rejuvenates hippocampal neurogenesis and myogenesis in the same old mammal. Oncotarget (2015), doi:10.18632/oncotarget.3851.
99. M. Mehdipour, J. Etienne, C. C. Chen, R. Gathwala, M. Rehman, C. Kato, C. Liu, Y. Liu, Y. Zuo, M. J. Conboy, I. M. Conboy, Rejuvenation of brain, liver and muscle by simultaneous pharmacological modulation of two signaling determinants, that change in opposite directions with age. Aging (Albany. NY). 11, 5628-5645 (2019).
100. Y. L. Guo, S. Chakraborty, S. S. Rajan, R. Wang, F. Huang, Effects of oxidative stress on mouse embryonic stem cell proliferation, apoptosis, senescence, and self-renewal. Stem Cells Dev. 19, 1321-1331 (2010).
101. J. Zhang, I. Khvorostov, J. S. Hong, Y. Oktay, L. Vergnes, E. Nuebel, P. N. Wahjudi, K. Setoguchi, G. Wang, A. Do, H.-J. Jung, J. M. McCaffery, I. J. Kurland, K. Reue, W.-N. P. Lee, C. M. Koehler, M. A. Teitell, UCP2 regulates energy metabolism and differentiation potential of human pluripotent stem cells. EMBO J. 30, 4860-4873 (2011).
102. S. Mandal, A. G. Lindgren, A. S. Srivastava, A. T. Clark, U. Banerjee, Mitochondrial Function Controls Proliferation and Early Differentiation Potential of Embryonic Stem Cells. Stem Cells. 29, 486-495 (2011).
103. W. Zhou, M. Choi, D. Margineantu, L. Margaretha, J. Hesson, C. Cavanaugh, C. A. Blau, M. S. Horwitz, D. Hockenbery, C. Ware, H. Ruohola-Baker, HIF1α induced switch from bivalent to exclusively glycolytic metabolism during ESC-to-EpiSC/hESC transition. EMBO J. 31, 2103-2116 (2012).
104. H. Inoue, K. Tsukita, T. Iwasato, Y. Suzuki, M. Tomioka, M. Tateno, M. Nagao, A. Kawata, T. C. Saido, M. Miura, H. Misawa, S. Itohara, R. Takahashi, The crucial role of caspase-9 in the disease progression of a transgenic ALS mouse model. EMBO J. (2003), doi:10.1093/emboj/cdg634.
105. W. A. Pedersen, H. Luo, I. Kruman, E. Kasarskis, M. P. Mattson, The prostate apoptosis response-4 protein participates in motor neuron degeneration in amyotrophic lateral sclerosis. FASEB J. (2000), doi:10.1096/fasebj.14.7.913.
106. E. N. Guerrero, J. Mitra, H. Wang, S. Rangaswamy, P. M. Hegde, P. Basu, K. S. Rao, M. L. Hegde, Amyotrophic lateral sclerosis-associated TDP-43 mutation Q331K prevents nuclear translocation of XRCC4-DNA ligase 4 complex and is linked to genome damage-mediated neuronal apoptosis. Hum. Mol. Genet. (2019), doi:10.1093/hmg/ddz062.
107. S. Sathasivam, P. J. Shaw, Apoptosis in amyotrophic lateral sclerosis—What is the evidence? Lancet Neurol. 4, 500-509 (2005).
108. F. Soldner, J. Laganière, A. W. Cheng, D. Hockemeyer, Q. Gao, R. Alagappan, V. Khurana, L. I. Golbe, R. H. Myers, S. Lindquist, L. Zhang, D. Guschin, L. K. Fong, B. J. Vu, X. Meng, F. D. Urnov, E. J. Rebar, P. D. Gregory, H. S. Zhang, R. Jaenisch, Generation of isogenic pluripotent stem cells differing exclusively at two early onset parkinson point mutations. Cell. 146, 318-331 (2011).
109. B. A. Tucker, R. F. Mullins, L. M. Streb, K. Anfnson, M. E. Eyestone, E. Kaalberg, M. J. Riker, A. V. Drack, T. A. Braun, E. M. Stone, Patient-specifc iPSC-derived photoreceptor precursor cells as a means to investigate retinitis pigmentosa. Elife. 2013 (2013), doi:10.7554/eLife.00824.001.
110. P. Douvaras, J. Wang, M. Zimmer, S. Hanchuk, M. A. O'Bara, S. Sadiq, F. J. Sim, J. Goldman, V. Fossati, Efficient generation of myelinating oligodendrocytes from primary progressive multiple sclerosis patients by induced pluripotent stem cells. Stem Cell Reports. 3, 250-259 (2014).
111. J. Basu, J. W. Ludlow, Exosomes for repair, regeneration and rejuvenation. Expert Opin. Biol. Ther. 16, 489-506 (2016).
112. C. J. Alves, L. P. De Santana, A. J. D. Dos Santos, G. P. De Oliveira, T. Duobles, J. M. Scorisa, R. S. Martins, J. R. Maximino, G. Chadi, Early motor and electrophysiological changes in transgenic mouse model of amyotrophic lateral sclerosis and gender differences on clinical outcome. Brain Res. 1394, 90-104 (2011).
113. P. Rabinovich-Toidman, I. Rabinovich-Nikitin, A. Ezra, B. Barbiro, H. Fogel, I. Slutsky, B. Solomon, Mutant SOD1 Increases APP Expression and Phosphorylation in Cellular and Animal Models of ALS. PLoS One. 10, e0143420 (2015).
114. J. Wang, K. Zuzzio, C. L. Walker, Systemic dental pulp stem cell secretome therapy in a mouse model of amyotrophic lateral sclerosis. Brain Sci. 9, 165 (2019).
115. M. A. Lanuza, L. Just-Borràs, E. Hurtado, V. Cilleros-Mañé, M. Tomàs, N. Garcia, J. Tomàs, The Impact of Kinases in Amyotrophic Lateral Sclerosis at the Neuromuscular Synapse: Insights into BDNF/TrkB and PKC Signaling. Cells. 8, 1578 (2019).
116. A. G. Kay, G. Long, G. Tyler, A. Stefan, S. J. Broadfoot, A. M. Piccinini, J. Middleton, O. Kehoe, Mesenchymal Stem Cell-Conditioned Medium Reduces Disease Severity and Immune Responses in Inflammatory Arthritis. Sci. Rep. 7 (2017), doi:10.1038/s41598-017-18144-w.
117. S. Dahbour, F. Jamali, D. Alhattab, A. Al-Radaideh, O. Ababneh, N. Al-Ryalat, M. Al-Bdour, B. Hourani, M. Msallam, M. Rasheed, A. Huneiti, Y. Bahou, E. Tarawneh, A. Awidi, Mesenchymal stem cells and conditioned media in the treatment of multiple sclerosis patients: Clinical, ophthalmological and radiological assessments of safety and efficacy. CNS Neurosci. Ther. 23, 866-874 (2017).

### Example 2

### Autologous Treatment for ALS with Implication for Broad Neuroprotection

### Background

Neuronal cell death causes a plethora of neuro-degenerative diseases, including age-related loss of memory and dementias (such as Alzheimer's Disease), Parkinson's Disease, and Amyotrophic lateral sclerosis (ALS). These diseases have in common an increase in ROS, mitochondrial dysfunctions, protein misfolding and aggregation, neuroinflammation, progressive-catastrophic loss of neurons, and so far, the lack of a cure.

ALS is a progressive neurodegenerative disease with rapid onset of paralysis and respiratory failure, and morbidity within 2 to 5 years of diagnosis. More than 5000 patients in the United States receive a diagnosis of ALS each year [1]. Mutations in superoxide dismutase 1 (SOD1), oxidative damage and protein misfolding are involved in both familial and sporadic ALS [2-7]. The transgenic mouse with human SOD1^{G93A} (B6SJL-TgN[SOD1-G93A]1Gur) is one of the most popular and powerful models to study the ALS as it recapitulates human pathophysiology, such as motor neuron loss, neuromuscular junctions degradation, axonal degeneration and limb paralysis [3,8-11].

Pluripotent stem cells (PSCs), e.g., embryonic stem cells (ESCs), and induced pluripotent stem cells (iPSCs), are used in various biomedical fields due to their capacity of unlimited self-renewal and the ability to differentiate into multiple cell types [12]. iPSCs from patients with ALS (ALS-iPSC) have been established and the derived MNs (ALS-MNs) show pathological characteristics of this disease: diminished neurite length, protein aggregation, and an enhanced apoptosis [13-16] that is reversible by over-expression of Bcl-2 [17].

Considering the limitations of tissue-specific differentiation and oncogenic side-effects of undifferentiated PSCs, stem cell-derived acellular factors might be a safer alternative to cell transplantation[18]. We previously reported the enhancement of old muscle repair by hESC conditioned cell culture medium (CM), and the ability of this embryonic stem cell conditioned medium to absorb/neutralize toxic beta amyloid [19,20]. Several other studies focused on CM from mesenchymal and adipose stem cells [21-24], but none have shown meaningful reversal of the multiple, complex, and mutually enforcing pathologies of ALS.

Here we establish that ALS patient derived iPSC secretome robustly protects their MNs from apoptosis in vitro, prevents MN degeneration in vivo in ALS mice, significantly improves the mitochondrial activity of MNs and the formation and maintenance of neurites and innervation (neuro-muscular junctions), and delays the onset of symptoms and prolongs the lifespan of ALS mice. Biochemical characterization and comparative proteomics suggest that the combined effects of multiple proteins are needed. While ALS iPSC-CM and Cyclosporin A (CsA, an immunosuppressant that blocks mitochondrial pores) both stabilize MN mitochondria, only ALS iPSC-CM, but not CsA, prevents the death of ALS MNs. This work is the first to develop and validate a new feasible biologic for preventing and attenuating neurodegenerative diseases.

### Materials and Methods

### Animals

All animal experimental procedures were performed in accordance with the Guide for Care and Use of Laboratory Animals of the National Institutes of Health, and approved by the Office of Laboratory Animal Care (OLAC), UC Berkeley.

Transgenic mice SOD1^{G93A} (B6SJL-Tg(SOD1*G93A)1Gur/J) were purchased from Jackson Laboratory (No. 002726; Bar Harbor, USA). All mice were randomly assigned to an ALS iPSC-CM group or a dF-CM group.

### Culture of Human Pluripotent Stem Cells and Collection of the Conditioned Medium

Human embryonic stem cells (hESCs, H9, WiCell Institute) and human induced pluripotent stem cells (hiPSC, WTC-11) were obtained through the UC Berkeley Cell Culture Facility. The SOD1-mutated familial ALS-iPSCs, CS07 and CS53, were obtained from Cedars-Sinai medical center (Los Angeles, California) (Table 5).

TABLE 5
The list of amyotrophic lateral sclerosis cell lines.

| ALS-IPSC Line | Parent Cell | | | Age at Sample |
|---|---|---|---|---|
| Name | Type | Mutation | Sex | Collection |
| CS07iALS- | Fibroblast | SOD1 - A4V | Female | 40 |
| SOD1A4Vnxx | | (A5V) | | |
| CS53iALS- | Peripheral | SOD1 - A4V | Male | 35 |
| SOD1A4VNTnxx | blood | (A5V) | | |
| | mononuclear | | | |
| | cell | | | |

All human pluripotent stem cells (hPSCs) were maintained on plates coated with Vitronectin (Life technologies) and cultured in Essential-8 medium (Life technologies) at 37° C. in a 5% CO₂ atmosphere. All hPSCs were passaged every 7 days by 0.5 mM EDTA (Life technologies). For the collection of conditioned medium, hiPSCs were cultured to 80% to 90% confluence. Then, the cells were washed twice (5 min each wash) with Opti-MEM (ThermoFisher, Carlsbad, CA) and cultured in Opti-MEM for 24 hours and the conditioned medium was collected: hESC-CM, hiPSC-CM, and ALS-CM, as published [20]. The collected CMs were centrifuged at 10,000 g for 30 minutes at 4° C., and filtered through 0.22 um syringe filter (Millipore-Sigma). CMs from the same groups were pooled (7 to 10 dishes) and stored in aliquots at −80° C. before use. For the CM from differentiated cells, fibroblasts were derived by first differentiating hESCs and hiPSCs into embryoid bodies (EB)s in 100-mm Petri dishes in Essential 6 (Life technologies) at 37° C. in a 5% CO₂ for 5 days, and then into fibroblasts for 7 days and two passages on 0.1% gelatin coated tissue culture dishes (Falcon) in Dulbecco's Modified Eagle's Medium (DMEM), 10% fetal calf serum (FCS), 1% (v/v) non-essential amino acid, and 1% (v/v) penicillin-streptomycin. CM from fibroblasts was collected and used identically to the PSC CM.

### Culture of Human Fibroblast Cell Line

Human lung fibroblast cells, IMR-90 (CCL-186, ATCC), were maintained in DMEM (Dulbecco's Modified Eagle Medium) supplemented with 10% fetal calf serum (FCS, Hyclone) containing 1% penicillin-streptomycin (Invitrogen) and maintained in a humid atmosphere at 37° C. containing 5% CO₂. Cells were grown to 70% confluence and subcultured at 1:5 split when passaging.

### Motor Neuron Precursor (MNP) and Motor Neuron (MN) Differentiation

The process of MNP and MN differentiation was as published [25]. Briefly, hPSCs were detached by 0.5 mM EDTA and split 1:10 on Matrigel-coated plates (1:50 dilution). After one day, the Essential 8 was changed to neural inducing medium containing neural basal medium (NBM) plus 3 μM CHIR99021 (Tocris), 2 μM LDN-193189 (Tocris) and 3 μM SB431542 (Stemgent). The NBM is composed of DMEM/F12, Neurobasal medium (FisherScientific) at 1:1, N2, B27, 0.1 mM ascorbic acid (Millipore-Sigma), 1× Glutamax and 1× penicillin/streptomycin (all from Invitrogen). The inducing medium was maintained for 5 days and changed every other day. On day 5, the cultured cells were split at 1:6 with Accutase and cultured in NBM containing 3 μM CHIR99021, 2 μM SB431542, 0.1 μM retinoic acid (RA, Stemgent) and 0.5 μM purmorphamine (Pur, Stemgent). After 7 days, MNPs were generated and expanded with MNP medium, which consisted of NBM supplemented with 3 μM CHIR99021, 2 μM SB431542, 0.1 μM RA, 0.5 μM Pur and 0.5 mM valproic acid (VPA, Stemgent). MNPs were passaged every 7 days by exposure to Accutase and cryopreserved in liquid nitrogen in DMEM/F12, 10% Knockout serum replacement and 10% DMSO. To induce MN differentiation, MNPs were dissociated with Accutase and cultured in suspension in the above NBM with 1 μM RA and 0.1 μM Pur. The medium was changed every other day. After 7 days, MNPs differentiated into MNs. The MNs were then dissociated with Accutase into single cells and plated on PLO/laminin-coated plates. The MNs were cultured into mature neurons in NBM plus 1 μM RA, 0.1 μM Compound E (Calbiochem), 10 ng/ml Insulin-like growth factor 1, 10 ng/ml Glial cell-derived neurotrophic factor (ThermoFisher), and 10 ng/ml Brain-derived neurotrophic factor (ThermoFisher).

Isolation of Heparin-Binding Proteins (HBPs) from Conditioned Medium

The HBPs of hESC-CM were prepared as published [20]. Briefly, Heparin-Agarose Type I Beads (H 6508, Sigma Aldrich) were washed with molecular grade water and incubated with CM from the various cells for 2 hours with agitation at 4° C., then samples were centrifuged at 10,000 g for 20 minutes. The collected heparin beads were washed two times for 10 minutes at 4° C. in washing buffer (1 ml phosphate-buffered saline (PBS)+0.05% Tween-20). The HBPs were eluted twice for 15 minutes at 4° C. in 400 μl of elution buffer (0.01M Tris-HCl pH 7.5+1.5M NaCl+0.1% BSA). The collected proteins were re-equilibrated to the cell culture medium by dialysis for 2 hours at 4° C. in 500 ml McCoy's 5A Medium (Gibco) followed by overnight dialysis at 4° C. in 200 ml Opti-MEM (Gibco). The eluted heparin bound proteins were diluted to 800 μl with Opti-MEM and stored at −80° C. before use.

Isolation of Exosomes from Conditioned Medium

For the isolation of exosomes, 100 ml samples of hESC-CM were centrifuged at 2,000 g for 10 minutes to remove cells and debris, followed by centrifugation at 10,000 g for 30 minutes at 4° C. to remove microvesicles [26]. The supernatant was filtered through 0.22 μm filters (Millipore-Sigma) and then the filtered CM was sedimented by ultra-centrifugation at 120,000 g for 90 minutes in an i70 rotor (Beckman Coulter, Brea, CA, US). The pellet was washed once with PBS and re-sedimented at 120,000 g, 4° C. for 90 minutes. Finally, the pellet was resuspended in 200 μl PBS and stored at −80 C for further use.

### Treatment of Cyclosporine (CsA)

To investigate the effect of CsA (11-011-00, Tocris), MNs were cultured with 10 μM CsA for 3 or 5 days. Then, cell viability, apoptosis, qPCR, and Elisa were performed.

### Cell Viability Assay

After H₂O₂ and/or CM or CM-derived factors treatment, the number of viable cells was determined by a resorufin based assay using the commercially available CellTiter-Blue® cell viability assay (G8080, Promega), as per the manufacturer's protocol. MNs were cultured in a 96-well plate at a cell density of 2×10⁴ and treated with H₂O₂ and/or CMs for 24 hours before performing the viability assay. A SpectraMax iD3 Multi-Mode Microplate Reader (Molecular Devices) was used for measuring fluorescence intensity (560/590 nm).

### Antioxidant Assay

The antioxidant assay was performed according to the manufacturer's instructions (BioAssay systems, DTAC-100). Briefly, each sample (20 μL) was mixed with kit reagent (100 μL). Then the mixed sample was incubated 10 min at room temperature. The color intensity at 570 nm is measured by a SpectraMax iD3 Multi-Mode Microplate Reader. The values ranging from 0-1000 μM Trolox were used as a reference.

### Real-Dime Polymerase Chain Reaction (PCR)

Total RNA was extracted using the RNeasy mini kit (Qiagen), and the SuperScript III First-Strand Synthesis System (Invitrogen) was used to synthesize cDNA according to the manufacturer's instructions. Real-time PCR was performed on a Bio-Rad iQ5 real-time PCR machine. The primers used for PCR are listed in Table 6.

TABLE 6
Primer sequences for Real
Time Polymerase Chain Reaction

| | Gene | | SEQ |
|---|---|---|---|
| Group | name | Sequence | ID NO |
| Pluri- | hOCT4_F | GGGCTCTCCCATGCATTCAAAC | 13 |
| potency | hOCT4_R | CACCTTCCCTCCAACCAGTTGC | 14 |
| | hNANOG_F | TGGGATTTACAGGCGTGAGCCAC | 15 |
| | hNANOG_R | AAGCAAAGCCTCCCAATCCCAAAC | 16 |
| Motor | OLIG2_F | GTT CTC CCC TGA GGC TTT | 17 |
| neuron | | TC | |
| precursor | OLIG2_R | AGA AAA AGG TCA TCG GGC | 18 |
| | | TC | |
| Motor | HB9_F | GTC CAC CGC GGG CAT GAT | 19 |
| neuron | | CC | |
| | HB9_R | TCT TCA CCT GGG TCT CGG | 20 |
| | | TGA GC | |
| | CHAT_F | GGA GGC GTG GAG CTC AGC | 21 |
| | | GAC ACC | |
| | CHAT_R | CGG GGA GCT CGC TGA CGG | 22 |
| | | AGT CTG | |
| | ACTB_F | TGA AGT GTG ACG TGG ACA | 23 |
| | | TC | |
| | ACTB_R | GGA GGA GCA ATG ATC TTG | 24 |
| | | AT | |
| Inflam- | IFNB1_F | TGTCGCCTACTACCTGTTGTGC | 25 |
| mation | IFNB1_R | AACTGCAACCTTTCGAAGCC | 26 |
| | TNF_F | TCTCTCAGCTCCACGCCATT | 27 |
| | TNF_R | CCCAGGCAGTCAGATCATCTTC | 28 |
| House- | HPRT_F | TCAGGCAGTATATCCAAAGATGGT | 29 |
| keeping | HPRT_R | AGTCTGGCTTATATCCAACACTTCG | 30 |
| gene | | | |

### Immunofluorescence Staining

Cells were fixed in 4% paraformaldehyde for 30 minutes, permeabilized with 0.25% Triton X-100 and blocked with 5% FCS in PBS for 1 hour. The fixed cells were incubated overnight at 4 C in PBS+1% FCS with antibodies against Mouse anti-OCT4 (1:500, ab 18976, Abcam), Rabbit anti-SOX2 (1:500, MA516399, ThermoFisher), Rabbit anti-TUJ1 (1:500, MAB1195, R&D system), Rabbit anti-OLIG2 (1:500, NBP128667, Novus), Rabbit anti-HB9 (1:500, ABN 174, Millipore-Sigma), CHAT (1:500, AB 144P, Abcam), and Cleaved caspase 3 (1:500, 9669S, Cell Signaling Technology) followed by incubation with secondary antibodies: FITC-conjugated anti-mouse IgG (1:1000, A21202, Life Technologies), FITC-conjugated anti-rabbit IgG (1:1000, A11034, Life Technologies), Cy3-conjugated anti-mouse IgG (1:1000, A11003, Life Technologies), and Cy3-conjugated anti-rabbit IgG (1:1000, A11035, Life Technologies). The treated cells were covered with slow-fade anti-fade with DAPI (Life Technologies) for nuclear staining and covered with a glass coverslip. Images were captured with a fluorescence microscope (DM5000B, Leica).

### Apoptosis Assay

The apoptosis assay of fibroblasts was by Annexin V-CF Blue/7-AAD Apoptosis Detection Kit (ab214663, Abcam) according to the manufacturer's protocol. Briefly, cells were detached using 0.05% trypsin and washed twice with PBS. Then, samples were resuspended in 1× annexin-binding buffer and incubated with 5 μL Annexin V-FITC and 5 μL 7-amino-actinomycin D (7AAD) for 15 min at 37° C., avoiding light. Finally, the stained samples were analyzed on a Guava Easycyte Flowcytometer (Millipore-Sigma) at an excitation wavelength of 488 nm and emission filters of 525 and 625 nm. For MNs, the apoptotic assay was conducted on two different markers, FITC-conjugated-annexinV (ab201540) or Cleaved caspase 3 (9669S). For annexinV detection, MNs in 96-well plates were washed twice with PBS. Then, 100 μL Annexin V binding buffer was added with 5 μL Annexin V-FITC for 5 min incubation at 37° C., avoiding light. The stained plate was analyzed on a SpectraMax iD3 Multi-Mode Microplate Reader (490/525 nm). For the Cleaved caspase 3, the immunofluorescence protocol was used.

### High-Content Analysis

For differentiation efficiency, analysis neurite outgrowth analysis, and apoptosis analysis, plates were imaged using the high-content imaging system, ImageXpress Micro (IXM, Molecular Devices); a set of 5×5 or 7×7 fields was collected from each well using the 10× or 20× objective. Data were further analyzed in MetaXpress 6 software (Molecular Devices).

### Analysis of Neurite Length

During differentiation into MNs, cells were sampled at 5 day intervals between 20 and 60 days. MNs were cultured in 8-well chamber slides (LabTek II CC2 coated), stained with Tuj1 and imaged by IXM. MetaXpress 6 software was used to analyze the mean process length as a measure of the total outgrowth divided by the number of processes of the cell. Analysis was performed on a total of 4-10 fields and at least 1000 cells per group from three independent experiments. For the neurite size comparison, the data were normalized by the values at day 20; shrinkage of neurites over time was determined as net changes from neurite sizes on days 20 and 25.

### Enzyme-Linked Immunosorbent Assay (ELISA)

ELISA was analyzed for IP-10 protein (DIP100, R&D system) and the sample dilution was performed according to the manufacturer's protocol (1:20-50). The stained plate was analyzed on a SpectraMax iD3 Multi-Mode Microplate Reader.

### Antibody Array

CMs from pluripotent stem cells or differentiated cells were analyzed on a human L1000 antibody capture array (AAH-BLG-1000, Raybiotech), processed according to the manufacturer's protocol. The array slides were imaged by a Molecular Devices 4000b scanner and data were calculated by Genepix. After normalization by dF-CM, differently changed proteins over 2-fold up- or 0.5-fold down-regulated were selected.

### Epigenetic Inhibitors Screen

For the collection of CM, separate cultures of hiPSCs were set up for 3 days with one of the following small molecules: 5 μM Nanomycin A (A8191, Apexbio), 10 μM LSD1 inhibitor (489476, Millipore-Sigma), 5 μM 5-Azacytidine (C832A53, Thomas Scientific), and 10 μM GSK126 (67-905, Tocris). After 3 days of treatment, CM was collected (without the inhibitors) using the same protocol as described above.

### Measurement of Mitochondrial Stress

Motor neurons were detached with Accutase, washed with PBS and incubated for 10 min with 5 μM Mitosox Red (M36008, Thermo Fisher) at 37° C. in the dark. After three washes in warm PBS, stained MNs were resuspended in warm FACS buffer (1% FCS in PBS) and analyzed by a Guava Easycyte Flow cytometer (620/52 nm).

### Bioinformatics Analysis

The gene ID of differently regulated proteins was performed using DAVID Bioinformatics Resources (version 6.8, https://david.ncifcrf.gov), as well as the GO (Gene Ontology) analysis of the differential proteins based on the biological process (BP), molecular function (MF) and cellular component (CC), and Kyoto Encyclopedia of Genes and Genomes (KEGG) pathway. Heat maps were performed with ClustVis [27]. Violin plots were constructed with the Python seaborn package and normalized with a standard min-max scaler.

### Conditioned Medium Treatment and Behavior Test

The CM of ALS iPSCs (CS07 and CS053, Cedar-Sinai) or CM of fibroblasts was injected subcutaneously at the neck at a volume of 200 μl every other day as described [28].

For behavior test, we monitored the weight, neurological scoring, and hanging time every other day. First, all mice were weighed. Then, we assayed them by neurological scoring, as published [29] with criteria as follows:
Score of 0: While mouse is suspended by the tail, the hindlimbs are fully extended away from the lateral midline and keep the status for two seconds.
Score of 1: Partial collapse of leg extension or trembling of hind legs during tail suspension. This stage is considered an onset of symptoms.
Score of 2: While mouse is suspended by the tail, the hindlimbs not extending much or completely collapsed. During walking of 90 cm, toes curling or dragging of leg parts is observed.
Score of 3: Rigid paralysis or minimal joint movement is observed. Hindlimbs not being used much for forwarding motion. We considered this stage the end-stage.

All mice were euthanized at a score of 3. All information was recorded for each mouse every other day until the end-stage (a score of 3).

For hanging test, mice were placed on a grid and then turned upside down. Then, the latency to fall was measured. The latency time measurements began from the point when the mouse was hanging free on the grid and ended with the animal falling to the cage underneath the grid.

### Survival

For survival assessment, 11 female (8 ALS iPSC-CM group and 3 dF-CM group) and 11 male (8 ALS iPSC-CM group and 3 dF-CM group) SOD1^{G93A} mice were used. It was considered that animals reached the end point of the disease when they showed signs of paralysis, with a score of 3, above.

Serum Collection from Blood

Mice were anesthetized with isoflurane and blood samples were collected by heart puncture [30]. The tubes with whole blood were allowed to clot for 30 min at room temperature. Then, the clot was removed by centrifuge at 2000 g for 10 min. The supernatant was collected as serum and stored at −80° C. before use. For western blot, each serum was diluted 1:20 with Laemmli sample buffer and heated for 10 min at 95° C.

### Tissue Collection

When mice showed the first signs of paralysis in hind limbs or 120 days, euthanasia was proceeded with the guidelines of UC Berkeley's OLAC administration. For histological comparison, some ALS mice were euthanized at 120 days. Spinal cords and muscles (Gastrocnemius and Tibialis anterior) were isolated. Then, spinal cords were fixed with 4% paraformaldehyde (PFA) overnight at 4° C. and muscle tissues were fixed for 30 min at room temperature. Then, all tissues were transferred to 30% sucrose solution overnight and embedded in tissue-tek optimal cutting temperature (OCT, Sakura Finetek, The Netherlands) and snap frozen in isopentane cooled to −70° C. with dry ice. Then, all samples were stored at −80° C. before use.

For histological analysis, all OCT-embedded tissues were cryosectioned by a Cryostar NX50 (ThermoFisher). Muscle tissues were sectioned to 20 μm thickness while spinal cords were obtained at 10 μm.

### Muscle Weight

To compare the wet muscle weight of the GA and TA in all groups, mice were euthanized and muscle tissues were isolated, and both left and right muscles were weighed on a scale. For the comparison, all weight recorded was normalized by their body weight.

### Cresyl Violet Staining and Quantification

Mounted frozen sections were dried overnight at 37° C. and placed slides directly into 1:1 alcohol/chloroform overnight, then rehydrate through 100% alcohol to distilled water. Next, sections were stained in 0.1% cresyl violet solution for 5-10 minutes at 37° C., rinsed with distilled water, dehydrated with ethanol and cleared in xylenes, and mounted in Permount (ThermoFisher). Stained sections were imaged at 5× and 10× magnification. For quantification, five sections were selected randomly and total number of MNs of the ventral spinal cord was counted using unbiased methods, as described [31].

### Neuromuscular Junction Staining and Quantification

Neuromuscular junction (NMJ) analysis was performed as previously described [32]. Briefly, dried sections were permeabilized with 1% Triton X-100 and blocked with 5% FCS in PBS for 1 hour. The fixed cells were incubated for 2 hours at room temperature in PBS+1% FCS with antibodies, Alexa 488-conjugated neurofilament (1:500, 8024, Cell Signaling Technology), Alexa 488-conjugated synaptophysin (1:500, MAB5258A4, Millipore-Sigma), and Alexa 555-conjugated a-bungarotoxin (1:500, B35451, Life Technologies). The stained slides were covered with slow-fade anti-fade with DAPI (Life Technologies) for nuclear staining and covered with a glass coverslip. Images were captured with a fluorescence microscope (DM5000B, Leica). For quantification, five sections were selected randomly and counted. Intact NMJs (yellow) were identified as those that exhibited an overlay of a-bungarotoxin (red) and neurofilament (green). Denervated NMJs were defined by labeling with only a-bungarotoxin (red). Percent intact NMJs was calculated as the number of intact NMJs (yellow)/total number of motor end plates (yellow+red) as described [32].

### Statistical Analysis

All statistical analyses were performed using GraphPad Prism software version 9 (GraphPad software Inc). All values are expressed as means±SEM for independent experiments, or SD for replicates. To determine the significance of differences among groups, comparisons were made using Student's t-test. Survival and onset data was analyzed with Kaplan-Meier curves and log rank test. The P<0.05 was considered significant.

### Results

### Autologous ALS IPSC-CM is Neuroprotective for Motor Neurons of ALS Patients

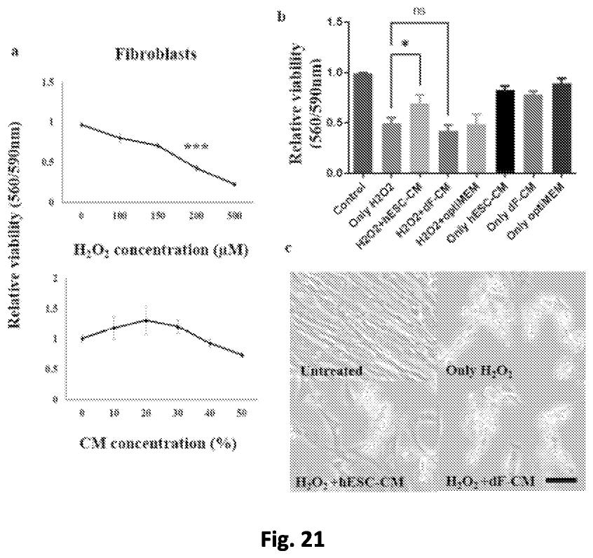

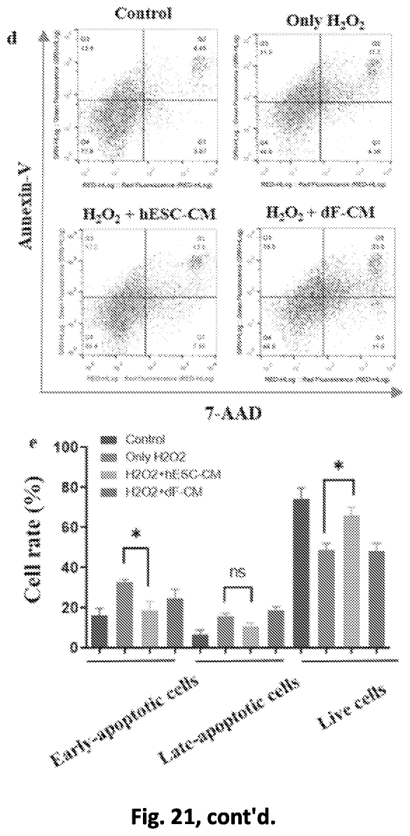

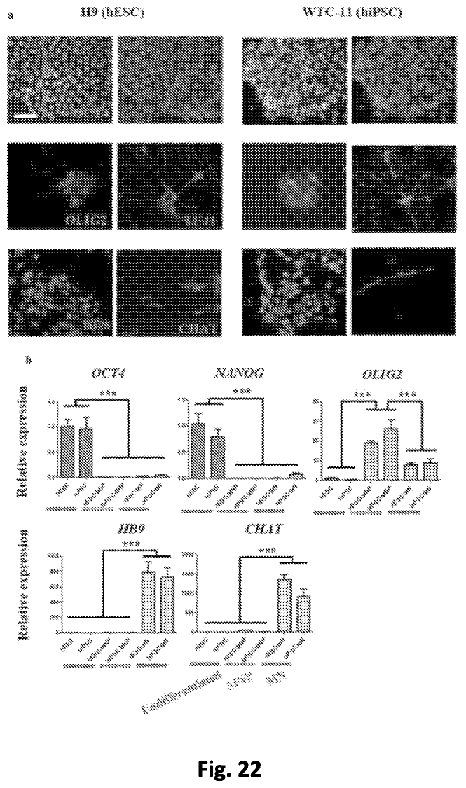

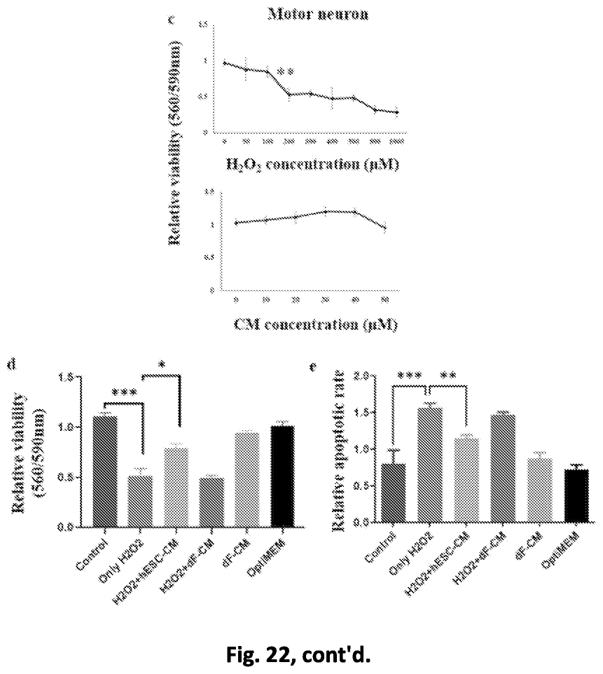

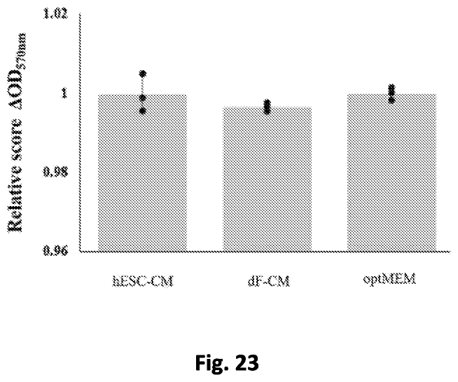

hESC conditioned medium (CM) was previously shown to enhance proliferation of myoblasts and rat neural precursor cells [19,20], but the effects on cell viability were less studied. We decided to explore a protective role of hESC-CM when cells experience oxidative damage, which is physiologically and clinically relevant in a number of human diseases [33,34]. hESC-CM protected human fibroblasts and human MNs from H₂O₂ caused cell death (FIGS. 21 and 22; and Table 7). Additional antioxidant activity of hESC-CM was tested and not found (FIG. 23), suggesting it did not simply neutralize ROS to preserve cell viability. Control CM, produced by hESCs differentiated into fibroblasts (dF-CM) did not have such neuroprotective properties.

TABLE 7
The efficiency of motor neuron differentiation
from normal pluripotent stem cells.

| H9 | | | | | |
|---|---|---|---|---|---|
| WTC11 | | | WTC11 | | |
| # of | # of HB9 | | # of | # of HB9 | |
| DAPI | positive | % | DAPI | positive | % |
| 11270 | 10612 | 94.16 | 15642 | 14543 | 92.97 |
| 11130 | 10290 | 92.45 | 11818 | 10573 | 89.47 |
| 9654 | 8982 | 93.04 | 6492 | 6039 | 93.02 |
| | 93.21 ± 0.86 | | 91.82 ± 2.03 | | |

Encouraged by the neuroprotective effects of hESC-CM on human MNs, we tested the hypothesis that iPSC-CM is also neuroprotective and will promote the viability of ALS patient derived MNs. If true, this would suggest a novel completely autologous approach to treat neurodegenerative diseases. We studied two ALS patient derived iPSC lines (CS53-male and CS07-female) with the most common mutation in the SOD1 gene, A4V (Table 8).

TABLE 8
The efficiency of motor neuron differentiation
from ALS patient hiPSCs.

| CS07 | | | CS53 | | |
|---|---|---|---|---|---|
| | # of | | | # of | |
| # of | double | | # of | double | |
| DAPI | positive | % | DAPI | positive | % |
| 18909 | 17273 | 91.35 | 20094 | 18149 | 90.32 |
| 6316 | 5776 | 91.45 | 11520 | 10430 | 90.54 |
| 5687 | 5103 | 89.73 | 11703 | 10256 | 87.64 |
| | 90.84 ± 0.96 | | 89.49 ± 1.61 | | |

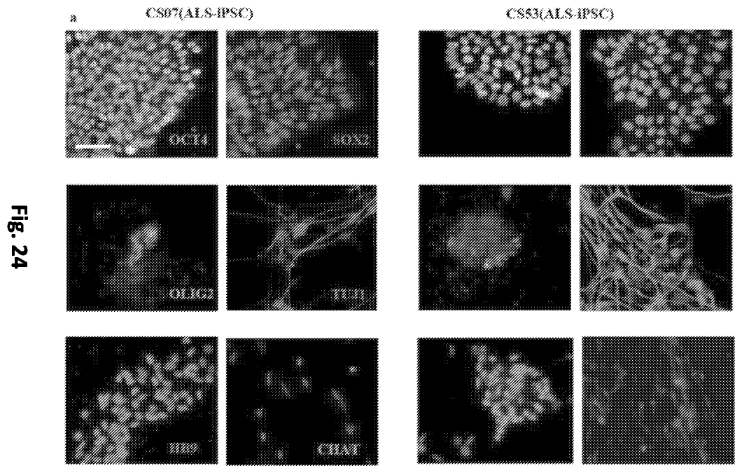

These ALS-iPSC lines were confirmed for their markers of pluripotency and then differentiated into MN precursors (MNPs) and MNs, in parallel with normal, wild-type SOD1 human iPSCs (WTC11), as confirmed by the immuno-detection of OLIG2, Tuj1, and HB9 (FIG. 24, panel a) and by qPCR on OCT4 and NANOG, OLIG22, HB9, and CHAT (FIG. 24, panel b). Interestingly, the WTC11 and ALS iPSC cells did not differ in the expression of pluripotency markers or morphology at iPSC stage, or in their MN markers when differentiated, FIGS. 22 and 24. Additionally, there was no difference in the derivation efficiency of WTC11-MN and ALS-MN (Tables 7 and 8).

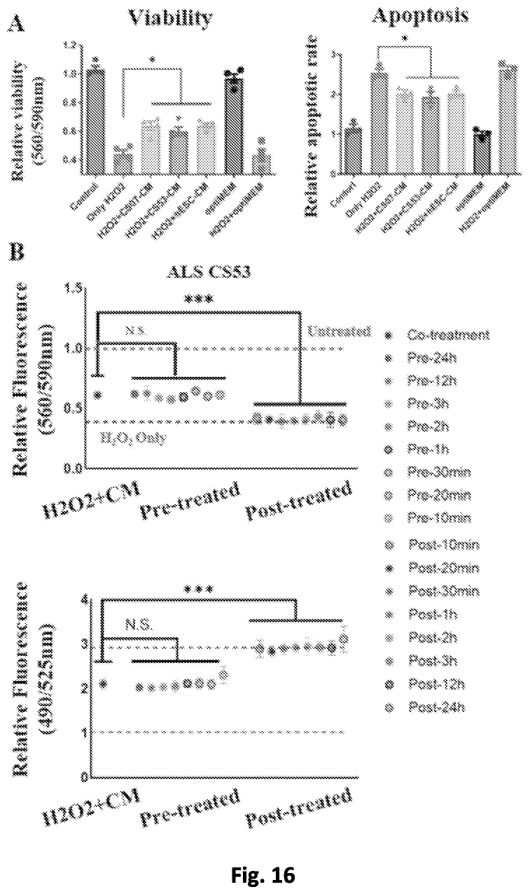

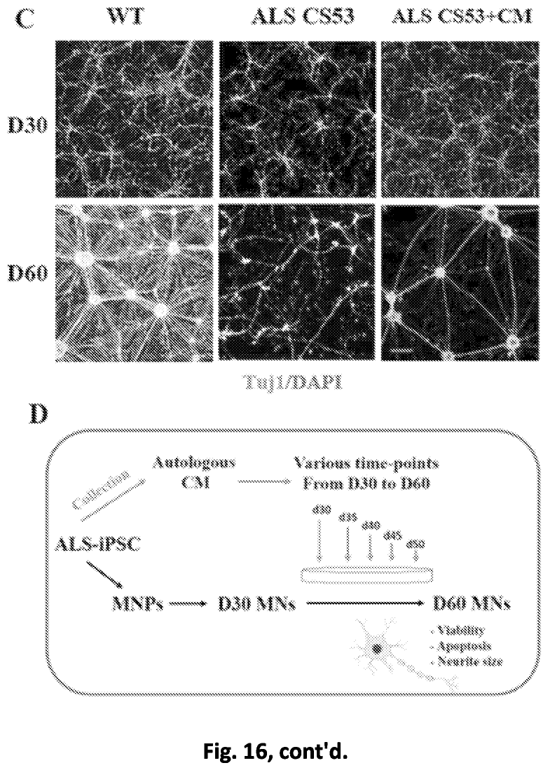

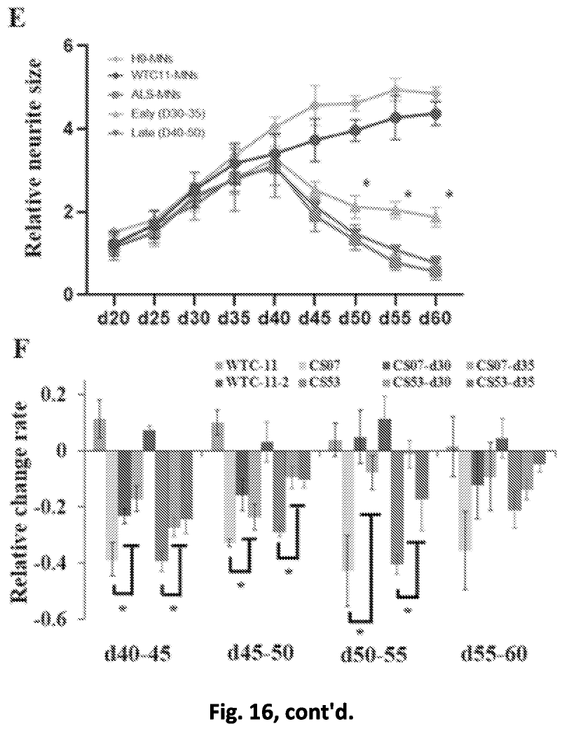

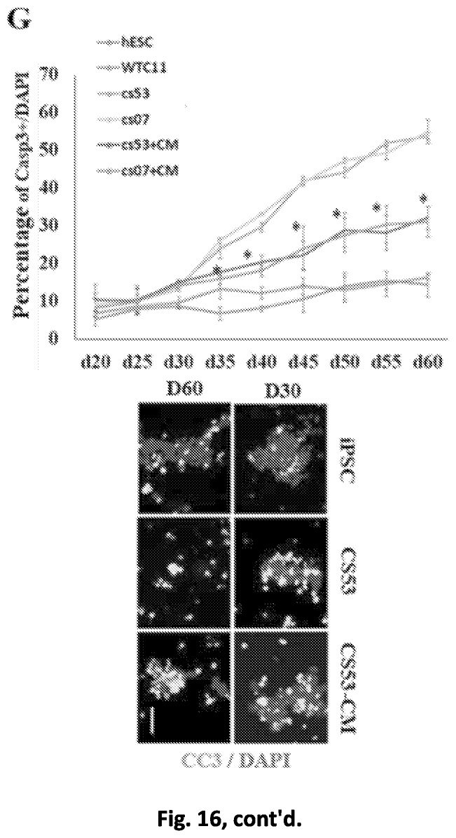

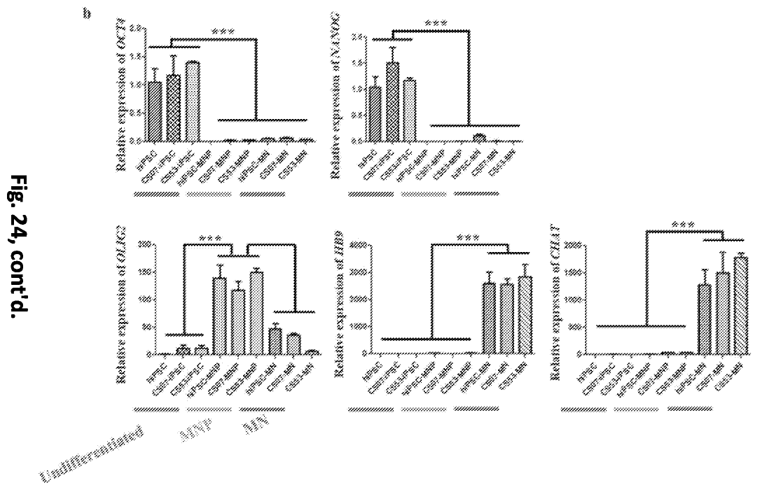

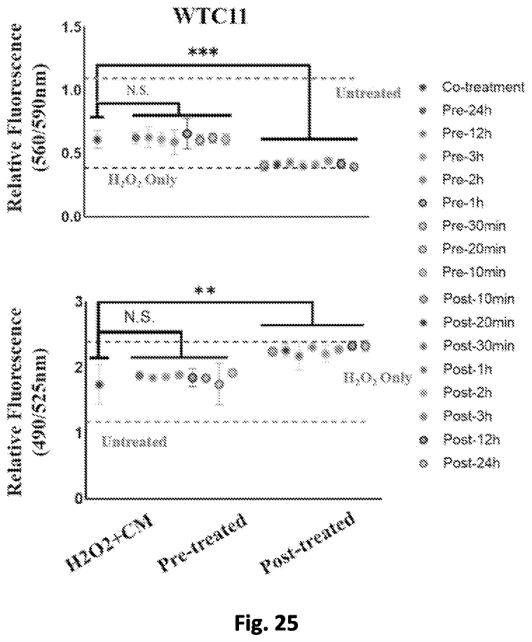

When exposed to H₂O₂, MNs had diminished viability and increased apoptosis, and interestingly all tested PSC conditioned media, hESC-CM, WT iPSC-CM and most importantly the ALS patient derived iPSC-CM, had neuroprotective effects in this experimental set-up (FIG. 16, panel a). We further explored the timing of this neuroprotective effect of healthy human iPSC-CM and ALS patient iPSC-CM on MNs by either pre-treating the cells with these CMs for variable time points before H₂O₂(Pre-treatment) or alternatively, by treating the cells with H₂O₂ first for variable time points before adding the CMs (Post-treatment). The viability and apoptotic assays demonstrated that iPSC-CM is critically needed to be present at least 10 minutes before H₂O₂ is added to protect the MNs from the cytotoxic effects, whereas the addition of iPSC-CMs after H₂O₂(10 minutes to 24 hours) does not confer this neuroprotection, FIG. 16, panel b, and FIG. 25.

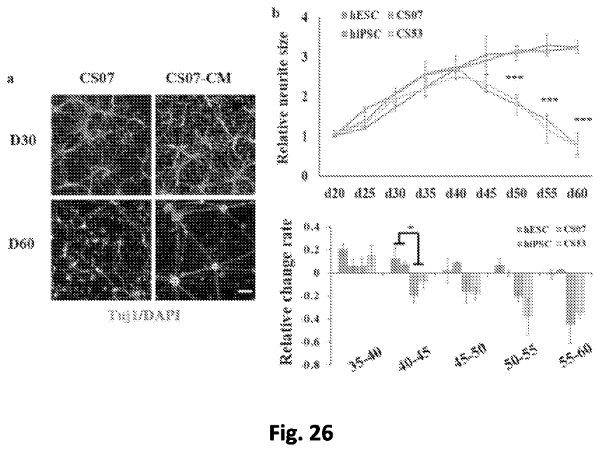

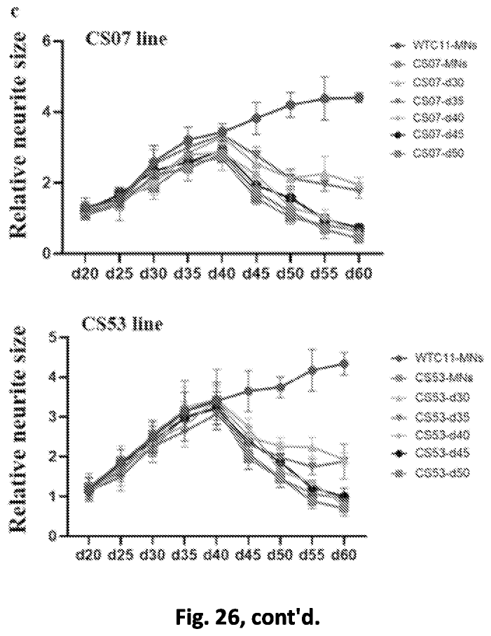

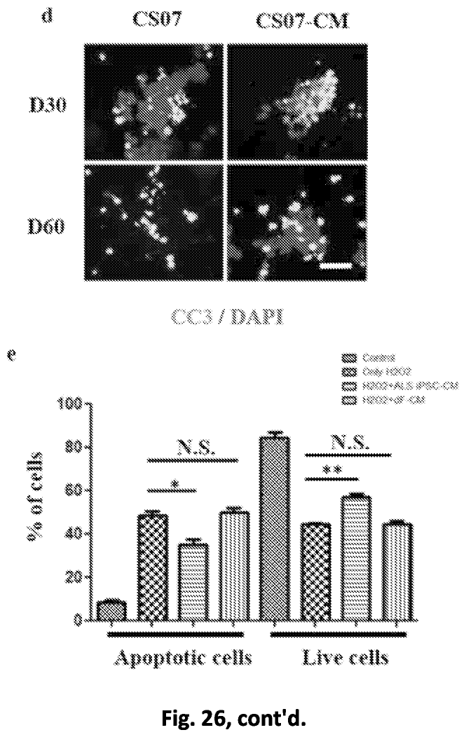

ALS-MNs are known to show a decrease in the size of their neurites as compared to wild type MNs [14], and thus we studied the effect of ALS iPSC-CM on this physiologically important parameter in the primary ALS-MN lines (CS53 and CS07) that were treated with their autologous ALS iPSC-CMs. As shown in FIG. 16, panel c, FIG. 26, panels a and b, neurite size increased gradually for 40 days of differentiation of iPSCs into MNs with no difference between the wild type and ALS cells. However, at 45 days of differentiation and all subsequent time points, the neurite size markedly shrunk in the ALS cohort as compared to the wild type cells (FIG. 16, panel c and FIG. 26, panel b). Neurite length fluctuation suggested that 40 to 45 days represent a key transition time-point of regulation of neurite outgrowth where shrinkage becomes prominent in ALS cells.

Considering that pathological changes in the MNs start to manifest at ˜40 days of their differentiation from the ALS hiPSCs (FIG. 26, panels a and b), the CM additions to the culture medium were performed as a time-course, on days 30, 35, 40 and 45 (FIGS. 16, and 26, panel d). The assays of neurite size showed that the maintenance of neurites improved in the groups treated with ALS iPSC-CM starting at either day 30 or day 35, (FIG. 16, panel e, and FIG. 26, panel c). Moreover, the rate of neurite size degeneration was significantly reduced by ALS iPSC-CM added on days 30 and at 35 in both ALS-MN lines (FIG. 16, panels e and f). However, when added at the later time point, e.g., at 40 days, ALS iPSC-CM was not able to rescue the neurite size of ALS-MNs (FIG. 16, panel e and FIG. 26, panel c).

We also analyzed the relative numbers of apoptotic cells in these ALS-MNs (CS53 and CS07). There was a slight upward trend in the number of cleaved caspase3 (CC3) positive cells between the wild type and ALS-MN lines up to day 30, but from day 35 onward the number of CC3 positive cells started to dramatically increase in ALS-MN cultures as compared to the healthy MN cultures (FIG. 16, panel g, and FIG. 26, panel d). Notably, the rate of ALS-MNs apoptosis was significantly attenuated by the autologous ALS iPSC-CM, for both ALS-MN cell lines (FIG. 16, panel g, and FIG. 26, panel e).

These data reveal that ALS patient iPSC-CM has multi-functional neuro-protective effects on their ALS MNs, improving cell viability and the maintenance of neurites.

### Neuroprotection, Improved Neuro-Muscular Function, and Delayed Morbidity of ALS Mice Bearing the Human Disease SOD1^{G93A}.

To determine whether the CMs from ALS patient-iPSCs could have the same neuro-protective effects in vivo, we used an ALS mouse model. Transgenic mice engineered to carry the mutant human SOD1 (SOD1^{G93A}) gene are used extensively to study human ALS [29,35,36]. These mice display progressive degeneration of MNs and the phenotypes of ALS and thus, we decided to use this model for assessing the effects of ALS iPSC secretome in vivo. The first symptoms occur in these mice at 80-90 days of age and morbidity takes place at 128±9 days [37], and so we studied from the asymptomatic stage (68 days) through the entire progression of the disease to the morbidity endpoint.

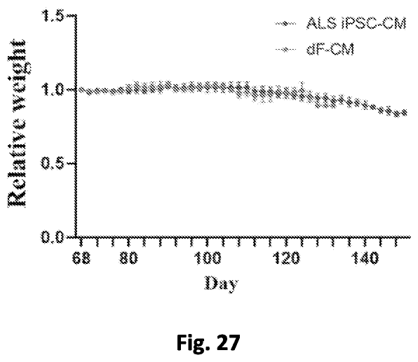

Twelve ALS mice were studied for the effects of ALS iPSC-CM (CS07 and CS53) and 6 ALS mice were studied in parallel, using the negative control, dF-CM. Age matched males and females were identically treated with either ALS iPSC-CM or dF-CM. Throughout the study, including up to the endpoint, there was no significant difference in animal weights between ALS iPSC-CM treated and the negative control, dF-CM treated mice (FIG. 27).

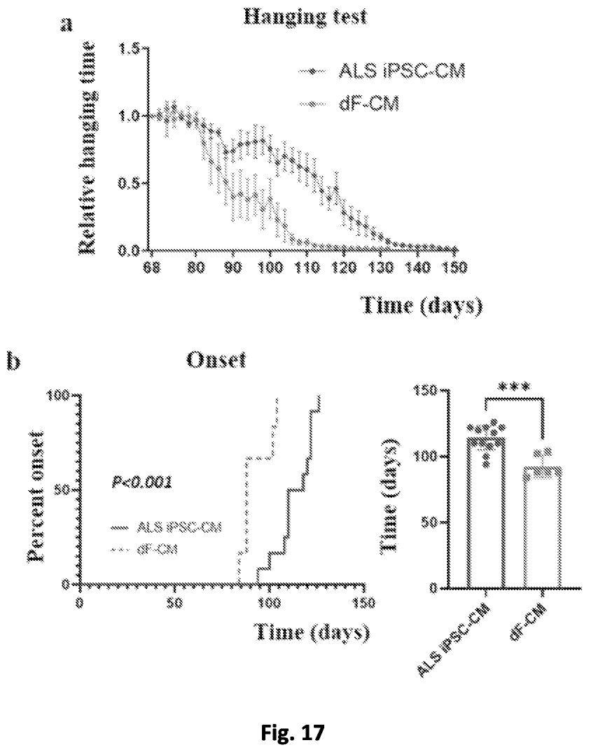

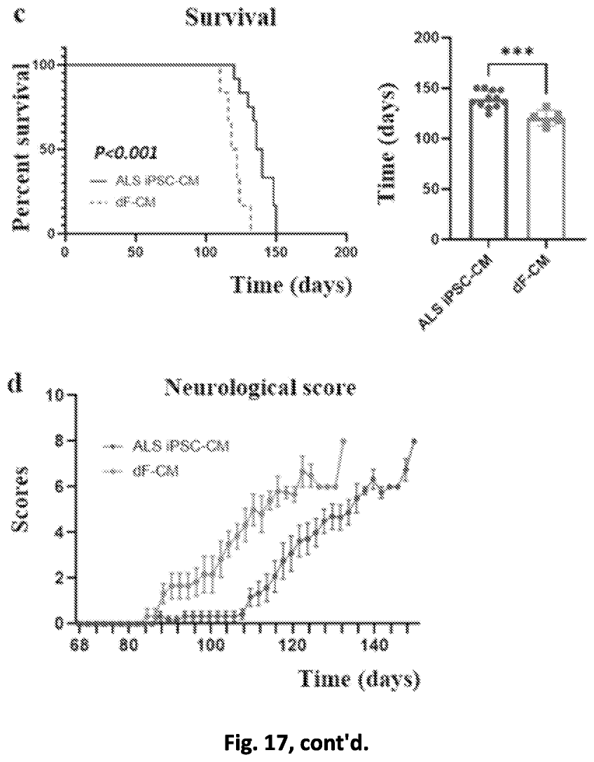

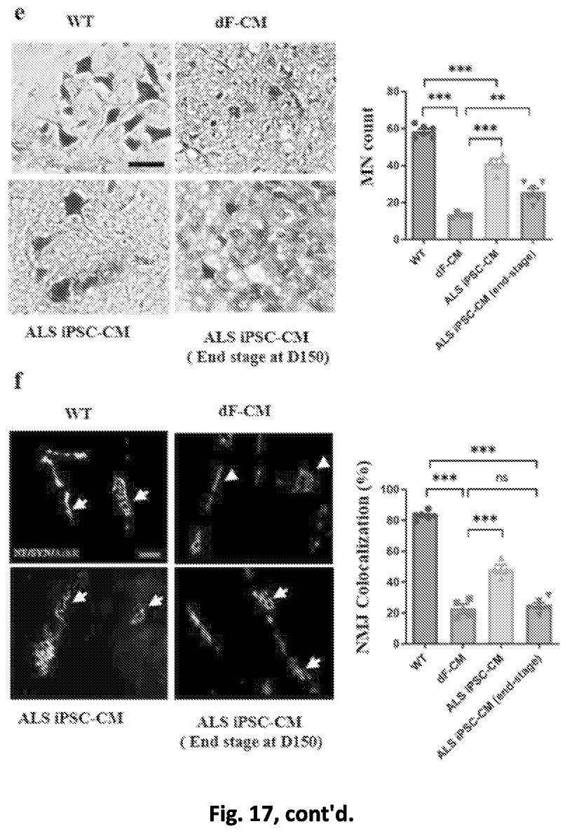

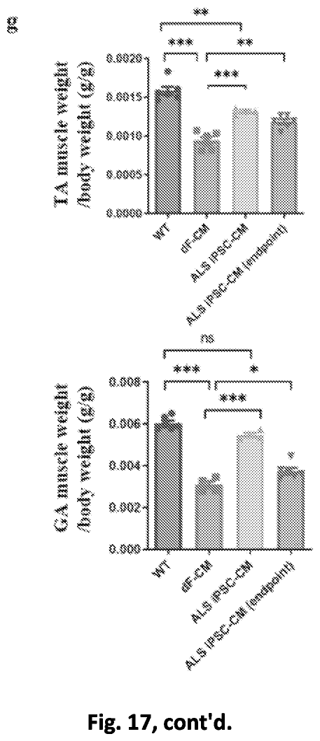

The control dF-CM treated mice rapidly deteriorated in their neuro-muscular function, which we measured through neurological scoring [29] and four-limb hanging tests [38]. Remarkably, CM of ALS patient iPSCs rescued the agility, coordination, and overall muscle function of the SOD1 mutant mice, delayed the onset of pathological symptoms (FIG. 17, panels a and b), delayed morbidity (FIG. 17, panel c) and improved the neurological scores of these ALS model animals (FIG. 17, panel d). In agreement with the neuro-muscular improvements, survival was significantly prolonged in the ALS iPSC-CM group as compared to the dF-CM group (141.5±3.26 days vs. 120.3±3.07 days, P<0.001) (FIG. 17, panel c).

To investigate the effect of hiPSC-CM on the lumbar spinal cord and neuromuscular junction, these structures were analyzed postmortem in 120 days old mice. Compared to the C57.B6 wild type animals, the total number of cresyl violet stained MNs in the ventral spinal cord was decreased in the control SOD1^{G93A} mice that were treated with dF-CM (58.5±3.33 vs. 13.5±1.25, P<0.001), indicating progression of the disease (FIG. 17, panel e). In addition, many cresyl violet stained MNs showed shrunken morphology with no neurites in the SOD1^{G93A} dF-CM group as compared to the C57.B6 (FIG. 17, panel e). However, the number of MNs was significantly increased in the SOD1^{G93A} mice treated with ALS iPSC-CM, as compared to the dF-CM group (41.25±5.12 vs. 13.5±1.25, P<0.001), and was still greater than in the negative control at the end-stage (at 150 days) 25.50±5.32 vs. 13.5±1.25, P<0.05, FIG. 17, panel e. These results demonstrate that the treatment of ALS model mice with ALS human iPSC-CM prevents deterioration of spinal MNs.

NMJ defects contribute to the decline in muscle mass and health [38], and NMJ alterations are a feature of neurological diseases [39-41]. Thus, we investigated NMJ integrity and muscle innervation, using the specific markers acetylcholine receptor (AchR), neurofilament and synaptophysin. Our results show that the number of NMJs was significantly reduced in the dF-CM group, as compared to the C57.B6 group at day 120 (FIG. 17, panel f), while the ALS iPSC-CM group had significantly more intact NMJs than the dF-CM group (48.46±5.43 vs. 22.87±6.12, P<0.001). As expected, at end-stage, there was no difference between dF-CM and the ALS iPSC-CM groups (FIG. 17, panel f). These findings suggest that the treatment of SOD1G93A mice with ALS iPSC-CM preserves muscle innervation longer, which is consistent with the improved 4-limb handing test performance and better neurological scores.

Better NMJs suggested a reduction in muscle atrophy, so we determined the weights of Gastrocnemius (GA) and Tibialis anterior (TA) of the studied groups of mice. The TA and GA mass was normalized by the animal body weight. As expected, there was a significant decrease in the average weights of GA and TA in the negative control, dF-CM, compared to the wild type C57.B6 group. Encouragingly, the weights of GA and TA were significantly increased in the ALS iPSC-CM group, as compared to the dF-CM group (FIG. 17, panel g).

Summarily, these data demonstrate that ALS iPSC-CM significantly delays the onset of the paralysis and functional decline in the SOD1^{G93A} mice, extends their lifespan and protects MNs, NMJs and muscle mass in this animal model of human progressive neurodegenerative disease. Such profound multiparametric positive effects on ALS, averting and delaying all key manifestations of this deadly disease, have not been previously reported with other tested methods.

### Mechanisms, Relation to Pluripotency, Sub-Cellular Details and Improvement Over CsA

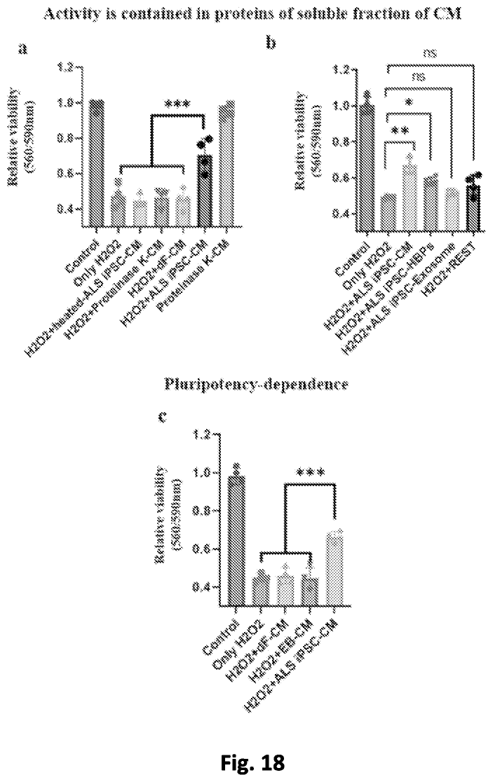

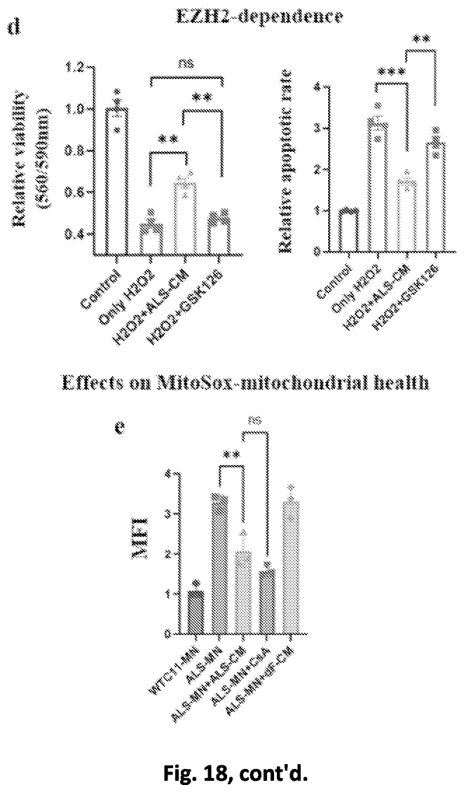

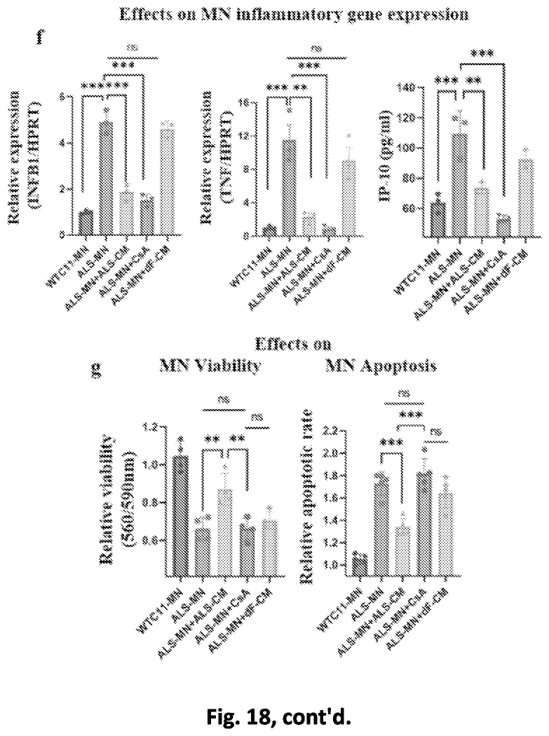

To understand the mechanisms by which the iPSC secretome (CS07) is neuroprotective in ALS, we performed biochemical characterizations. First, we investigated whether the effect was produced by proteins. Heat inactivation and Proteinase K treatments abrogated the positive effects of the iPSC secretome, demonstrating that neuroprotective activity is contained in the protein fraction (FIG. 18, panel a).

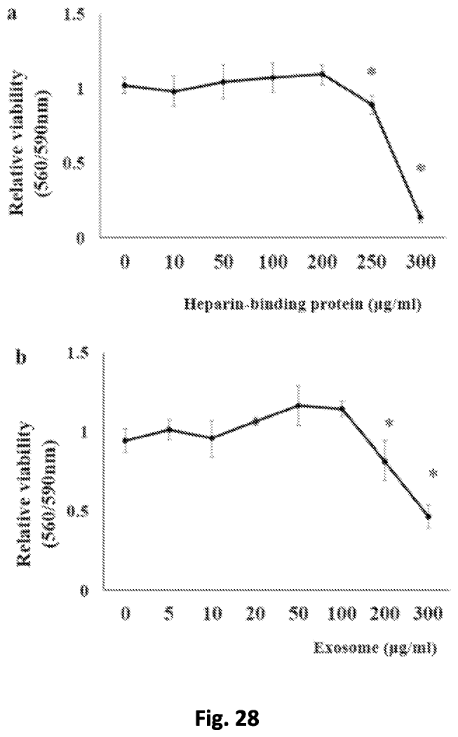

Several reports have shown that the exosomes from stem cell-CM has a protective effect on cellular damage/stress [26,42]. Our previous studies documented that heparin binding proteins from hESC-CM had promoted proliferation of muscle and neural cells [19,20]. Accordingly, we examined whether the neuroprotective activity is contained in the heparin-bound (HB) versus unbound soluble fractions and exosome fraction. First we determined the maximum amount of HB fraction tolerated by cells, and the HB fraction became cytotoxic at 300 μg/ml (FIG. 28, panel a). Exosomes, purified from hESC CM by ultracentrifugation, [26], negatively affected cell viability at 200 μg/ml (FIG. 28, panel b). Based on these data, 200 μg/ml HBPs and 100 μg/ml exosome preparations were chosen for treating MNs. There was an increase in viability of human MNs that were co-treated with H₂O₂ and HB fraction, as compared to H₂O₂ alone (0.58±0.02 vs. 0.49±0.01, P<0.05); however, the efficiency of that neuroprotection was less than that displayed by the unfractionated PSC-CM (0.66±0.04 vs. 0.58±0.02, P<0.05) (FIG. 18, panel b). There was no improvement in viability or decrease in apoptosis of MNs that were co-treated with H₂O₂ and the exosome fraction (FIG. 18, panel b). The soluble fraction flow through, that did not contain exosomes or HB proteins, had a slight but not statistically significant trend of neuroprotection (FIG. 18, panel b). These data suggest that neuro-protective activity is contained in multiple fractions of PSC-CM, and mostly in the soluble fractions (heparin bound and unbound).

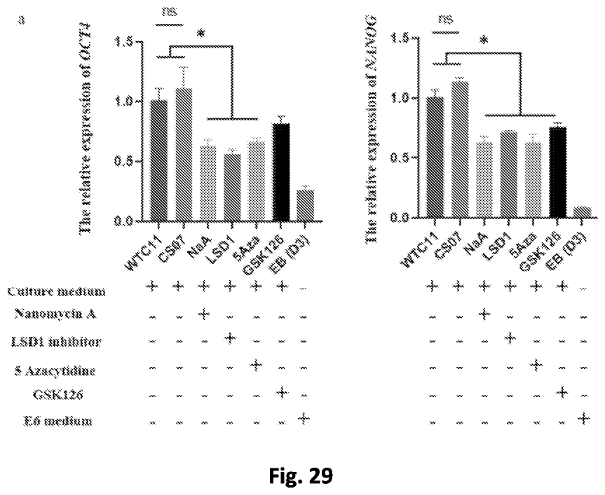

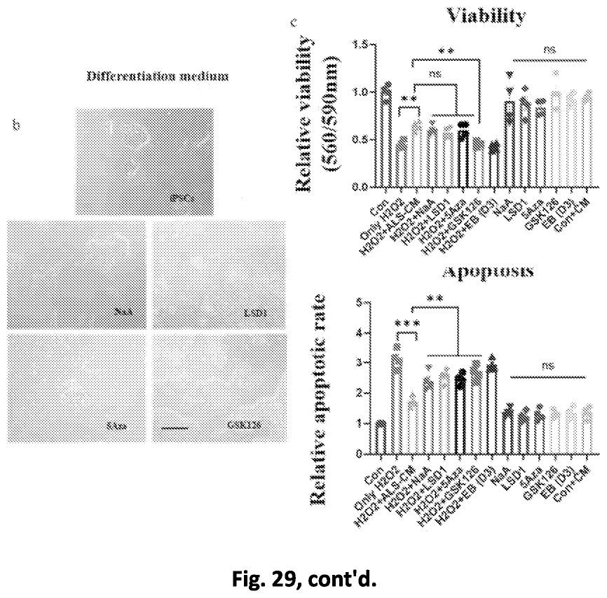

Based on the absence of neuroprotective activity in the differentiated progeny of iPSCs, we decided to confirm and mechanistically extrapolate the relationship between the protective effects and pluripotency. As shown in FIG. 18, panel c, differentiation of PSCs into embryoid bodies negated the neuroprotective activity, expanding the data on PSC-derived fibroblasts and confirming that a state of pluripotency is required for the neuroprotective activity. To follow on the requirement for pluripotency, we employed a screen with inhibitors of pluripotency that modify the epigenetic states of iPSCs. We treated separate cultures of ALS iPSCs with the inhibitors: Nanomycin A (NaA), LSD1 inhibitor (LSD1), 5 Azacytidine (5Aza), or GSK126 (FIG. 18, panel d, and FIG. 29). After 3 days in culture with each of these small molecules, the expression of pluripotency markers decreased (FIG. 29, panel a). However, pluripotent colony-like morphologies were maintained, and OCT4 and NANOG expression was higher in each single inhibitor culture, as compared to the longer and more profound differentiation of iPSCs into EBs (FIG. 29, panels a and b). To determine the effects of epigenetic modifiers on the neuroprotective properties, we studied the viability and apoptosis of MNPs exposed to H₂O₂ that were pre-treated with CM from ALS iPSC exposed to the epigenetic inhibitors. Interestingly, treatment of ALS iPSCs with EZH2 inhibitor, GSK126, significantly reduced the neuroprotective activity of the ALS iPSC-CM (FIG. 18, panel d), while other inhibitors showing no difference or non-statistical trend (FIG. 29, panel c). This suggests the possibility that an EZH2-dependent pathway regulated by GSK126 is important for the neuroprotective activity of the ALS iPSC secretome.

To understand the sub-cellular effects of the ALS iPCS secretome on MN, we assayed mitochondrial health, which is known to diminish in ALS MNs, and generally in neurons of patients with neurological diseases [43,44]. Specifically, we performed the mitoSox red mitochondrial ROS analysis on ALS and healthy MNs, and on the ALS MNs that were cultured with the ALS iPSC-CM as compared to the dF-CM, FIG. 18, panel e. As expected, ALS MNs had higher mean fluorescence intensity (MFI) of mitoSox red as compared to the healthy MNs, and the positive control, treatment with immunosuppressive cyclosporin A (CsA) that blocks mitochondrial pores, reduced the MFI (FIG. 18, panel e). Notably, autologous ALS iPSC-CM significantly reduced the MFI of mitoSox red, as compared to untreated or dF-CM treated MN, to a degree that was statistically indistinguishable from CsA (FIG. 18, panel e).

We also explored the relationship of ALS iPSC-CM to neuroinflammation, e.g., the shared phenotype of many neurological disorders, including ALS [45-47]. ALS iPSC-CM significantly diminished the expression of pro-inflammatory INFB1, TNF-α, and IP-10 in ALS MNs, and these effects were similar in the CsA-treated ALS MNs (FIG. 18, panel f).

Since CsA and ALS iPSC-CM both improved mitochondria and reduced expression of INFB1, TNF, and IP-10, we compared them side-by-side for neuroprotection: in diminishing apoptosis and improving the viability of human ALS MNs. Interestingly, only ALS iPSC-CM, but not CsA, reduced apoptosis and increased viability of the SOD1 mutant ALS MNs that were derived from patients with this disease (FIG. 18, panel g).

These results establish that ALS iPSC-CM activity is mediated by multiple soluble secreted proteins (not exosomes), which likely act together since fractionation results in diminished activity of every fraction, as compared to the unfractionated CM. The ALS iPSC-CM activity is dependent on the state of pluripotency and on an EZH2 dependent process; it improves mitochondrial health and reduces neuro-inflammatory gene expression. These data also show that while CsA blocks mitochondrial pores and both CsA and ALS iPSC-CM stabilize mitochondria, there are additional effects of ALS iPSC-CM on averting the death of ALS MNs.

### Identification of a Neuro-Protective Protein Interactome

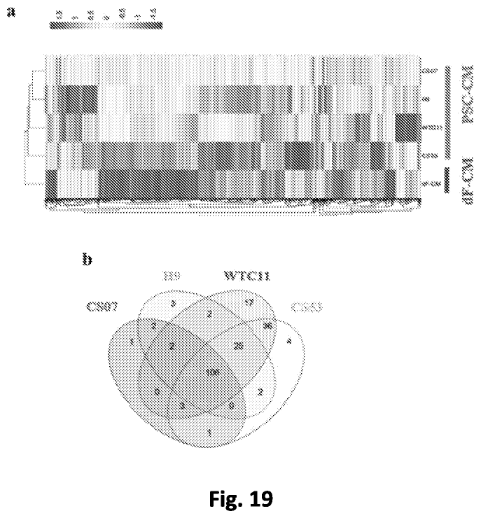

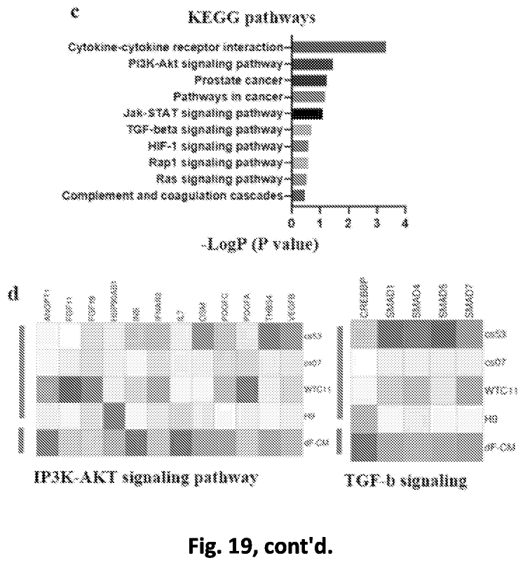

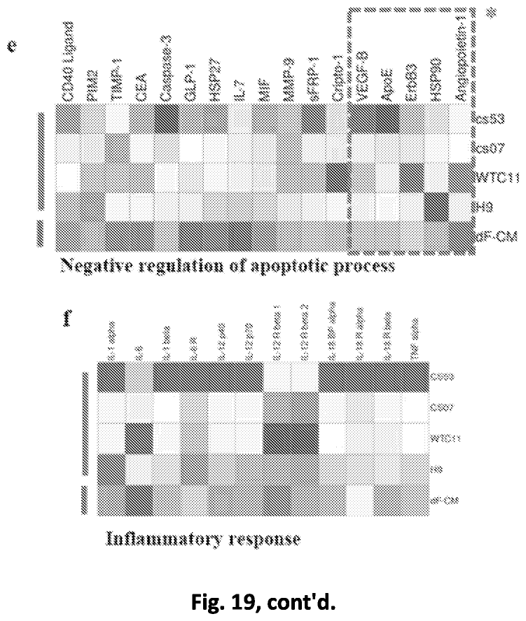

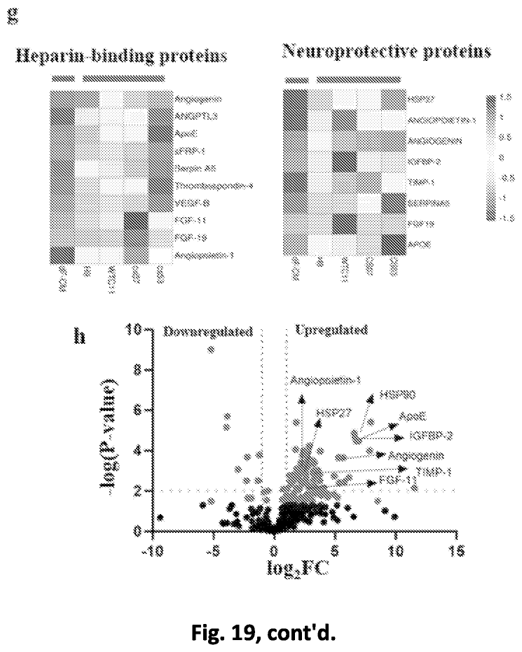

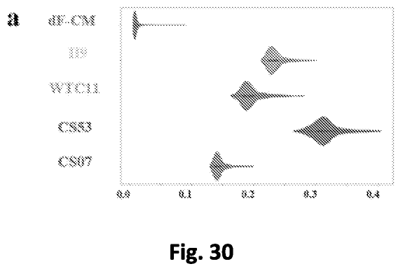

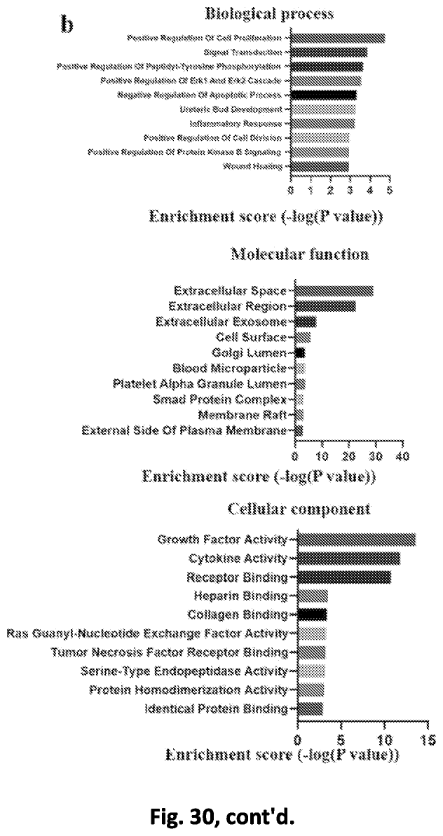

To identify candidate neuroprotective proteins, we performed comparative proteomic antibody capture arrays that detect 1000 factors, comparing hESC-CM, WT iPSC-CM and ALS iPCS-CM (CS07 and CS53) that all have neuroprotective activity, with each other and with the dF-CMs that lack neuroprotective activity (FIG. 19, panel a). This screen identified 106 candidates (Venn diagram, FIG. 19, panel b), that were up-regulated in all PSC-CM groups, as compared to the dF-CM (>2-fold change with p<0.05). The violin plot showed that the proteome patterns of hESC-CM, WT iPSC-CM and ALS iPSC-CM were significantly different from those of dF-CM (FIG. 30, panel a). The list of KEGG pathways was constructed through grouping the proteins by their key functions (FIG. 19, panel c). PSC-CMs and dF-CMs significantly differed in the levels of regulatory proteins that participate in canonical morphogenic signaling pathways, including PI3K-Akt, Jak-STAT, BMP/TGF-β, and Ras, all of which were shown to be important for development, viability and/or maintenance of neurons (FIG. 19, panel d) [48].

Additional protein characterizations as per the Biological Process (BP), Molecular Function (MF), and Cellular Component (CC) by DAVID are shown in FIG. 30, panel b. In agreement with our functional data, the BP group had 170 GO terms, including positive regulation of cell proliferation, signal transduction, positive regulation of peptidyl-tyrosine phosphorylation, negative regulation of apoptosis, control of wound healing, and beta-amyloid clearance; the latter is highly consistent with our previously reported [20] neuroprotection from ectopic beta-amyloid (FIG. 19, panel e, and FIG. 30, panel b).

One of the most important causes of MN degradation includes the inflammatory response [45-47]. The proteomic screening showed that several proteins and cytokines known to induce inflammation, including IL-1, IL-6, IL-12, and TNF-α, were reduced in PSC-CM compared to dF-CM, (FIG. 19, panel f).

Interestingly, in concert with the above fractionation experiments and our previous findings on heparin-binding activity of pro-regenerative hESC-CM [19,20], we identified several heparin-binding proteins that were elevated in the PSC-CM cohorts as compared to the dF-CMs, including angiogenin (ANG), Angiopoietin-1(ANG-1), angiopoietin like 3, apolipoprotein E (ApoE), secreted frizzled related protein 1, serpin family A member 5, thrombospondin 4, vascular endothelial growth factor B, fibroblast growth factor 11 (FGF11), and fibroblast growth factor 19 (FGF19), FIG. 19, panel g. Most of these heparin-affinity factors promote cell proliferation and attenuate cell death, in agreement with our current data on neuroprotection [49-53].

Among proteins that do not bind to heparin, but were identified in the neuroprotective PSC secretome (FIG. 19, panel g), TIMP1 has shown protective effects against not only traumatic and ischemic brain injury but also HIV-1-induced neuronal apoptosis [53,54]. IGFBP-2 is involved in regulation of cell proliferation and, has an anti-apoptotic effect via modulation of caspase-3 [55,56]. IGFBP-2 may have a role in neuroprotection from the hypoxic-ischemic injury and protects neurons from beta-amyloid-induced toxicity [57,58]. HSP27 has an intrinsic responsibility in neuro-protection by increasing the cell viability and decreasing apoptotic signaling [59-61]. Comparing PSC-CM and dF-CM through volcano plots, the identity of proteins which may play important roles in various aspects of neuroprotection were determined (FIG. 19, panel h).

The comprehensive comparative proteomics suggests that activities of multiple ALS iPSC secreted proteins have the capacity, in concert, to avert the degeneration and death of ALS MNs: some proteins inhibit apoptosis by independent from each other mechanisms, while others reduce protein aggregation, reduce ROS damage, improve post ROS repair, and/or support neuronal cell fate.

### Discussion

This work demonstrates that proteins secreted by PSCs are neuroprotective and importantly, that ALS patient derived iPSCs can be used to combat their disease. The main cause of damage and death of neurons in ALS and other neurological diseases is oxidative stress, [4,5,62,63]. Thus, we first established that hESC-CM was able to protect fibroblasts and MNs from oxidative stress induced apoptosis. This neuroprotective activity was then confirmed in ALS patient hiPSCs-CM. H₂O₂ is an extreme neurotoxin, hence it is important that in various screens and all independent experiments PSC-CM consistently enhanced the viability of MN and diminished their apoptosis by ˜20% in the presence of H₂O₂. Next, we established that proteins that are secreted by the wt and ALS hiPSCs promote cell viability, formation and stability of neurites, and mitochondrial health of SOD1 mutant human ALS MNs. In vivo, human ALS iPSC-CM delayed the onset of symptom and extended the lifespan of SOD1 mutant ALS transgenic mice, through better maintenance of MNs and NMJs. ALS iPSC-CM could slow down, but not overcome the progression of ALS, which might be improved when the defined proteins are determined and tested in future work.

The autologous patient's iPSC secretome avoids the problems of cell transplantations: incomplete differentiation, the danger of transplanting cancer-causing cells and difficulties of iPSCs maturation [64-66]. Moreover, the autologous approach using the patient's own hiPSCs will minimize the variability of heterologous hiPSCs and an immune response to heterologous proteins. Reprogramming of disease-specific differentiated cells into iPSCs often results in normal pluripotency, even when there are disease-associated mutations, and in our work, ALS-iPSCs were typical pluripotent stem cells, based on their markers and the efficiency of MN differentiation [67-69].

Mechanistically, our results suggest that that the activity is protease sensitive and that a combination of proteins from different biochemical fractions are needed for the neuroprotection (FIG. 18, panel a). Based on our comparative proteomics, the candidate proteins that were enriched in the PSC-CM as compared to the non-active dF-CM, are either secreted or can be secreted when cells are damaged, and/or via exosomes or microvesicles, [70,71], Table 9. Because the neuroprotective activity of the hiPSC secretome is diminished when either exosomal or soluble fractions are removed, both fractions likely have proteins that confer neuroprotection through their interactome.

TABLE 9
The 106 PSC-CM protein candidates and their secretory capacity.

| Name | Secreted | Non-secreted |
|---|---|---|
| Aldolase C | ∘ | |
| Alpha Lactalbumin | ∘ | |
| ANGPTL3 | ∘ | |
| ApoA2 | ∘ | |
| ApoE | ∘ | |
| BCAM | ∘ | |
| MUC1 | ∘ | |
| MUC16 | ∘ | |
| Caspase-3 | ∘ | |
| Cathepsin B | ∘ | |
| CBP | | ∘ |
| TfR | ∘ | |
| CEA | ∘ | |
| Ceruloplasmin | ∘ | |
| Chemerin | ∘ | |
| CHI3L1 | ∘ | |
| CK-MB | ∘ | |
| Clusterin | ∘ | |
| C2 | ∘ | |
| C5a | ∘ | |
| Corticosteroid-binding globulin | ∘ | |
| C-Peptide | ∘ | |
| Troponin T | ∘ | |
| Cytokeratin 19 | ∘ | |
| BNP | ∘ | |
| ACTH | ∘ | |
| Exostosin-like 2 | ∘ | |
| Ferritin | ∘ | |
| Fibrinopeptide A | ∘ | |
| FSH | ∘ | |
| GLP-1 | ∘ | |
| GMNN | | ∘ |
| Hemopexin | ∘ | |
| HSP27 | ∘ | |
| HSP90 | ∘ | |
| IL-34 | ∘ | |
| Kallikrein 2 | ∘ | |
| Kallikrein 10 | ∘ | |
| Lyn | ∘ | |
| NPTXR | | ∘ |
| P-Cadherin | ∘ | |
| PIM2 | | ∘ |
| PPARg2 | | ∘ |
| PR Isoform B | ∘ | |
| PSA-free | ∘ | |
| PTPRD | | ∘ |
| Ret | | ∘ |
| Serpin A5 | ∘ | |
| SHBG | ∘ | |
| SOX2 | | ∘ |
| Angiogenin | ∘ | |
| Angiopoietin-1 | ∘ | |
| CCR5 | | ∘ |
| CD40 Ligand | ∘ | |
| Chordin-Like-1 | ∘ | |
| Cripto-1 | ∘ | |
| CXCR6 | | ∘ |
| EG-VEGF | ∘ | |
| ErbB3 | ∘ | |
| FGF-11 | ∘ | |
| FGF-19 | ∘ | |
| GDF-9 | ∘ | |
| GDF-11 | ∘ | |
| Granzyme A | ∘ | |
| GRO | ∘ | |
| HCR | ∘ | |
| NRG1 Isoform GGF2 | ∘ | |
| IFN-alpha/beta R2 | ∘ | |
| IGFBP-2 | ∘ | |
| IL-38 | ∘ | |
| IL-7 | ∘ | |
| IL-17 RB | ∘ | |
| IL-17 RD | ∘ | |
| IL-21 | ∘ | |
| IL-23 R | ∘ | |
| IL-26 | ∘ | |
| IL-29 | ∘ | |
| Insulysin | | ∘ |
| Kremen-2 | | ∘ |
| MFRP | | ∘ |
| MIF | ∘ | |
| MIP 2 | ∘ | |
| MMP-9 | ∘ | |
| MMP-16 | ∘ | |
| MMP-25 | ∘ | |
| NRG1 Isoform GGF2 | ∘ | |
| Orexin-A | ∘ | |
| Oncostatin M | ∘ | |
| OX40 Ligand | ∘ | |
| PDGF-AA | ∘ | |
| PDGF-C | ∘ | |
| ROBO4 | | ∘ |
| sFRP-1 | ∘ | |
| SIGIRR | | ∘ |
| SMAD1 | ∘ | |
| SMAD4 | ∘ | |
| SMAD5 | ∘ | |
| SMAD7 | ∘ | |
| Spinesin | ∘ | |
| TACI | ∘ | |
| Thrombospondin-4 | ∘ | |
| TIMP-1 | ∘ | |
| TLR1 | | ∘ |
| TRADD | | ∘ |
| TWEAK | ∘ | |
| VEGF-B | ∘ | |

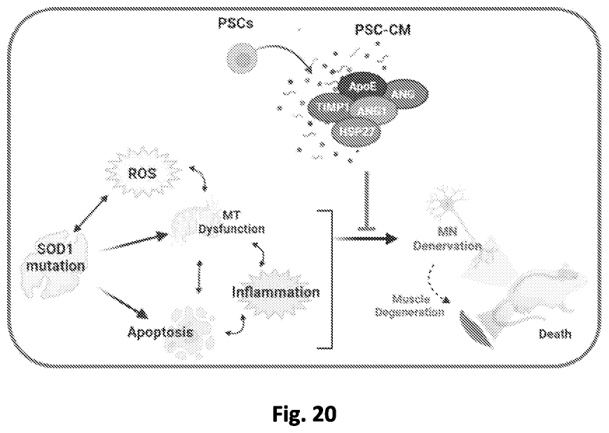

Uncovering the specific interactions of specific proteins that are necessary and sufficient is clearly a subject of future work. At the same time, several neuroprotective candidates seem to be particularly promising, and FIG. 20 summarizes their literature-predicted activities. TIMP1, ANG, ANG1, HSP27, and APOE were enriched in PSC-CM as compared to the negative control, dF-CM, and each of these proteins has known neuroprotective effects [52-54,72-80].

TIMP1 protects neurons against not only traumatic and ischemic brain injury but also HIV-1-induced neuronal apoptosis [53,54]. Angiogenic ANG [81,82] is necessary for success in maturation of neurons, including MNs in the spinal cord and brain [83,84], protects MNs from hypoxia, and contributes to enhanced viability of MNs and longer lifespan of ALS mice [74,75]. ANG 1 is another angiogenic factor in the secretomes of stem cells [82,85,86] that has anti-inflammatory [87] and neuroprotective anti-apoptotic effects [76-79]. HSP27 is antioxidant and inhibitor of procaspase-3 activation [88-90], and these activities are being explored in Alzheimer's [61,91] and ALS models [72,80]. ApoE reduces oxidative stress and contributes to beta-amyloid disposal [52,73]. Moreover, all these proteins interact at the levels of apoptotic, TGF-P, AKT, and ERK signaling pathways [74-79,90], thus functional synergy is possible.

Several clinical trials suggest positive effects of mesenchymal and adipose stem cell secretomes [21,92-95], but interestingly, we show that pluripotency is required for the positive effects and that differentiation of PSCs into fibroblasts or even into EBs abrogates neuroprotection. Epigenetic modification is one of major regulators of pluripotency [96]. Our study demonstrates an EZH2-mediated mechanism of the neuroprotective effect. EZH2 is a key part of the Polycomb Repressive Complex, histone methyltransferase [97]. The expression of EZH2 is not only required for pluripotency [98,99] but influences differentiation into various cell fates, including neurons [100-102]. And EZH2 depletion was shown to induce cell cycle arrest, decrease cell proliferation and induce apoptosis [103,104]. These reports are in concert with our findings that GSK126 treatment of iPSCs reduces the neuroprotective anti-apoptotic activity of their secretome. Interestingly, some of the proteins identified in our comparative proteomics crosstalk with EZH2. For example, HSP90 is involved in EZH2 stability, levels and function [105,106]; ANG-1 and HSP27 are regulated by EZH2 and their expression is decreased upon EZH2 inhibition [107,108]. The conclusion that state of pluripotency is needed for the neuroprotective effects of PSC secretome fits well with the notion that pluripotent stem cells maintain low levels of oxidative stress via oxidation-reduction (redox) signaling, as compared to their differentiated progeny [109-112].

At the cellular levels, ALS-iPSC CM improved the mitochondrial stability of MNs and reduced the expression of inflammatory genes. Recently suggested as an ALS treatment, CsA [47] had similar effects on mitochondrial and MN expressed inflammatory genes, but in contrast to ALS-iPSC CM, CsA failed to prevent the death of ALS SOD1 mutant MNs (FIG. 18, panels e-g). CsA is also an immunosuppressant. The fact that ALS iPSC-CM stabilizes mitochondria and the hyperinflammatory response in ALS MN without immunosuppression potentially makes it a better therapeutic.

Our time-course study suggests that an early diagnostic and immediate treatment are needed for the optimal efficacy of PSC-CM and the autologous ALS iPSC-CM for meaningful attenuation of the pathologies, which is the case with therapies for most neurological diseases.

### CONCLUSION

Neuronal cell death is causal in many neurodegenerative diseases, including age-related loss of memory and dementias (such as Alzheimer's Disease), Parkinson's Disease, strokes, as well as diseases that afflict broad ages, i.e., traumatic brain injury, spinal cord injury, ALS, and spinal muscle atrophy. These diseases are characterized by neuroinflammation and oxidative cell damage, many involve perturbed proteostasis and all are devastating and without a cure. Our work describes a feasible meaningful disease-minimizing treatment for ALS and suggests a clinical capacity for treating a broad class of diseases of neurodegeneration, and excessive cell apoptosis [14, 113-116].

### REFERENCES FOR EXAMPLE 2

1. Check W A. Amyotrophic lateral sclerosis. Longo D L, editor. J Am Med Assoc. Massachusetts Medical Society; 1979; 242:319.
2. Aoyama K, Nakaki T. Impaired glutathione synthesis in neurodegeneration. Int J Mol Sci. Multidisciplinary Digital Publishing Institute; 2013; 14:21021-44.
3. Gurney M E, Pu H, Chiu A Y, Dal Canto M C, Polchow C Y, Alexander D D, et al. Motor neuron degeneration in mice that express a human Cu,Zn superoxide dismutase mutation. Science (80-). 1994; 264:1772-5.
4. Rakhit R, Cunningham P, Furtos-Matei A, Dahan S, Qi X F, Crow J P, et al. Oxidation-induced misfolding and aggregation of superoxide dismutase and its implications for amyotrophic lateral sclerosis. J Biol Chem. American Society for Biochemistry and Molecular Biology; 2002; 277:47551-6.
5. Ezzi S A, Urushitani M, Julien J P. Wild-type superoxide dismutase acquires binding and toxic properties of ALS-linked mutant forms through oxidation. J Neurochem. John Wiley & Sons, Ltd; 2007; 102:170-8.
6. Barber S C, Mead R J, Shaw P J. Oxidative stress in ALS: A mechanism of neurodegeneration and a therapeutic target. Biochim. Biophys. Acta—Mol. Basis Dis. Elsevier; 2006. p. 1051-67.
7. Rotunno M S, Bosco D A. An emerging role for misfolded wild-type SOD1 in sporadic ALS pathogenesis. Front. Cell. Neurosci. Frontiers; 2013. p. 253.
8. Johnston J A, Dalton M J, Gurney M E, Kopito R R. Formation of high molecular weight complexes of mutant Cu,Zn-superoxide dismutase in a mouse model for familial amyotrophic lateral sclerosis. Proc Natl Acad Sci USA. National Academy of Sciences; 2000; 97:12571-6.
9. Pun S, Santos A F, Saxena S, Xu L, Caroni P. Selective vulnerability and pruning of phasic motoneuron axons in motoneuron disease alleviated by CNTF. Nat Neurosci. Nature Publishing Group; 2006; 9:408-19.
10. Yamashita T, Kushida Y, Wakao S, Tadokoro K, Nomura E, Omote Y, et al. Therapeutic benefit of Muse cells in a mouse model of amyotrophic lateral sclerosis. Sci Rep. Nature Research; 2020; 10:1-11.
11. Patel P, Kriz J, Gravel M, Soucy G, Bareil C, Gravel C, et al. Adeno-associated virus-mediated delivery of a recombinant single-chain antibody against misfolded superoxide dismutase for treatment of amyotrophic lateral sclerosis. Mol Ther. Nature Publishing Group; 2014; 22:498-510.
12. Zakrzewski W, Dobrzynski M, Szymonowicz M, Rybak Z. Stem cells: Past, present, and future [Internet]. Stem Cell Res. Ther. BioMed Central Ltd.; 2019. p. 68.
13. Comley L, Allodi I, Nichterwitz S, Nizzardo M, Simone C, Corti S, et al. Motor neurons with differential vulnerability to degeneration show distinct protein signatures in health and ALS. Neuroscience. Elsevier Ltd; 2015; 291:216-29.
14. Fujimori K, Ishikawa M, Otomo A, Atsuta N, Nakamura R, Akiyama T, et al. Modeling sporadic ALS in iPSC-derived motor neurons identifies a potential therapeutic agent. Nat Med. Nature Publishing Group; 2018; 24:1579-89.
15. Chen H, Qian K, Du Z, Cao J, Petersen A, Liu H, et al. Modeling ALS with iPSCs reveals that mutant SOD1 misregulates neurofilament balance in motor neurons. Cell Stem Cell. Cell Press; 2014; 14:796-809.
16. Li M, Ona V O, Gudgan C, Chen M, Jackson-Lewis V, Andrews U, et al. Functional role of caspase-1 and caspase-3 in an ALS transgenic mouse model. Science (80-). American Association for the Advancement of Science; 2000; 288:335-9.
17. Kostic V, Jackson-Lewis V, De Bilbao F, Dubois-Dauphin M, Przedborski S. Bcl-2: Prolonging life in a transgenic mouse model of familial amyotrophic lateral sclerosis. Science (80-). American Association for the Advancement of Science; 1997; 277:559-62.
18. Gutierrez-Aranda I, Ramos-Mejia V, Bueno C, Munoz-Lopez M, Real P J, Mácia A, et al. Human induced pluripotent stem cells develop teratoma more efficiently and faster than human embryonic stem cells regardless the site of injection. Stem Cells. Wiley-Blackwell; 2010. p. 1568-70.
19. Yousef H, Conboy M J, Mamiya H, Zeiderman M, Schlesinger C, Schaffer D V., et al. Mechanisms of action of hESC-secreted proteins that enhance human and mouse myogenesis. Aging (Albany NY). Impact Journals LLC; 2014; 6:602-20.
20. Yousef H, Conboy M J, Li J, Zeiderman M, Vazin T, Schlesinger C, et al. hESC-secreted proteins can be enriched for multiple regenerative therapies by heparin-binding. Aging (Albany NY). Impact Journals LLC; 2013; 5:357-72.
21. Fukuoka H, Suga H, Narita K, Watanabe R, Shintani S. The Latest Advance in Hair Regeneration Therapy Using Proteins Secreted by Adipose-Derived Stem Cells. Am J Cosmet Surg. SAGE Publications; 2012; 29:273-82.
22. Sagaradze G, Grigorieva 0, Nimiritsky P, Basalova N, Kalinina N, Akopyan Z, et al. Conditioned medium from human mesenchymal stromal cells: Towards the clinical translation. Int J Mol Sci. MDPI AG; 2019; 20.
23. Yang D, Wang W, Li L, Peng Y, Chen P, Huang H, et al. The Relative Contribution of Paracine Effect versus Direct Differentiation on Adipose-Derived Stem Cell Transplantation Mediated Cardiac Repair. PLoS One. 2013; 8.
24. Takahashi M, Li T S, Suzuki R, Kobayashi T, Ito H, Ikeda Y, et al. Cytokines produced by bone marrow cells can contribute to functional improvement of the infarcted heart by protecting cardiomyocytes from ischemic injury. Am J Physiol—Hear Circ Physiol. 2006; 291:886-93.
25. Qu Q, Li D, Louis K R, Li X, Yang H, Sun Q, et al. High-efficiency motor neuron differentiation from human pluripotent stem cells and the function of Islet-1. Nat Commun. Nature Publishing Group; 2014; 5.
26. Wang Y, Zhang L, Li Y, Chen L, Wang X, Guo W, et al. Exosomes/microvesicles from induced pluripotent stem cells deliver cardioprotective miRNAs and prevent cardiomyocyte apoptosis in the ischemic myocardium. Int J Cardiol. Elsevier Ireland Ltd; 2015; 192:61-9.
27. Metsalu T, Vilo J. ClustVis: A web tool for visualizing clustering of multivariate data using Principal Component Analysis and heatmap. Nucleic Acids Res. Oxford Academic; 2015; 43:W566-70.
28. Fontanilla C V., Gu H, Liu Q, Zhu T Z, Zhou C, Johnstone B H, et al. Adipose-derived Stem Cell Conditioned Media Extends Survival time of a mouse model of Amyotrophic Lateral Sclerosis. Sci Rep. Nature Publishing Group; 2015; 5:1-11.
29. Hatzipetros T, Kidd J D, Moreno A J, Thompson K, Gill A, Vieira F G. A quick phenotypic neurological scoring system for evaluating disease progression in the SOD1-G93A mouse model of ALS. J Vis Exp. Journal of Visualized Experiments; 2015; 2015:53257.
30. Elabd C, Cousin W, Upadhyayula P, Chen R Y, Chooljian M S, Li J, et al. Oxytocin is an age-specific circulating hormone that is necessary for muscle maintenance and regeneration. Nat Commun. Nature Publishing Group; 2014; 5:1-11.
31. Cai M D, Choi S M, Yang E J. The effects of bee venom acupuncture on the central nervous system and muscle in an animal hsOD1G93A mutant. Toxins (Basel). Multidisciplinary Digital Publishing Institute; 2015; 7:846-58.
32. Wang J, Zuzzio K, Walker C L. Systemic dental pulp stem cell secretome therapy in a mouse model of amyotrophic lateral sclerosis. Brain Sci. MDPI AG; 2019; 9:165.
33. Bigarella C L, Liang R, Ghaffari S. Stem cells and the impact of ROS signaling. Dev. 2014; 141:4206-18.
34. Tarng D C, Tseng W C, Lee P Y, Chiou S H, Hsieh S L. Induced pluripotent stem cell-derived conditioned medium attenuates acute kidney injury by downregulating the oxidative stress-related pathway in ischemia-reperfusion rats. Cell Transplant. Cognizant Communication Corporation; 2016; 25:517-30.
35. Alves C J, De Santana L P, Dos Santos A J D, De Oliveira G P, Duobles T, Scorisa J M, et al. Early motor and electrophysiological changes in transgenic mouse model of amyotrophic lateral sclerosis and gender differences on clinical outcome. Brain Res. Elsevier; 2011; 1394:90-104.
36. Rabinovich-Toidman P, Rabinovich-Nikitin I, Ezra A, Barbiro B, Fogel H, Slutsky I, et al. Mutant SOD1 increases APP expression and phosphorylation in cellular and animal models of ALS. Lakshmana M K, editor. PLoS One. Public Library of Science; 2015; 10:e0143420.
37. Haenggeli C, Julien J P, Lee Mosley R, Perez N, Dhar A, Gendelman H E, et al. Therapeutic immunization with a glatiramer acetate derivative does not alter survival in G93A and G37R SOD1 mouse models of familial ALS. Neurobiol Dis. Academic Press; 2007; 26:146-52.
38. Ari C, Poff A M, Held H E, Landon C S, Goldhagen C R, Mavromates N, et al. Metabolic therapy with deanna protocol supplementation delays disease progression and extends survival in amyotrophic lateral sclerosis (ALS) mouse model. Borchelt D R, editor. PLoS One. Public Library of Science; 2014; 9:e103526.
39. Cappello V, Francolini M. Neuromuscular junction dismantling in amyotrophic lateral sclerosis [Internet]. Int. J. Mol. Sci. MDPI AG; 2017.
40. Clark J A, Southam K A, Blizzard C A, King A E, Dickson T C. Axonal degeneration, distal collateral branching and neuromuscular junction architecture alterations occur prior to symptom onset in the SOD1G93A mouse model of amyotrophic lateral sclerosis. J Chem Neuroanat. Elsevier B.V.; 2016; 76:35-47.
41. Bruneteau G, Bauché S, Gonzalez de Aguilar J L, Brochier G, Mandjee N, Tanguy M-L, et al. Endplate denervation correlates with Nogo-A muscle expression in amyotrophic lateral sclerosis patients. Ann Clin Transl Neurol. Wiley; 2015; 2:362-72.
42. Basu J, Ludlow J W. Exosomes for repair, regeneration and rejuvenation. Expert Opin Biol Ther. Taylor & Francis; 2016; 16:489-506.
43. Duffy L M, Chapman A L, Shaw P J, Grierson A J. Review: The role of mitochondria in the pathogenesis of amyotrophic lateral sclerosis [Internet]. Neuropathol. Appl. Neurobiol. John Wiley & Sons, Ltd; 2011. p. 336-52.
44. Smith E F, Shaw P J, De Vos K J. The role of mitochondria in amyotrophic lateral sclerosis. Neurosci Lett. Elsevier; 2019; 710:132933.
45. Sultani M, Stringer A M, Bowen J M, Gibson R J. Anti-Inflammatory Cytokines: Important Immunoregulatory Factors Contributing to Chemotherapy-Induced Gastrointestinal Mucositis. Chemother Res Pract. Hindawi Limited; 2012; 2012:1-11.
46. Liu B, Hong J S. Role of microglia in inflammation-mediated neurodegenerative diseases: Mechanisms and strategies for therapeutic intervention. J Pharmacol Exp Ther. J Pharmacol Exp Ther, 2003; 304:1-7.
47. Yu C H, Davidson S, Harapas C R, Hilton J B, Mlodzianoski M J, Laohamonthonkul P, et al. TDP-43 Triggers Mitochondrial DNA Release via mPTP to Activate cGAS/STING in ALS. Cell. Cell Press; 2020; 183:636-649.e18.
48. Sakamoto K, Karelina K, Obrietan K. CREB: A multifaceted regulator of neuronal plasticity and protection. J Neurochem. NIH Public Access; 2011; 116:1-9.
49. Huang J Y, Hong Y T, Chuang J I. Fibroblast growth factor 9 prevents MPP+-induced death of dopaminergic neurons and is involved in melatonin neuroprotection in vivo and in vitro. J Neurochem. John Wiley & Sons, Ltd; 2009; 109:1400-12.
50. Valable S, Bellail A, Lesné S, Liot G, MacKenzie E T, Vivien D, et al. Angiopoietin-1-induced phosphatidyl-inositol 3-kinase activation prevents neuronal apoptosis. FASEB J. Wiley; 2003; 17:1-19.
51. Subramanian V, Crabtree B, Acharya K R. Human angiogenin is a neuroprotective factor and amyotrophic lateral sclerosis associated angiogenin variants affect neurite extension/pathfinding and survival of motor neurons. Hum Mol Genet. 2008; 17:130-49.
52. Zhou S, Wu H, Zeng C, Xiong X, Tang S, Tang Z, et al. Apolipoprotein e protects astrocytes from hypoxia and glutamate-induced apoptosis. FEBS Lett. No longer published by Elsevier; 2013; 587:254-8.
53. Tejima E, Guo S, Murata Y, Arai K, Lok J, Van Leyen K, et al. Neuroprotective effects of overexpressing tissue inhibitor of metalloproteinase TIMP-1. J Neurotrauma. 2009; 26:1935-41.
54. Ashutosh, Chao C, Borgmann K, Brew K, Ghorpade A. Tissue inhibitor of metalloproteinases-1 protects human neurons from staurosporine and HIV-1-induced apoptosis: Mechanisms and relevance to HIV-1-associated dementia. Cell Death Dis. Nature Publishing Group; 2012; 3:1-9.
55. Huynh H D, Zheng J, Umikawa M, Zhang C, Silvany R, lizuka S, et al. IGF binding protein 2 supports the survival and cycling of hematopoietic stem cells. Blood. 2011; 118:3236-43.
56. Migita T, Narita T, Asaka R, Miyagi E, Nagano H, Nomura K, et al. Role of insulin-like growth factor binding protein 2 in lung adenocarcinoma: IGF-independent antiapoptotic effect via caspase-3. Am J Pathol. 2010; 176:1756-66.
57. Fletcher L, Isgor E, Sprague S, Williams L H, Alajajian B B, Jimenez D F, et al. Spatial distribution of insulin-like growth factor binding protein-2 following hypoxic-ischemic injury. BMC Neurosci. 2013; 14.
58. Stein T D, Anders N J, DeCarli C, Chan S L, Mattson M P, Johnson J A. Neutralization of transthyretin reverses the neuroprotective effects of secreted amyloid precursor protein (APP) in APPSw mice resulting in tau phosphorylation and loss of hippocampal neurons: Support for the amyloid hypothesis. J Neurosci. 2004; 24:7707-17.
59. Akbar M T, Lundberg A M C, Liu K, Vidyadaran S, Wells K E, Dolatshad H, et al. The neuroprotective effects of heat shock protein 27 overexpression in transgenic animals against kainate-induced seizures and hippocampal cell death. J Biol Chem. J Biol Chem; 2003; 278:19956-65.
60. Toth M E, Gonda S, Vigh L, Santha M. Neuroprotective effect of small heat shock protein, Hsp27, after acute and chronic alcohol administration. Cell Stress Chaperones. Springer; 2010; 15:807-17.
61. Venugopal A, Sundaramoorthy K, Vellingiri B. Therapeutic potential of Hsp27 in neurological diseases. Egypt J Med Hum Genet. SpringerOpen; 2019; 20:1-8.
62. Dias V, Junn E, Mouradian M M. The Role of Oxidative Stress in Parkinson's Disease. J Parkinsons Dis. NIH Public Access; 2013; 3:461.
63. Huang W J, Zhang X, Chen W W. Role of oxidative stress in Alzheimer's disease. Biomed Reports. Spandidos Publications; 2016; 4:519.
64. Jang S E, Qiu L, Chan L L, Tan E K, Zeng L. Current Status of Stem Cell-Derived Therapies for Parkinson's Disease: From Cell Assessment and Imaging Modalities to Clinical Trials [Internet]. Front. Neurosci. Frontiers Media S.A.; 2020. p. 1077.
65. Song C G, Zhang Y Z, Wu H N, Cao X L, Guo C J, Li Y Q, et al. Stem cells: A promising candidate to treat neurological disorders. Neural Regen Res. Wolters Kluwer Medknow Publications; 2018; 13:1294-304.
66. Gorecka J, Kostiuk V, Fereydooni A, Gonzalez L, Luo J, Dash B, et al. The potential and limitations of induced pluripotent stem cells to achieve wound healing [Internet]. Stem Cell Res. Ther. BioMed Central Ltd.; 2019. p. 87.
67. Soldner F, Laganière J, Cheng A W, Hockemeyer D, Gao Q, Alagappan R, et al. Generation of isogenic pluripotent stem cells differing exclusively at two early onset parkinson point mutations. Cell. Cell Press; 2011; 146:318-31.
68. Tucker B A, Mullins R F, Streb L M, Anfnson K, Eyestone M E, Kaalberg E, et al. Patient-specifc iPSC-derived photoreceptor precursor cells as a means to investigate retinitis pigmentosa. Elife. eLife Sciences Publications Ltd; 2013; 2013.
69. Douvaras P, Wang J, Zimmer M, Hanchuk S, O'Bara M A, Sadiq S, et al. Efficient generation of myelinating oligodendrocytes from primary progressive multiple sclerosis patients by induced pluripotent stem cells. Stem Cell Reports. Cell Press; 2014; 3:250-9.
70. Gonzalez-Begne M, Lu B, Han X, Hagen F K, Hand A R, Melvin J E, et al. Proteomic analysis of human parotid gland exosomes by multidimensional protein identification technology (MudPIT). J Proteome Res. American Chemical Society; 2009; 8:1304-14.
71. Kim H S, Choi D Y, Yun S J, Choi S M, Kang J W, Jung J W, et al. Proteomic analysis of microvesicles derived from human mesenchymal stem cells. J Proteome Res. American Chemical Society; 2012; 11:839-49.
72. Pires A O, Mendes-Pinheiro B, Teixeira F G, Anjo S I, Ribeiro-Samy S, Gomes E D, et al. Unveiling the Differences of Secretome of Human Bone Marrow Mesenchymal Stem Cells, Adipose Tissue-Derived Stem Cells, and Human Umbilical Cord Perivascular Cells: A Proteomic Analysis. Stem Cells Dev. Mary Ann Liebert Inc.; 2016; 25:1073-83.
73. Verghese P B, Castellano J M, Garai K, Wang Y, Jiang H, Shah A, et al. ApoE influences amyloid-β (Aβ) clearance despite minimal apoE/Aβ association in physiological conditions. Proc Natl Acad Sci USA. National Academy of Sciences; 2013; 110:E1807-16.
74. Sebastià J, Kieran D, Breen B, King M A, Netteland D F, Joyce D, et al. Angiogenin protects motoneurons against hypoxic injury. Cell Death Differ. Nature Publishing Group; 2009; 16:1238-47.
75. Kieran D, Sebastia J, Greenway M J, King M A, Connaughton D, Concannon C G, et al. Control of motoneuron survival by angiogenin. J Neurosci. Society for Neuroscience; 2008; 28:14056-61.
76. Valable S, Bellail A, Lesné S, Liot G, MacKenzie E T, Vivien D, et al. Angiopoietin-1-induced phosphatidyl-inositol 3-kinase activation prevents neuronal apoptosis. FASEB J. John Wiley & Sons, Ltd; 2003; 17:1-19.
77. Jiang H, Zhang F, Yang J, Han S. Angiopoietin-1 ameliorates inflammation-induced vascular leakage and improves functional impairment in a rat model of acute experimental autoimmune encephalomyelitis. Exp Neurol. Exp Neurol; 2014; 261:245-57.
78. Kosacka J, Figiel M, Engele J, Hilbig H, Majewski M, Spanel-Borowski K. Angiopoietin-1 promotes neurite outgrowth from dorsal root ganglion cells positive for Tie-2 receptor. Cell Tissue Res. Springer, 2005; 320:11-9.
79. Yin J, Gong G, Liu X. Angiopoietin: A Novel Neuroprotective/Neurotrophic Agent. Neuroscience. IBRO; 2019; 411:177-84.
80. Patel Y J K, Payne Smith M D, De Belleroche J, Latchman D S. Hsp27 and Hsp70 administered in combination have a potent protective effect against FALS-associated SOD1-mutant-induced cell death in mammalian neuronal cells. Mol Brain Res. Elsevier, 2005; 134:256-74.
81. Chen Y T, Tsai M J, Hsieh N, Lo M J, Lee M J, Cheng H, et al. The superiority of conditioned medium derived from rapidly expanded mesenchymal stem cells for neural repair. Stem Cell Res Ther. BioMed Central Ltd.; 2019; 10:1-15.
82. Hilkens P, Fanton Y, Martens W, Gervois P, Struys T, Politis C, et al. Pro-angiogenic impact of dental stem cells in vitro and in vivo. Stem Cell Res. Elsevier, 2014; 12:778-90.
83. Aparicio-Erriu I M, Prehn J H M. Molecular mechanisms in amyotrophic lateral sclerosis: The role of angiogenin, a secreted rnase. Front Neurosci. Frontiers; 2012; 0:167.
84. Subramanian V, Feng Y. A new role for angiogenin in neurite growth and pathfinding: Implications for amyotrophic lateral sclerosis. Hum Mol Genet. Oxford Academic; 2007; 16:1445-53.
85. Xiang J, Hu J, Shen T, Liu B, Hua F, Zan K, et al. Bone marrow mesenchymal stem cells-conditioned medium enhances vascular remodeling after stroke in type 2 diabetic rats. Neurosci Lett. Elsevier, 2017; 644:62-6.
86. Zhao Q, Hu J, Xiang J, Gu Y, Jin P, Hua F, et al. Intranasal administration of human umbilical cord mesenchymal stem cells-conditioned medium enhances vascular remodeling after stroke. Brain Res. Elsevier; 2015; 1624:489-96.
87. Pizurki L, Zhou Z, Glynos K, Roussos C, Papapetropoulos A. Angiopoietin-1 inhibits endothelial permeability, neutrophil adherence and IL-8 production. Br J Pharmacol. John Wiley & Sons, Ltd; 2003; 139:329-36.
88. Bakthisaran R, Tangirala R, Rao C M. Small heat shock proteins: Role in cellular functions and pathology. Biochim Biophys Acta—Proteins Proteomics. Elsevier, 2015; 1854:291-319.
89. Weeks S D, Muranova L K, Heirbaut M, Beelen S, Strelkov S V., Gusev N B. Characterization of human small heat shock protein HSPB1 α-crystallin domain localized mutants associated with hereditary motor neuron diseases. Sci Reports 2018 81. Nature Publishing Group; 2018; 8:1-15.
90. Yu A L, Fuchshofer R, Birke M, Kampik A, Bloemendal H, Welge-Lüssen U. Oxidative Stress and TGF-β2 Increase Heat Shock Protein 27 Expression in Human Optic Nerve Head Astrocytes. Invest Ophthalmol Vis Sci. The Association for Research in Vision and Ophthalmology; 2008; 49:5403-11.
91. Tóth ME, Szegedi V, Varga E, Juhász G, Horváth J, Borbély E, et al. Overexpression of Hsp27 ameliorates symptoms of Alzheimer's disease in APP/PS1 mice. Cell Stress Chaperones. Cell Stress and Chaperones; 2013; 18:759-71.
92. Zhou B R, Xu Y, Xu Y, Guo S L, Wang Y, Zhu F, et al. The effect of conditioned media of Adipose-derived stem cells on wound healing after ablative fractional carbon dioxide laser resurfacing. Biomed Res Int. 2013; 2013.
93. Kay A G, Long G, Tyler G, Stefan A, Broadfoot S J, Piccinini A M, et al. Mesenchymal Stem Cell-Conditioned Medium Reduces Disease Severity and Immune Responses in Inflammatory Arthritis. Sci Rep. Nature Publishing Group; 2017; 7.
94. Dahbour S, Jamali F, Alhattab D, Al-Radaideh A, Ababneh 0, Al-Ryalat N, et al. Mesenchymal stem cells and conditioned media in the treatment of multiple sclerosis patients: Clinical, ophthalmological and radiological assessments of safety and efficacy. CNS Neurosci Ther. Blackwell Publishing Ltd; 2017; 23:866-74.
95. Pawitan J A. Prospect of stem cell conditioned medium in regenerative medicine. Biomed Res Int. Hindawi Limited; 2014; 2014.
96. Boland M J, Nazor K L, Loring J F. Epigenetic regulation of pluripotency and differentiation. Circ Res. NIH Public Access; 2014; 115:311.
97. Sparmann A, Van Lohuizen M. Polycomb silencers control cell fate, development and cancer [Internet]. Nat. Rev. Cancer. Nat Rev Cancer; 2006. p. 846-56.
98. O'Carroll D, Erhardt S, Pagani M, Barton S C, Surani M A, Jenuwein T. The Polycomb-Group Gene Ezh2 Is Required for Early Mouse Development. Mol Cell Biol. American Society for Microbiology; 2001; 21:4330-6.
99. Collinson A, Collier A J, Morgan N P, Sienerth A R, Chandra T, Andrews S, et al. Deletion of the Polycomb-Group Protein EZH2 Leads to Compromised Self-Renewal and Differentiation Defects in Human Embryonic Stem Cells. Cell Rep. Elsevier Company; 2016; 17:2700-14.
100. Sher F, Rößler R, Brouwer N, Balasubramaniyan V, Boddeke E, Copray S. Differentiation of Neural Stem Cells into Oligodendrocytes: Involvement of the Polycomb Group Protein Ezh2. Stem Cells. Wiley; 2008; 26:2875-83.
101. Aoki R, Chiba T, Miyagi S, Negishi M, Konuma T, Taniguchi H, et al. The polycomb group gene product Ezh2 regulates proliferation and differentiation of murine hepatic stem/progenitor cells. J Hepatol. J Hepatol; 2010; 52:854-63.
102. Juan A H, Kumar R M, Marx J G, Young R A, Sartorelli V. Mir-214-Dependent Regulation of the Polycomb Protein Ezh2 in Skeletal Muscle and Embryonic Stem Cells. Mol Cell. Mol Cell; 2009; 36:61-74.
103. Yao Y, Hu H, Yang Y, Zhou G, Shang Z, Yang X, et al. Downregulation of enhancer of zeste homolog 2 (EZH2) is essential for the induction of autophagy and apoptosis in colorectal cancer cells. Genes (Basel). 2016; 7:83.
104. Qi W, Chan H M, Teng L, Li L, Chuai S, Zhang R, et al. Selective inhibition of Ezh2 by a small molecule inhibitor blocks tumor cells proliferation. Proc Natl Acad Sci USA. National Academy of Sciences; 2012; 109:21360-5.
105. Lee Y C, Chang W W, Chen Y Y, Tsai Y H, Chou Y H, Tseng H C, et al. Hsp90a mediates BMI1 expression in breast cancer stem/progenitor cells through facilitating nuclear translocation of c-Myc and EZH2. Int J Mol Sci. Multidisciplinary Digital Publishing Institute; 2017; 18:1986.
106. Huang Q, He S, Tian Y, Gu Y, Chen P, Li C, et al. Hsp90 inhibition destabilizes Ezh2 protein in alloreactive T cells and reduces graft-versus-host disease in mice. Blood. American Society of Hematology; 2017; 129:2737-48.
107. Moore H M, Gonzalez M E, Toy K A, Cimino-Mathews A, Argani P, Kleer C G. EZH2 inhibition decreases p38 signaling and suppresses breast cancer motility and metastasis. Breast Cancer Res Treat. NIH Public Access; 2013; 138:741-52.
108. Herrera-Merchan A, Arranz L, Ligos J M, De Molina A, Dominguez O, Gonzalez S. Ectopic expression of the histone methyltransferase Ezh2 in haematopoietic stem cells causes myeloproliferative disease. Nat Commun. Nature Publishing Group; 2012; 3:1-12.
109. Guo Y L, Chakraborty S, Rajan S S, Wang R, Huang F. Effects of oxidative stress on mouse embryonic stem cell proliferation, apoptosis, senescence, and self-renewal. Stem Cells Dev. Mary Ann Liebert, Inc. 140 Huguenot Street, 3rd Floor New Rochelle, NY 10801 USA; 2010; 19:1321-31.
110. Zhang J, Khvorostov I, Hong J S, Oktay Y, Vergnes L, Nuebel E, et al. UCP2 regulates energy metabolism and differentiation potential of human pluripotent stem cells. EMBO J. Wiley-VCH Verlag; 2011; 30:4860-73.
111. Mandal S, Lindgren A G, Srivastava A S, Clark A T, Banerjee U. Mitochondrial function controls proliferation and early differentiation potential of embryonic stem cells. Stem Cells. John Wiley & Sons, Ltd; 2011; 29:486-95.
112. Zhou W, Choi M, Margineantu D, Margaretha L, Hesson J, Cavanaugh C, et al. HIF1α induced switch from bivalent to exclusively glycolytic metabolism during ESC-to-EpiSC/hESC transition. EMBO J. John Wiley & Sons, Ltd; 2012; 31:2103-16.
113. Inoue H, Tsukita K, Iwasato T, Suzuki Y, Tomioka M, Tateno M, et al. The crucial role of caspase-9 in the disease progression of a transgenic ALS mouse model. EMBO J. 2003; 22:6665-74.
114. Pedersen W A, Luo H, Kruman I, Kasarskis E, Mattson M P. The prostate apoptosis response-4 protein participates in motor neuron degeneration in amyotrophic lateral sclerosis. FASEB J. 2000; 14:913-24.
115. Guerrero E N, Mitra J, Wang H, Rangaswamy S, Hegde P M, Basu P, et al. Amyotrophic lateral sclerosis-associated TDP-43 mutation Q331K prevents nuclear translocation of XRCC4-DNA ligase 4 complex and is linked to genome damage-mediated neuronal apoptosis. Hum Mol Genet. 2019; 28:2459-76.
116. Sathasivam S, Shaw P J. Apoptosis in amyotrophic lateral sclerosis—What is the evidence? Lancet Neurol. Elsevier, 2005; 4:500-9.

### List of Abbreviations

ALS Amyotrophic Lateral Sclerosis
PSCs Pluripotent Stem Cell
MN Motor Neurons
SOD1 Superoxide Dismutase 1
ESCs Embryonic Stem Cells
iPSCs Induced Pluripotent Stem Cells
CM Conditioned Medium
CsA Cyclosporin A
EB Embryoid Body
MNP Motor Neuron Precursor
HBP Heparin-Binding Proteins
PBS Phosphate-Buffered Saline
RA Retinoic Acid
VPA Valproic Acid
NBM Neural Basal Medium
PCR Polymerase Chain Reaction
NMJ Neuromuscular Junction
dF-CM Conditioned Medium From Differentiated Fibroblast Cells
CC3 Cleaved Caspase3
AchR Acetylcholine Receptor
GA Gastrocnemius
TA Tibialis Anterior
NaA Nanomycin A
LSD1 Lsd1 Inhibitor
5Aza 5 Azacytidine
MFI Mean Fluorescence Intensity

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of protecting mammalian cells from oxidative stress, and/or mitochondrial dysfunction, and/or inflammatory gene expression, and/or protein aggregation, and/or toxin induced cell death, said method comprising:
contacting said cells with an effective amount of one or more of the following:
a) unfractionated iPS cell derived secretome, where said iPS cells are derived from a mammal with a neurodegenerative pathology or a healthy mammal;
b) heparin bound and/or unbound fractions of iPS cell derived secretome, where said iPS cells are derived from a mammal with a neurodegenerative pathology or a healthy mammal;
c) a combination of soluble and exosome fractions of iPS cell derived secretome, where said iPS cells are derived from a mammal with a neurodegenerative pathology or a healthy mammal;
d) proteins that are secreted by induced pluripotent stem cells (iPSCs) derived from a mammal with a neurodegenerative pathology or a healthy mammal where said proteins are secreted when said iPSC cells are pluripotent or biologically active fragments of said proteins and/or biologically active analogs of said proteins; and/or
e) combinations of 2 or more proteins shown in Table 9, or biologically active fragments of said proteins and/or biologically active analogs of said proteins.

2. The method of claim 1, wherein said method comprises a method of protecting neuron from oxidative stress, and/or protein aggregation, and/or toxin-induced cell death.

3. The method of claim 1, wherein said method comprises contacting said cells with an effective amount of unfractionated iPS cell derived secretome, where said iPS cells are derived from a mammal with a neurodegenerative pathology and/or from a healthy mammal.

4. The method of claim 1, wherein said method comprises contacting said cells with an effective amount of heparin bound fraction of iPS cell derived secretome, where said iPS cells are derived from a mammal with a neurodegenerative pathology and/or a from a healthy mammal.

5. The method of claim 1, wherein said method comprises contacting said neurons with an effective amount of heparin unbound fraction of iPS cell derived secretome, where said iPS cells are derived from a mammal with a neurodegenerative pathology and/or from a healthy mammal.

6. The method of claim 1, wherein said method comprises contacting said neurons with an effective amount of a plurality of proteins that are secreted by induced pluripotent stem cells (iPSCs) derived from a mammal with a neurodegenerative pathology and/or from a healthy mammal where said proteins are secreted when said iPSC cells are pluripotent or biologically active fragments of said proteins.

7. The method according to any one of claims 1-6, wherein said cells comprise neurons.

8. The method according to any one of claims 1-7, wherein said iPSCs are derived from a mammal with a neurodegenerative pathology.

9. The method according to any one of claims 1-7, wherein said iPSCs are derived from a healthy mammal.

10. The method according to any one of claims 1-9, wherein:
said iPS cell derived secretome; and/or
said heparin bound fraction of iPS cell derived secretome; and/or
said heparin unbound fraction of iPS cell derived secretome; and/or
said proteins secreted by induced pluripotent stem cells (iPS cells);
are from iPS cells derived from a healthy mammal and/or from a mammal having a neuropathology selected from the group consisting of a motor neuron disease, Alzheimer's disease, Parkinson's disease, Huntington's disease, HIV-1 associated neurological degeneration, neurodegeneration associated with an ischemic event, neurodegeneration associated with traumatic brain injury (TBI), neurodegeneration associated with a spinal cord injury, drug-environmental toxin-induced neurodegeneration, and glaucoma.

11. The method of claim 10, wherein said neuropathology comprises a pathology selected from the group consisting of a motor neuron disease, Alzheimer's disease, Parkinson's disease, Huntington's disease, HIV-1 associated neurological degeneration, neurodegeneration associated with an ischemic event, neurodegeneration associated with traumatic brain injury (TBI), neurodegeneration associated with a spinal cord injury, drug-environmental toxin-induced neurodegeneration, and glaucoma.

12. The method of claim 11, wherein said neuropathology comprises a motor neuron disease selected from the group consisting of Amyotrophic lateral sclerosis (ALS), spinal muscular atrophy (SMA), progressive bulbar palsy (PBP), primary lateral sclerosis (PLS), pseudobulbar palsy, and progressive muscular atrophy (PMA).

13. The method of claim 12, wherein said motor neuron disease comprises ALS.

14. The method of claim 12, wherein said motor neuron disease comprises SMA.

15. The method according to any one of claims 1-14, wherein said proteins comprise or consist of proteins whose production and/or secretion by IPS cells is reduced or absent when said iPSCs differentiate into the embryoid bodies or their derived lineages, such as fibroblasts.

16. The method according to any one of claims 1-15, wherein said proteins comprise or consist of proteins whose neuroprotective activities are reduced or absent when said iPSCs differentiate into the embryoid bodies or their derived lineages, such as fibroblasts.

17. The method according to any one of claims 1-16, wherein said plurality of proteins comprises a protein selected from the group consisting of angiogenin (ANG), Angiopoietin-1(ANG-1), angiopoietin like 3, apolipoprotein E (ApoE), secreted frizzled related protein 1, serpin family A member 5, thrombospondin 4, vascular endothelial growth factor B, fibroblast growth factor 11 (FGF11), and fibroblast growth factor 19 (FGF19), one or more proteins of Table 9, or biologically active fragments or analogs thereof.

18. The method according to any one of claims 1-17, wherein said plurality of proteins comprises a protein selected from the group consisting of ANG-1, ANG, IGFBP-2, TIMP-1, SERPINA5, FGF19, and ApoE, or biologically active fragments or analogs thereof.

19. The method according to any one of claims 1-18, wherein said plurality of proteins comprises two or more proteins independently selected from the group consisting of Aldolase C, Alpha Lactalbumin, ANGPTL3, ApoA2, ApoE, BCAM, MUC1, MUC16, Caspase-3, Cathepsin B, CBP, TfR, CEA, Ceruloplasmin, Chemerin, CHI3L1, CK-MB, Clusterin, C2, C5a, Corticosteroid-binding globulin, C-Peptide, Troponin T, Cytokeratin 19, BNP, ACTH, Exostosin-like 2, Ferritin, Fibrinopeptide A, FSH, GLP-1, GMNN, Hemopexin, HSP27, HSP90, IL-34, Kallikrein 2, Kallikrein 10, Lyn, NPTXR, P-Cadherin, PIM2, PPARg2, PR Isoform B, PSA-free, PTPRD, Ret, Serpin A5, SHBG, SOX2, Angiogenin, Angiopoietin-1, CCR5, CD40 Ligand, Chordin-Like-1, Cripto-1, CXCR6, EG-VEGF, ErbB3, FGF-11, FGF-19, GDF-9, GDF-11, Granzyme A, GRO, HCR, NRG1 Isoform GGF2, IFN-alpha/beta R2, IGFBP-2, IL-38, IL-7, IL-17 RB, IL-17 RD, IL-21, IL-23 R, IL-26, IL-29, Insulysin, Kremen-2, MFRP, MIF, MIP 2, MMP-9, MMP-16, MMP-25, NRG1 Isoform GGF2, Orexin-A, Oncostatin M, OX40 Ligand, PDGF-AA, PDGF-C, ROBO4, sFRP-1, SIGIRR, SMAD1, SMAD4, SMAD5, SMAD7, Spinesin, TACI, Thrombospondin-4, TIMP-1, TLR1, TRADD, TWEAK, and VEGF-B, or biologically active fragments or analogs thereof.

20. The method according to any one of claims 17-19, wherein said protein(s) comprise full-length proteins.

21. The method according to any one of claims 17-19, wherein said protein(s) comprise biologically active fragments of said proteins.

22. The method according to any one of claims 17-19, wherein said protein(s) comprise analogs of said proteins.

23. The method according to any one of claims 1-22, wherein said composition is effective to reduce cell apoptosis as compared to an untreated control.

24. The method of claim 23, wherein said composition is effective to reduce neuronal apoptosis as compared to an untreated control.

25. The method according to any one of claims 1-22, wherein said composition is effective to diminish aggregation of TDP43 as compared to an untreated control.

26. The method according to any one of claims 1-22, wherein said composition is effective to diminish neurite shrinkage as compared to an untreated control.

27. The method according to any one of claims 1-22, wherein said composition is effective to diminish mitochondrial dysfunction as compared to an untreated control.

28. The method according to any one of claims 1-22, wherein said composition is effective to diminish inflammatory gene expression as compared to an untreated control.

29. The method according to any one of claims 1-28, wherein said contacting comprises administering said plurality of proteins and/or biologically active fragments of said proteins and/or analogs thereof to a mammal in need thereof.

30. The method of claim 29, wherein said mammal in need thereof comprises a mammal diagnosed as having or as at risk for a neurodegenerative pathology or mitochondrial disease.

31. The method of claim 30, wherein said mammal in need thereof comprises a mammal having or at risk for a neurodegenerative pathology selected from the group consisting of a motor neuron disease, Alzheimer's disease, Parkinson's disease, Huntington's disease, HIV-1 associated neurological degeneration, neurodegeneration associated with an ischemic event, neurodegeneration associated with traumatic brain injury (TBI), neurodegeneration associated with a spinal cord injury, drug-environmental toxin-induced neurodegeneration, and glaucoma.

32. The method of claim 31, wherein said neurodegenerative pathology comprises a motor neuron disease selected from the group consisting of wherein said motor neuron disease comprises a disease selected from the group consisting of Amyotrophic lateral sclerosis (ALS), spinal muscular atrophy (SMA type I, and/or type II, and/or type III, and/or congenital SMA), progressive bulbar palsy (PBP), pseudobulbar palsy, primary lateral sclerosis (PLS), progressive muscular atrophy (PMA), and Fazio-Londe disease.

33. The method of claim 32, wherein:
said motor neuron disease comprises amyotrophic lateral sclerosis (ALS); or
said motor neuron disease comprises SMA; or
said neurodegenerative pathology comprises methamphetamine-associated neurodegeneration; or
said neurodegenerative pathology comprises toxin-induced (e.g., environmental toxin-induced) neurodegeneration; or
said neurodegenerative pathology comprises Alzheimer's disease; or
said neurodegenerative pathology comprises Parkinson's disease; or
said neurodegenerative pathology comprises neurodegeneration associated with a stroke; or
said neurodegenerative pathology comprises neurodegeneration associated with a spinal cord injury; or
said neurodegenerative pathology comprises neurodegeneration associated with a traumatic brain injury; or
said neurodegenerative pathology comprises glaucoma.

34. The method of claim 30, wherein said mammal in need thereof comprises a mammal having or at risk for a mitochondrial disease.

35. The method of claim 34, wherein said mitochondrial disease comprises a disease selected from the group consisting of Autosomal dominant optic atrophy, Alpers Disease or Syndrome, Barth Syndrome, Beta-oxidation defects, Carnitine Deficiency, Carnitine-Acyl-Carnitine Deficiency, Chronic Progressive External Ophthalmoplegia Syndrome (CPEO), Co-Enzyme Q10 Deficiency, Complex I Deficiency, Complex II Deficiency, Complex III Deficiency/COX Deficiency, Complex V Deficiency, CPT I Deficiency, CPT II Deficiency, Creatine Deficiency Syndrome, Kearns-Sayre Syndrome (KSS), Lactic Acidosis, Leukodystrophy (LBSL), LCHA Deficiency, Leber Hereditary Optic Neuropathy, Leigh Disease or syndrome, Long-Chain Acyl-CoA Dehydrongenase Deficiency (LCA Deficiency), Luft Disease, MAD/Glutaric Aciduria Type II, Medium-Chain Acyl-CoA Dehydrongenase Deficiency (MCAD), Mitochondrial Encephalomyopathy Lactic Acidosis and Stroke-like Episodes (MELAS), Mitochondrial DNA Depletion, Mitochondrial Encephalopathy, Mitochondrial Enoyl CoA Reductase Protein Associated Neurodegeneration (MEPAN), Mitochondrial Recessive Ataxia Syndrome (MIRAS), Myoclonic Epilepsy and Ragged-Red Fiber Disease (MERRF), Myoneurogastointestinal Disorder and Encephalopathy (MNGIE), Neuropathy, Ataxia, and Retinitis Pigmentosa (NARP), Neuropathy, Ataxia, and Retinitis Pigmentosa (NARP), Pearson Syndrome, Pyruvate Carboxylase Deficiency, Pyruvate Carboxylase Deficiency, Pyruvate Dehydrogenase Deficiency (PDC deficiency), Pyruvate Dehydrogenase Deviciency (PDC Deficiency), SANDO, SCHAD, Short-Chain Acyl-CoA Dehydrogenase Deficiency (SCAD), TK2/myopathic form, and Very Long-Chain Acyl-CoA Dehydrogenase Deficiency (VLCAD).

36. The method according to any one of claims 29-35, wherein said mammal is a human.

37. The method according to any one of claims 29-35, wherein said mammal is a non-human mammal.

38. The method according to any one of claims 29-37, wherein said neuroprotection comprises protection of cortical neurons.

39. The method according to any one of claims 29-38, wherein said neuroprotection comprises protection of hippocampal neurons.

40. The method according to any one of claims 29-39, wherein said neuroprotection comprises protection of dopaminergic neurons.

41. The method according to any one of claims 29-40, wherein said neuroprotection comprises protection of spinal cord neurons.

42. The method according to any one of claims 29-41, wherein said neuroprotection comprises protection of motor neurons.

43. The method according to any one of claims 29-42, wherein said contacting comprises administration to a mammal via a route selected from the group consisting of oral delivery, isophoretic delivery, transdermal delivery, parenteral delivery, aerosol administration, administration via inhalation, intravenous administration, and rectal administration.

44. The method according to any one of claims 29-42, wherein said contacting comprises administration to the brain or spinal cord of a mammal.

45. The method according to any one of claims 29-42, wherein said contacting comprises intracerebral, ventricular or intrathecal delivery to a mammal.

46. The method according to any one of claims 29-42, wherein said contacting comprises intranasal delivery to a mammal.

47. The method according to any one of claims 29-42, wherein said contacting comprises delivery via an implant in a mammal.

48. The method according to any one of claims 1-47, wherein said plurality secretome, secretome fractions, proteins and/or biologically active fragments thereof, and/or analogs thereof are effective to inhibit apoptosis, and/or to attenuate oxidative stress, and/or to repair oxidative damage, and/or prevent protein aggregation and mislocalization and/or prevent loss of axonal, etc. neuron projections, and/or prevent denervation, and/or increase innervation, muscle mass, neuro-muscular function, prevent and attenuate paralysis and morbidity, when administered to a mammal.

49. A neuroprotective composition comprising:
a plurality of proteins (unfractionated or fractionated) that are secreted by induced pluripotent stem cells (iPSCs) derived from a mammal with a neuropathology and/or mitochondrial disease and/or from a healthy mammal, where said proteins are secreted when said iPSC cells are pluripotent; and/or
biologically active fragments of said secreted proteins; and/or
biologically active analogs of said secreted proteins.

50. The neuroprotective composition of claim 49, wherein said mitochondrial disease comprises a disease selected from the group consisting of Autosomal dominant optic atrophy, Alpers Disease or Syndrome, Barth Syndrome, Beta-oxidation defects, Carnitine Deficiency, Carnitine-Acyl-Carnitine Deficiency, Chronic Progressive External Ophthalmoplegia Syndrome (CPEO), Co-Enzyme Q10 Deficiency, Complex I Deficiency, Complex II Deficiency, Complex III Deficiency/COX Deficiency, Complex V Deficiency, CPT I Deficiency, CPT II Deficiency, Creatine Deficiency Syndrome, Kearns-Sayre Syndrome (KSS), Lactic Acidosis, Leukodystrophy (LBSL), LCHA Deficiency, Leber Hereditary Optic Neuropathy, Leigh Disease or syndrome, Long-Chain Acyl-CoA Dehydrongenase Deficiency (LCA Deficiency), Luft Disease, MAD/Glutaric Aciduria Type II, Medium-Chain Acyl-CoA Dehydrongenase Deficiency (MCAD), Mitochondrial Encephalomyopathy Lactic Acidosis and Stroke-like Episodes (MELAS), Mitochondrial DNA Depletion, Mitochondrial Encephalopathy, Mitochondrial Enoyl CoA Reductase Protein Associated Neurodegeneration (MEPAN), Mitochondrial Recessive Ataxia Syndrome (MIRAS), Myoclonic Epilepsy and Ragged-Red Fiber Disease (MERRF), Myoneurogastointestinal Disorder and Encephalopathy (MNGIE), Neuropathy, Ataxia, and Retinitis Pigmentosa (NARP), Neuropathy, Ataxia, and Retinitis Pigmentosa (NARP), Pearson Syndrome, Pyruvate Carboxylase Deficiency, Pyruvate Carboxylase Deficiency, Pyruvate Dehydrogenase Deficiency (PDC deficiency), Pyruvate Dehydrogenase Deviciency (PDC Deficiency), SANDO, SCHAD, Short-Chain Acyl-CoA Dehydrogenase Deficiency (SCAD), TK2/myopathic form, and Very Long-Chain Acyl-CoA Dehydrogenase Deficiency (VLCAD).

51. The neuroprotective composition of claim 49, wherein said neuropathology is a neuropathology selected from the group consisting of a motor neuron disease, Alzheimer's disease, Parkinson's disease, Huntington's disease, HIV-1 associated neurological degeneration, neurodegeneration associated with an ischemic event, neurodegeneration associated with traumatic brain injury (TBI), neurodegeneration associated with a spinal cord injury, drug-environmental toxin-induced neurodegeneration, and glaucoma.

52. The neuroprotective composition of claim 51, wherein said neuropathology comprises a motor neuron disease.

53. The neuroprotective composition of claim 52, wherein said motor neuron disease comprises a disease selected from the group consisting of Amyotrophic lateral sclerosis (ALS), spinal muscular atrophy (SMA), progressive bulbar palsy (PBP), primary lateral sclerosis (PLS), pseudobulbar palsy, and progressive muscular atrophy (PMA).

54. The neuroprotective composition of claim 52, wherein said motor neuron disease comprises ALS.

55. The neuroprotective composition of claim 52, wherein said motor neuron disease comprises SMA.

56. The neuroprotective composition according to any one of claims 49-55, wherein said plurality of proteins comprise or consist of proteins whose activity and/or levels are reduced or absent when said iPSCs differentiate along the embryonic fibroblast lineage.

57. The neuroprotective composition according to any one of claims 49-56, wherein said plurality of proteins comprises a protein selected from the group consisting of angiogenin (ANG), Angiopoietin-1(ANG-1), angiopoietin like 3, apolipoprotein E (ApoE), secreted frizzled related protein 1, serpin family A member 5, thrombospondin 4, vascular endothelial growth factor B, fibroblast growth factor 11 (FGF11), and fibroblast growth factor 19 (FGF19), or biologically active fragments or analogs thereof.

58. The neuroprotective composition according to any one of claims 49-57, wherein said plurality of proteins comprise or consists of one or more proteins selected from the group consisting of ANG-1, ANG, IGFBP-2, TIMP-1, SERPINA5, FGF19, and ApoE, or biologically active fragments or analogs thereof.

59. The neuroprotective composition according to any one of claims 49-57, wherein said plurality of proteins comprises two or more proteins independently selected from the group consisting of Aldolase C, Alpha Lactalbumin, ANGPTL3, ApoA2, ApoE, BCAM, MUC1, MUC16, Caspase-3, Cathepsin B, CBP, TfR, CEA, Ceruloplasmin, Chemerin, CHI3L1, CK-MB, Clusterin, C2, C5a, Corticosteroid-binding globulin, C-Peptide, Troponin T, Cytokeratin 19, BNP, ACTH, Exostosin-like 2, Ferritin, Fibrinopeptide A, FSH, GLP-1, GMNN, Hemopexin, HSP27, HSP90, IL-34, Kallikrein 2, Kallikrein 10, Lyn, NPTXR, P-Cadherin, PIM2, PPARg2, PR Isoform B, PSA-free, PTPRD, Ret, Serpin A5, SHBG, SOX2, Angiogenin, Angiopoietin-1, CCR5, CD40 Ligand, Chordin-Like-1, Cripto-1, CXCR6, EG-VEGF, ErbB3, FGF-11, FGF-19, GDF-9, GDF-11, Granzyme A, GRO, HCR, NRG1 Isoform GGF2, IFN-alpha/beta R2, IGFBP-2, IL-38, IL-7, IL-17 RB, IL-17 RD, IL-21, IL-23 R, IL-26, IL-29, Insulysin, Kremen-2, MFRP, MIF, MIP 2, MMP-9, MMP-16, MMP-25, NRG1 Isoform GGF2, Orexin-A, Oncostatin M, OX40 Ligand, PDGF-AA, PDGF-C, ROBO4, sFRP-1, SIGIRR, SMAD1, SMAD4, SMAD5, SMAD7, Spinesin, TACI, Thrombospondin-4, TIMP-1, TLR1, TRADD, TWEAK, and VEGF-B, or biologically active fragments or analogs thereof.

60. The neuroprotective composition according to any one of claims 57-59, wherein said protein(s) comprise full-length proteins.

61. The neuroprotective composition according to any one of claims 57-59, wherein said protein(s) comprise biologically active fragments of said proteins.

62. The neuroprotective composition according to any one of claims 57-59, wherein said protein(s) comprise analogs of said proteins.

63. The neuroprotective composition according to any one of claims 57-62, wherein said composition is effective to reduce neuronal apoptosis as compared to an untreated control.

64. The neuroprotective composition according to any one of claims 57-63, wherein said composition is effective to diminish aggregation of TDP43 as compared to an untreated control.

65. The neuroprotective composition according to any one of claims 57-64, wherein said composition is effective to diminish neurite shrinkage as compared to an untreated control.

66. The neuroprotective composition according to any one of claims 57-65, wherein said composition comprises a pharmaceutically acceptable carrier and is a pharmaceutical formulation.

67. The neuroprotective composition of claim 66, wherein said composition comprises a substantially sterile pharmaceutical formulation.

68. The neuroprotective composition according to any one of claims 66-67, wherein pharmaceutical formulation is formulated for administration to a mammal via a route selected from the group consisting of oral delivery, isophoretic delivery, transdermal delivery, parenteral delivery, aerosol administration, administration via inhalation, intravenous administration, and rectal administration.

69. The neuroprotective composition according to any one of claims 66-67, wherein pharmaceutical formulation is formulated for administration to the brain or spinal cord of a mammal.

70. The neuroprotective composition according to any one of claims 66-67, wherein pharmaceutical formulation is formulated for intracerebral, ventricular or intrathecal delivery to a mammal.

71. The neuroprotective composition according to any one of claims 66-67, wherein pharmaceutical formulation is formulated for intranasal delivery to a mammal.

72. The neuroprotective composition according to any one of claims 66-67, wherein pharmaceutical formulation is formulated for delivery via an implant in a mammal.

73. The neuroprotective composition according to any one of claim 49-72, wherein said composition is effective to inhibit apoptosis, and/or to attenuate oxidative stress, and/or to repair oxidative damage, and/or prevent protein aggregation and mislocalization, and/or prevent loss of axonal, etc. neuron projections, and/or prevent denervation, and/or increase innervation, muscle mass, neuro-muscular function, prevent and attenuate paralysis and morbidity when administered to a mammal.
